(19) United States
(12) Patent Application Publication
(10) Pub. No.: US 2021/0358575 A1
(43) Pub. Date: Nov. 18, 2021

(54) A METHOD FOR DETERMINING THE EFFICACY OF TREATMENT WITH A COMBINATION OF DRUGS IN A SUBJECT DIAGNOSED WITH A DISEASE AND A METHOD FOR CLASSIFYING THE UTILITY OF DRUG COMBINATIONS IN TREATMENT OF SAID SUBJECT

(71) Applicant: VIVIA BIOTECH SL, Madrid (ES)

(72) Inventors: Julian GORROCHATEGUI GUILLEN, Madrid (ES); Jose Luis ROJAS RUDILLA, Madrid (ES); Joan BALLESTEROS NOBELL, Madrid (ES); Jesus VILLORIA MORILLO, Madrid (ES); Pilar HERNANDEZ DEL CAMPO, Madrid (ES); Daniel PRIMO RAMOS, Madrid (ES); Joaquin MARTINEZ LOPEZ, Madrid (ES); Pau MONTESINOS FERNÁNDEZ, Madrid (ES); David MARTÍNEZ CUADRÓN, Madrid (ES)

(21) Appl. No.: 17/284,674

(22) PCT Filed: Oct. 11, 2019
(86) PCT No.: PCT/EP2019/077637

§ 371 Date: Apr. 12, 2021

(30) Foreign Application Priority Data
Oct. 11, 2018 (EP) 18382726.0

(51) Int. Cl. G16C 20/30; G16C 20/70; G06F 17/11; G06F 17/18

(52) U.S. Cl. CPC G16C 20/30; G06F 17/18; G06F 17/11; G16C 20/70

(57) ABSTRACT

The present invention relates to a method and a system for determining the efficacy of treatment with a combination of drugs comprising a drug A and a drug B in a subject diagnosed with a disease, as well as to a method and a system for classifying the utility of drug combinations comprising a drug A and a drug B in treatment of a subject diagnosed with a disease. Also disclosed herein is a method for improving the accuracy in the estimation of synergism of a combination of drugs.

### FIELD OF THE INVENTION

The present invention relates to a method for determining the efficacy of treatment with a combination of drugs in a subject diagnosed with a disease. The present invention also relates to a method for classifying the utility of drug combinations in treatment of a subject diagnosed with a disease. The present invention also relates to a method for improving the accuracy in the estimation of synergism of a combination of drugs.

### BACKGROUND TO THE INVENTION

Drug combinations are commonly used in the treatment of disease, particularly cancers of the hematopoietic and lymphoid tissues such as acute myeloid leukemia. However, not all such drug combinations exhibit the same degree of synergy and, thus, the same efficacy against said disease, across all patients. Accordingly, it is a problem of the present invention to provide a method to determine the efficacy of treatment of a specific disease with a given combination of drugs in a given subject (i.e. the sensitivity or resistance of said disease to said combination of drugs in said subject), thereby providing an indication of the degree of synergy afforded by said combination of drugs against said disease in said subject.

In addition, since a variety of drug combinations are generally available for treatment of any given disease, it is a problem of the present invention to provide a method which classifies different drug combinations according to their efficacy or utility in treating said disease in a given subject, thereby offering a therapeutic guide to a clinician or the subject as to the drug combinations of most or least utility in treating said disease in said subject.

Furthermore, since clinical trials of a given combination of drugs against a given disease are required in order to achieve regulatory approval thereof, it is also a problem of the present invention to provide a companion diagnostic (CDx) biomarker for said combination of drugs, which allows subjects (patients) in which said disease is sensitive to treatment to be selected for inclusion in said trials.

It is a further problem of the present invention to ensure that each of the aforementioned methods exhibit high accuracy and reduced variability, so that each provides a robust analysis and improved correlation with clinical output, yet also minimises the amount of tissue sampled from each subject.

### BRIEF DESCRIPTION OF THE INVENTION

The present invention relates to a method for determining the efficacy of treatment with a combination of drugs comprising a drug A and a drug B in a subject diagnosed with a disease, wherein said method comprises the following steps:
(a) separating a tissue sample obtained from said subject into sub-samples;
(b) carrying out the steps of:
   (i) incubating a sub-sample for a time T of between 2 and 168 hours in the presence of said drug A at a concentration X; and
   (ii) repeating step (b)(i) an additional (N−1) times, each time with a different sub-sample using a value for X that is different from that used in previous repetitions of step (b)(i);
   wherein N is a whole number selected from between 5 and 10, inclusive;
   and
   (iii) incubating a sub-sample for said time Tin the presence of said drug B at a concentration Y; and
   (iv) repeating step (b)(iii) an additional (M−1) times, each time with a different sub-sample using a value for Y that is different from that used in previous repetitions of step (b)(iii);
   wherein M is a whole number selected from between 5 and 10, inclusive;
   and
   (v) incubating a sub-sample for said time T in the presence of a combination of drugs comprising said drug A and said drug B, wherein
      the concentration of said drug A is a concentration X corresponding to the concentration at the percentile value P_{Hα,A} from the distribution of X_{50,A} values obtained in said population of subjects each diagnosed with said disease, wherein percentile value P_{Hα,A} is calculated by the formula (A):

P_{Hα,A}=cos(α°)×H (A)
wherein:
   H corresponds to a reference percentile selected from the group of

$$10 , [ 10 + \frac{80}{R - 1} \times ( r - 1 ) ]$$

and 90, wherein:
   r is a whole number selected from between 2 and (R−1), inclusive
α is in degrees and is calculated from the formula:

$$α = \frac{90}{( W + 1 )} \times w$$

wherein:
   w is a whole number selected from between 1 and W, inclusive the concentration of said drug B is a concentration Y corresponding to the concentration at the percentile value P_{Hα,B} from the distribution of Y_{50,B} values obtained in said population of subjects each diagnosed with said disease, wherein percentile value P_{Hα,B} is calculated by the formula (B):

P_{Hα,B}=cos(90°−α°)×H (B)
(vi) repeating step (b)(v) an additional (R−1) times, each time with a different sub-sample using a value for H that is different from that used in previous repetitions of step (b)(v), and using the same value for w that is used in step (b)(v); and
(vii) repeating steps (b)(v) and (b)(vi) an additional (W−1) times, each time with a different sub-sample using a value for w that is different from that used in previous repetitions of steps (b)(v) and (b)(vi);
wherein;
   R is a whole number selected from between 3 and 10, inclusive;
   W is a whole number selected from between 3 and 10, inclusive,
and wherein:
   X_{50,A} is the concentration of drug A exerting half of the maximum activity in a subject, estimated according to step (e)(i), below;
   Y_{50,B} is the concentration of drug B exerting half of the maximum activity in a subject, estimated according to step (e)(i), below;
and
(viii) incubating a sub-sample for said time T;
(c) adding at least one marker to each sub-sample incubated in step (b) to identify at least one cell-type (CT) therein;
(d) counting the number of live cells (LCTi) of each cell-type identified in step (c) which remain after incubation of each sub-sample according to step (b);
(e) determining for each cell-type identified in step (c):
   (i) pharmacodynamic parameter values comprising an X_{50,A} value, a LCTi_{0,A} value, an E_{max,A} value, a γ_{A} value, a Y_{50,B} value, an LCTi_{0,B} value, an E_{max,B} value and/or a γ_{B} value, wherein:
      said X_{50,A}, LCTi_{0,A}, E_{max,A} and γ_{A} values are estimated from a single drug dose-response pharmacodynamic mixed effects non-linear population model determined by fitting the formula (I) to experimental values of LCTi counted according to step (d) after incubating sub-samples for each subject in a population according to steps (b)(i) and (b)(ii) obtained for each concentration X of drug A:

$$L C T i = {LCTi}_{0 , A} \times [ 1 - {E}_{max , A} \times \frac{{X}^{{γ}_{A}}}{{X}^{{γ}_{A}} + {X}_{50 , A}^{{γ}_{A}}} ] \qquad ( I )$$

said Y_{50,B}, LCTi_{0,B}, E_{max,B} and γ_{B} values are estimated from a single drug dose-response pharmacodynamic mixed effects non-linear population model determined by fitting the formula (II) to experimental values of LCTi counted according to step (d) after incubating sub-samples for each subject in said population according to steps (b)(iii) and (b)(iv) obtained for each concentration Y of drug B:

$$L C T i = L {CTi}_{0 , B} \times [ 1 - {E}_{max , B} \times \frac{{Y}^{{γ}_{B}}}{{Y}^{{γ}_{B}} + {Y}_{50 , B}^{{γ}_{B}}} ] \qquad ( II )$$

wherein said population comprises said subject and other subjects diagnosed with said disease;
wherein:
   X=concentration of drug A;
   X_{50,A} is the concentration of drug A exerting half of maximum activity;
   LCTi_{0,A} is the basal (pre-incubation) number of LCTi and is equal to the LCTi counted after incubating a sub-sample in the absence of a drug according to the step referred to in (b)(viii);
   E_{max,A}, is the maximum fractional decrease of LCTi_{0,A} caused by drug A;
   γ_{A} is the steepness of the LCTi vs concentration curve for drug A;
   Y=concentration of drug B;
   Y_{50,B} is the concentration of drug B exerting half of maximum activity;
   LCTi_{0,B} is the basal (pre-incubation) number of LCTi and is equal to the LCTi counted after incubating a sub-sample in the absence of a drug according to the step referred to in (b)(viii);
   E_{max,B}, is the maximum fractional decrease of LCTi_{0,B} caused by drug B;
   γ_{B} is the steepness of the LCTi vs concentration curve for drug B;
(ii) activity marker values comprising an AUC_{xy,A} value, an AUC_{xy,B} value, an α_{AB} value and/or a VUS_{AB} value, wherein:
   said AUC_{xy,A} value is calculated using the formula (III):

AUC_{xy,A}=AUC_{x,A}−A_{y:10-90,A} (III)
wherein:
said AUC_{x,A} value is the integral between two drug concentrations X′ and X″ of a function derived from formula (I) for the % survival after incubating sub-samples according to steps (b)(i) and (b)(ii) obtained for each concentration X of drug A, wherein LCTi_{0,A} is considered as 100% survival, and is calculated using the formula (IV):

$${AUC}_{x , A} = {\int }_{{X}^{′}}^{{X}^{″}} 100 \times [ 1 - {E}_{max , A} \times \frac{{X}^{{γ}_{A}}}{{X}^{{γ}_{A}} + {X}_{50 , A}^{{γ}_{A}}} ] dX \qquad ( IV )$$

wherein drug concentrations X′ and X″ correspond to the concentrations of the 20^{th} and 80^{th} percentiles of the X_{50,A} values obtained in said population of subjects each diagnosed with said disease, wherein X_{50,A} was calculated for each subject in said population according to steps (a) to (e)(i);
and
said A_{y:10-90,A} value is the surface from AUC_{x,A} that falls outside the 10% and 90% boundaries of the % survival, wherein LCTi_{0,A} is considered as 100% survival;
and
   said AUC_{xy,B} value is calculated using the formula (V):

AUC_{xy,B}=AUC_{x,B}−A_{y:10-90,B} (V)
wherein:
said AUC_{x,B} value is the integral between two drug concentrations Y′ and Y″ of a function derived from formula (II) for the % survival after incubating sub-samples according to steps (b)(iii) and (b)(iv) obtained for each concentration Y of drug B, wherein LCTi_{0,B} is considered as 100% survival, and is calculated using the formula (VI):

$${AUC}_{x , B} = {\int }_{{Y}^{′}}^{{Y}^{″}} 100 \times [ 1 - {E}_{max , B} \times \frac{{Y}^{{γ}_{B}}}{{Y}^{{γ}_{B}} + {Y}_{50 , B}^{{γ}_{B}}} ] dY \qquad ( VI )$$

wherein drug concentrations Y′ and Y″ correspond to the concentrations of the 20^{th} and 80^{th} percentiles of the Y_{50,B} values obtained in said population of subjects each diagnosed with said disease, wherein Y_{50,B} was calculated for each subject in said population according to steps (a) to (e)(i);
and
said A_{y:10-90,B} value is the surface from AUC_{x,B} that falls outside the 10% and 90% boundaries of the % survival, wherein LCTi_{0,A} is considered as 100% survival;
and
   said VUS_{AB} value is calculated using the formula (VII), wherein said VUS_{AB} value is the double integral between two drug concentrations X′ and X″ for drug A and two drug concentrations Y′ and Y″ for drug B of the model function of the natural log of LCTi counted after incubating sub-samples according to steps (b)(v) to (b)(vii), wherein LCTi_{0,A}=LCTi_{0,B} and is considered as 100% survival,

$$\begin{matrix} & ( VII ) \\ {VUS}_{AB} = {\int }_{{X}^{′}}^{{X}^{″}} {\int }_{{Y}^{′}}^{{Y}^{″}} 100 \times [ 1 - \frac{\begin{matrix}( {E}_{max , A} \times \frac{X}{X + {X}_{50 , A}} + {E}_{max , B} \times \frac{Y}{Y + {Y}_{50 , B}} + {α}_{AB} \times \\ {{E}_{max , A} \times {E}_{max , B} \times \frac{X}{X + {X}_{50 , A}} \times \frac{Y}{Y + {Y}_{50 , B}} )}^{{γ}_{int , AB}}\end{matrix}}{1 + {( \frac{X}{{X}_{50 , A}} + \frac{Y}{{Y}_{50 , B}} + {α}_{AB} \times \frac{X}{{X}_{50 , A}} \times \frac{Y}{{Y}_{50 , B}} )}^{{γ}_{int , AB}}} ] dX dY & \end{matrix}$$

wherein:
   drug concentrations X′ and X″ correspond to the concentrations of the 20th and 80th percentiles of the X_{50,A} values obtained in said population of subjects each diagnosed with said disease, wherein X_{50,A} was calculated for each subject in said population according to steps (a) to (e)(i);
   drug concentrations Y′ and Y″ correspond to the concentrations of the 20th and 80th percentiles of the Y_{50,B} values obtained in said population of subjects each diagnosed with said disease, wherein Y_{50,B} was calculated for each subject in said population according to steps (a) to (e)(i);
   E_{max,A}=maximum fractional decrease in LPC caused by drug A;
   E_{max,B}=maximum fractional decrease in LPC caused by drug B;
   X_{50,A}=EC₅₀ concentration of drug A exerting half of E_{max,A};
   Y_{50,B}=EC₅₀ concentration of drug B exerting half of E_{max,B};
   X=concentration of drug A;
   Y=concentration of drug B;

$${γ}_{int , AB} = {γ}_{A} \times \frac{\frac{X}{{X}_{50 , A}}}{\frac{X}{{X}_{50 , A}} + \frac{Y}{{Y}_{50 , B}}} + ( 1 - \frac{\frac{X}{{X}_{50 , A}}}{\frac{X}{{X}_{50 , A}} + \frac{Y}{{Y}_{50 , B}}} ) \times {γ}_{B}$$

wherein:
   γA=steepness of the LCTi vs concentration curve for drug A;
   γB=steepness of the LCTi vs concentration curve for drug B; and
α_{AB}=synergy parameter estimated from a two-drug surface interaction model determined by fitting the formula (VII′) to experimental values of LCTi counted according to step (d) after incubating sub-samples for said subject according to the steps referred to in (b)(i) and (b)(ii) obtained for each concentration X of drug A, the steps referred to in (b)(iii) and (b)(iv) obtained for each concentration Y of drug B, and the steps referred to in b(v), b(vi) and b(vii) obtained for each pair of concentrations of the combination of drug A and drug B:

$$\begin{matrix} & ( {VII}^{′} ) \\ LCTi = {LCTi}_{0 , AB} \times [ 1 - \frac{\begin{matrix}( {E}_{max , A} \times \frac{X}{X + {X}_{50 , A}} + {E}_{max , B} \times \frac{Y}{Y + {Y}_{50 , B}} + {α}_{AB} \times \\ {{E}_{max , A} \times {E}_{max , B} \times \frac{X}{X + {X}_{50 , A}} \times \frac{Y}{Y + {Y}_{50 , B}} )}^{{γ}_{int , AB}}\end{matrix}}{1 + {( \frac{X}{{X}_{50 , A}} + \frac{Y}{{Y}_{50 , B}} + {α}_{AB} \times \frac{X}{{X}_{50 , A}} \times \frac{Y}{{Y}_{50 , B}} )}^{{γ}_{int , AB}}} ] & \end{matrix}$$

(iii) normalized marker values comprising NAUC_{A}, NAUC_{B} and/or NVUS_{AB}, wherein:
   NAUC_{A} is a normalised value for AUC_{xy,A} which is calculated using the formula (VIII);

NAUC_{A}=100×AUC_{xy,A}/AUC_{max,A} (VIII)
NAUC_{B} is a normalised value for AUC_{xy,B} which is calculated using the formula (IX);

NAUC_{B}=100×AUC_{xy,B}/AUC_{max,B} (IX)
NVUS_{AB} is a normalised value for VUS_{AB} which is calculated using the formula (X);

NVUS_{AB}=100×VUS_{AB}/VUS_{max,AB} (X)
wherein:
   AUC_{max,A}=the maximum value for AUC_{xy,A} obtained in a population of subjects each diagnosed with said disease, wherein AUC_{xy,A} was calculated for each subject in said population according to steps (a) to (e)(ii);
   AUC_{max,B}=the maximum value for AUC_{xy,B} obtained in said population of subjects each diagnosed with said disease, wherein AUC_{xy,B} was calculated for each subject in said population according to steps (a) to (e)(ii); and
   VUS_{max,AB}=the maximum value for VUS_{AB} obtained in said population of subjects each diagnosed with said disease, wherein VUS_{AB} was calculated for each subject in said population according to steps (a) to (e)(ii);
(f) selecting:
   (i) the pharmacodynamic parameter value or values determined according to step (e)(i) for each subject in said population of subjects; and/or
   (ii) the activity marker value or values determined according to step (e)(ii) for each subject in said population of subjects; and/or
   (iii) normalized marker value or values determined according to step (e)(iii) for each subject in said population of subjects; and/or
   (iv) a clinical variable value or values for each subject in said population of subjects, which are dependent on clinical resistance or clinical sensitivity to said combination of drugs, whereby a value is dependent when the probability of said value being independent from clinical resistance or clinical sensitivity is less than or equal to 0.05;
(g) creating a response function using a generalized linear model or a generalized additive model for said combination of drugs for said population of subjects using at least one of the values which were selected in step (f), wherein the receiver operating characteristic curve derived from said model function has an area under the curve which is equal to or greater than 0.8, and the lower limit of the 95% confidence interval of said area under the curve is greater than 0.5;
(h) calculating a threshold limit of the response function created in step (g) from the point on said receiver operating characteristic curve:
   at which sensitivity and specificity are maximized and equal in value; or
   at which specificity is prioritized over sensitivity; or
   which is closest to the (1,0) coordinate plane.
(j) calculating a S/R value for said combination of drugs against said disease in said subject using the response function created in step (g) and:
   (i) the pharmacodynamic parameter value or values determined according to step (e)(i) for said subject; and/or
   (ii) the activity marker value or values determined according to step (e)(ii) for said subject; and/or
   (iii) the normalized marker value or values determined according to step (e)(iii) for said subject, and/or
   (iv) the clinical variable value or values for said subject, which are variables in said response function; and
(k) determining the efficacy of treatment of said disease in said subject with said combination of drugs by comparing the S/R value calculated in step (j) with the threshold limit calculated in step (h), wherein
   when said S/R value is equal to or greater than said threshold limit, said disease is sensitive to treatment with said combination of drugs in said subject; and
   when said S/R value is less than said threshold limit, said disease is resistant to treatment with said combination of drugs in said subject.

The present invention also relates to a system for determining the efficacy of treatment with a combination of drugs comprising a drug A and a drug B in a subject diagnosed with a disease, wherein said system comprises:

(a) means for carrying out the step of separating a tissue sample obtained from said subject into sub-samples;

(b) means for carrying out the steps of:
(i) incubating a sub-sample for a time T of between 2 and 168 hours in the presence of said drug A at a concentration X; and
(ii) repeating the step referred to in (b)(i) an additional (N−1) times, each time with a different sub-sample using a value for X that is different from that used in previous repetitions of the step referred to in (b)(i);
wherein N is a whole number selected from between 5 and 10, inclusive;
and
(iii) incubating a sub-sample for said time Tin the presence of said drug B at a concentration Y; and
(iv) repeating the step referred to in (b)(iii) an additional (M−1) times, each time with a different sub-sample using a value for Y that is different from that used in previous repetitions of the step referred to in (b)(iii);
wherein M is a whole number selected from between 5 and 10, inclusive;
and
(v) incubating a sub-sample for said time T in the presence of a combination of drugs comprising said drug A and said drug B, wherein
   the concentration of said drug A is a concentration X corresponding to the concentration at the percentile value P_{Hα,A} from the distribution of X_{50,A} values obtained in said population of subjects each diagnosed with said disease, wherein percentile value P_{Hα,A} is calculated by the formula (A):

P_{Hα,A}=cos(α°)×H (A)
wherein:
   H corresponds to a reference percentile selected from the group of

$$10 , \\ [ 10 + \frac{80}{R - 1} \times ( r - 1 ) ]$$

and 90, wherein:
   r is a whole number selected from between 2 and (R−1), inclusive
α is in degrees and is calculated from the formula:

$$α = \frac{90}{( W + 1 )} \times w$$

wherein:
   w is a whole number selected from between 1 and W, inclusive
the concentration of said drug B is a concentration Y corresponding to the concentration at the percentile value P_{Hα,B} from the distribution of Y_{50,B} values obtained in said population of subjects each diagnosed with said disease, wherein percentile value P_{Hα,B} is calculated by the formula (B):

P_{Hα,B}=cos(90°−α°)×H (B)
(vi) repeating the step referred to in (b)(v) an additional (R−1) times, each time with a different sub-sample using a value for H that is different from that used in previous repetitions of the step referred to in (b)(v), and using the same value for w that is used in the step referred to in (b)(v); and
(vii) repeating the steps referred to in (b)(v) and (b)(vi) an additional (W−1) times, each time with a different sub-sample using a value for w that is different from that used in previous repetitions of the steps referred to in (b)(v) and (b)(vi);
wherein;
   R is a whole number selected from between 3 and 10, inclusive;
   W is a whole number selected from between 3 and 10, inclusive,
and wherein:
   X_{50,A} is the concentration of drug A exerting half of the maximum activity in a subject, estimated according to the step referred to in (e)(i), below;
   Y_{50,B} is the concentration of drug B exerting half of the maximum activity in a subject, estimated according to the step referred to in (e)(i), below;
and
(viii) incubating a sub-sample for said time T;

(c) means for carrying out a step of adding at least one marker to each sub-sample incubated in the steps referred to in (b) to identify at least one cell-type (CT) therein;

(d) means for carrying out a step of counting the number of live cells (LCTi) of each cell type identified in step (c) which remain after incubation of each sub-sample according to the step referred to in (b);

(e) means for carrying out a step of determining for each cell-type identified in the step referred to in (c):
(i) pharmacodynamic parameter values comprising an X_{50,A} value, a LCTi_{0,A} value, an E_{max,A} value, a γ_{A} value, a Y_{50,B} value, an LCTi_{0,B} value, an E_{max,B} value and/or a γ_{B} value, wherein:
   said X_{50,A}, LCTi_{0,A}, E_{max,A} and γ_{A} values are estimated from a single drug dose-response pharmacodynamic mixed effects non-linear population model determined by fitting the formula (I) to experimental values of LCTi counted according to step (d) after incubating sub-samples for each subject in a population according to the steps referred to in (b)(i) and (b)(ii) obtained for each concentration X of drug A:

$$LCTi = {LCTi}_{0 , A} \times [ 1 - {E}_{max , A} \times \frac{{X}^{{γ}_{A}}}{{X}^{{γ}_{A}} + {X}_{50 , A}^{{γ}_{A}}} ] \qquad ( I )$$

said Y_{50,B}, LCTi_{0,B}, E_{max,B} and γ_{B} values are estimated from a single drug dose-response pharmacodynamic mixed effects non-linear population model determined by fitting the formula (II) to experimental values of LCTi counted according to step (d) after incubating sub-samples for each subject in said population according to the steps referred to in (b)(iii) and (b)(iv) obtained for each concentration Y of drug B:

$$LCTi = {LCTi}_{0 , B} \times [ 1 - {E}_{max , B} \times \frac{{Y}^{{γ}_{B}}}{{Y}^{{γ}_{B}} + {Y}_{50 , B}^{{γ}_{B}}} ] \qquad ( II )$$

wherein said population comprises said subject and other subjects diagnosed with said disease;
wherein:
   X=concentration of drug A;
   X_{50,A} is the concentration of drug A exerting half of maximum activity;
   LCTi_{0,A} is the basal (pre-incubation) number of LCTi and is equal to the LCTi counted after incubating a sub-sample in the absence of a drug according to the step referred to in (b)(viii);
   E_{max,A}, is the maximum fractional decrease of LCTi_{0,A} caused by drug A;
   γ_{A} is the steepness of the LCTi vs concentration curve for drug A;
   Y=concentration of drug B;
   Y_{50,B} is the concentration of drug B exerting half of maximum activity;
   LCTi_{0,B} is the basal (pre-incubation) number of LCTi and is equal to the LCTi counted after incubating a sub-sample in the absence of a drug according to the step referred to in (b)(viii);
   E_{max,B}, is the maximum fractional decrease of LCTi_{0,B} caused by drug B;
   γ_{B} is the steepness of the LCTi vs concentration curve for drug B
(ii) activity marker values comprising an AUC_{xy,A} value, an AUC_{xy,B} value, an α_{AB} value and/or a VUS_{AB} value, wherein:
   said AUC_{xy,A} value is calculated using the formula (III):

AUC_{xy,A}=AUC_{x,A}−A_{y:10-90,A} (III)
wherein:
said AUC_{x,A} value is the integral between two drug concentrations X′ and X″ of a function derived from formula (I) for the % survival after incubating sub-samples according to the steps referred to in (b)(i) and (b)(ii) obtained for each concentration of drug A wherein LCTi_{0,A} is considered as 100% survival, and is calculated using the formula (IV):

$${AUC}_{x , A} = {\int }_{{X}^{′}}^{{X}^{″}} 100 \times [ 1 - {E}_{max , A} \times \frac{{X}^{{γ}_{A}}}{{X}^{{γ}_{A}} + {X}_{50 , A}^{{γ}_{A}}} ] dX \qquad ( IV )$$

wherein drug concentrations X′ and X″ correspond to the concentrations of the 20^{th} and 80^{th} percentiles of the X_{50,A} values obtained in said population of subjects each diagnosed with said disease, wherein X_{50,A} was calculated for each subject in said population according to steps (a) to (e)(i);
and
said A_{y:10-90,A} value is the surface from AUC_{x,A} that falls outside the 10% and 90% boundaries of the % survival, wherein LCTi_{0,A} is considered as 100% survival;
and
said AUC_{xy,B} value is calculated using the formula (V):

AUC_{xy,B}=AUC_{x,B}−A_{y:10-90,B} (V)
wherein:
said AUC_{x,B} value is the integral between two drug concentrations Y′ and Y″ of a function derived from formula (II) for the % survival after incubating sub-samples according to the steps referred to in (b)(iii) and (b)(iv) obtained for each concentration of drug B, wherein LCTi_{0,B} is considered as 100% survival, and is calculated using the formula (VI):

$${AUC}_{x , B} = {\int }_{{Y}^{′}}^{{Y}^{″}} 100 \times [ 1 - {E}_{max , B} \times \frac{{X}^{{γ}_{B}}}{{X}^{{γ}_{B}} + {X}_{50 , B}^{{γ}_{B}}} ] dY \qquad ( VI )$$

wherein drug concentrations Y′ and Y″ correspond to the concentrations of the 20^{th} and 80^{th} percentiles of the Y_{50,B} values obtained in said population of subjects each diagnosed with said disease, wherein Y_{50,B} was calculated for each subject in said population according to steps (a) to (e)(i);
and
said A_{y:10-90,B} value is the surface from AUC_{x,B} that falls outside the 10% and 90% boundaries of the % survival, wherein LCTi_{0,A} is considered as 100% survival;
and
said VUS_{AB} value is calculated using the formula (VII), wherein said VUS_{AB} value is the double integral between two drug concentrations X′ and X″ for drug A and two drug concentrations Y′ and Y″ for drug B of the model function of the natural log of LCTi counted after incubating sub-samples according to the steps referred to in (b)(v) to (b)(vii), wherein LCTi_{0,A}=LCTi_{0,B} and is considered as 100% survival, and is calculated using the formula (VII),

$$\begin{matrix} & ( VII ) \\ {VUS}_{AB} = {\int }_{{X}^{′}}^{{X}^{″}} {\int }_{{Y}^{′}}^{{Y}^{″}} 100 \times [ 1 - \frac{\begin{matrix}( {E}_{max , A} \times \frac{X}{X + {X}_{50 , A}} + {E}_{max , B} \times \frac{Y}{Y + {Y}_{50 , B}} + {α}_{AB} \times \\ {{E}_{max , A} \times {E}_{max , B} \times \frac{X}{X + {X}_{50 , A}} \times \frac{Y}{Y + {Y}_{50 , B}} )}^{{γ}_{int , AB}}\end{matrix}}{1 + {( \frac{X}{{X}_{50 , A}} + \frac{Y}{{Y}_{50 , B}} + {α}_{AB} \times \frac{X}{{X}_{50 , A}} \times \frac{Y}{{Y}_{50 , B}} )}^{{γ}_{int , AB}}} ] dX dY & \end{matrix}$$

wherein:
   drug concentrations X′ and X″ correspond to the concentrations of the 20^{th} and 80^{th} percentiles of the X_{50,A} values obtained in said population of subjects each diagnosed with said disease, wherein X_{50,A} was calculated for each subject in said population according to steps (a) to (e)(i);
   drug concentrations Y′ and Y″ correspond to the concentrations of the 20^{th} and 80^{th} percentiles of the Y_{50,B} values obtained in said population of subjects each diagnosed with said disease, wherein Y_{50,B} was calculated for each subject in said population according to steps (a) to (e)(i);
   E_{max,A}=maximum fractional decrease in LPC caused by drug A;
   E_{max,B}=maximum fractional decrease in LPC caused by drug B;
   X_{50,A}=EC₅₀ concentration of drug A exerting half of E_{max,A};
   Y_{50,B}=EC₅₀ concentration of drug B exerting half of E_{max,B};
   X=concentration of drug A;
   Y=concentration of drug B;

$${γ}_{int , AB} = {γ}_{A} \times \frac{\frac{X}{{X}_{50 , A}}}{\frac{X}{{X}_{50 , A}} + \frac{Y}{{Y}_{50 , B}}} + ( 1 - \frac{\frac{X}{{X}_{50 , A}}}{\frac{X}{{X}_{50 , A}} + \frac{Y}{{Y}_{50 , B}}} ) \times {γ}_{B}$$

wherein:
γA=steepness of the LCTi vs concentration curve for drug A;
γB=steepness of the LCTi vs concentration curve for drug B; and
α_{AB}=synergy parameter estimated from a two-drug surface interaction model determined by fitting the formula (VII′) to experimental values of LCTi counted according to the step referred to in (d) after incubating sub-samples for said subject according to the steps referred to in (b)(i) and (b)(ii) obtained for each concentration X of drug A, the steps referred to in (b)(iii) and (b)(iv) obtained for each concentration Y of drug B, and the steps referred to in b(v), b(vi) and b(vii) obtained for each pair of concentrations of the combination of drug A and drug B:

$$\begin{matrix} & ( {VII}^{′} ) \\ LCTi = {LCTi}_{0 , AB} \times [ 1 - \frac{\begin{matrix}( {E}_{max , A} \times \frac{X}{X + {X}_{50 , A}} + {E}_{max , B} \times \frac{Y}{Y + {Y}_{50 , B}} + {α}_{AB} \times \\ {{E}_{max , A} \times {E}_{max , B} \times \frac{X}{X + {X}_{50 , A}} \times \frac{Y}{Y + {Y}_{50 , B}} )}^{{γ}_{int , AB}}\end{matrix}}{1 + {( \frac{X}{{X}_{50 , A}} + \frac{Y}{{Y}_{50 , B}} + {α}_{AB} \times \frac{X}{{X}_{50 , A}} \times \frac{Y}{{Y}_{50 , B}} )}^{{γ}_{int , AB}}} ] & \end{matrix}$$

(iii) normalized marker values comprising NAUC_{A}, NAUC_{B} and/or NVUS_{AB}, wherein:
   NAUC_{A} is a normalised value for AUC_{xy,A} which is calculated using the formula (VIII);

NAUC_{A}=100×AUC_{xy,A}/AUC_{max,A} (VIII)
NAUC_{B} is a normalised value for AUC_{xy,B} which is calculated using the formula (IX);

NAUC_{B}=100×AUC_{xy,B}/AUC_{max,B} (IX)
NVUS_{AB} is a normalised value for VUS_{AB} which is calculated using the formula (X);

NVUS_{AB}=100×VUS_{AB}/VUS_{max,AB} (X)
wherein:
   AUC_{max,A}=the maximum value for AUC_{xy,A} obtained in a population of subjects each diagnosed with said disease, wherein AUC_{xy,A} was calculated for each subject in said population according to the step referred to in (a) to (e)(ii);
   AUC_{max,B}=the maximum value for AUC_{xy,B} obtained in said population of subjects each diagnosed with said disease, wherein AUC_{xy,B} was calculated for each subject in said population according to the step referred to in (a) to (e)(ii); and
   VUS_{max,AB}=the maximum value for VUS_{AB} obtained in said population of subjects each diagnosed with said disease, wherein VUS_{AB} was calculated for each subject in said population according to the step referred to in (a) to (e)(ii);

(f) means for carrying out a step of selecting:
(i) the pharmacodynamic parameter value or values determined according the step referred to in (e)(i) for each subject in said population of subjects; and/or
(ii) the activity marker value or values determined according the step referred to in (e)(ii) for each subject in said population of subjects; and/or
(iii) normalized marker value or values determined according the step referred to in (e)(iii) for each subject in said population of subjects, and/or
(iv) a clinical variable value or values for each subject in said population of subjects, which are dependent on clinical resistance or clinical sensitivity to said combination of drugs, whereby a value is dependent when the probability of said value being independent from clinical resistance or clinical sensitivity is less than or equal to 0.05;

(g) means for carrying out a step of creating a response function using a generalized linear model or a generalized additive model for said combination of drugs for said population of subjects using at least one of the values which were selected in the step referred to in (f), wherein the receiver operating characteristic curve derived from said model function has an area under the curve which is equal to or greater than 0.8, and the lower limit of the 95% confidence interval of said area under the curve is greater than 0.5;

(h) means for carrying out a step of calculating a threshold limit of the response function created in the step referred to in (g) from the point on said receiver operating characteristic curve:
at which sensitivity and specificity are maximized and equal in value; or
at which specificity is prioritized over sensitivity; or
which is closest to the (1,0) coordinate plane.

(j) means for carrying out a step of calculating a S/R value for said combination of drugs against said disease in said subject using the response function created in the step referred to in (g) and:
(i) the pharmacodynamic parameter value or values determined according to the step referred to in (e)(i) for said subject; and/or
(ii) the activity marker value or values determined according to the step referred to in (e)(ii) for said subject; and/or
(iii) the normalized marker value or values determined according to the step referred to in (e)(iii) for said subject, and/or
(iv) the clinical variable value or values for said subject, which are variables in said response function; and

(k) means for carrying out a step of determining the efficacy of treatment of said disease in said subject with said combination of drugs by comparing the S/R value calculated in the step referred to in (j) with the threshold limit calculated in the step referred to in (h), wherein
when said S/R value is equal to or greater than said threshold limit, said disease is sensitive to treatment with said combination of drugs in said subject; and
when said S/R value is less than said threshold limit, said disease is resistant to treatment with said combination of drugs in said subject.

The present invention also relates to a method for classifying the utility of combinations of drugs, each comprising a drug A and a drug B, in treatment of a subject diagnosed with a disease, wherein said method comprises the following steps:
(a) separating a tissue sample obtained from said subject into sub-samples;
(b) carrying out the steps of:
   (i) incubating a sub-sample for a time T of between 2 and 168 hours in the presence of said drug A at a concentration X; and
   (ii) repeating step (b)(i) an additional (N−1) times, each time with a different sub-sample using a value for X that is different from that used in previous repetitions of step (b)(i);
   wherein N is a whole number selected from between 5 and 10, inclusive;
   and
   (iii) incubating a sub-sample for said time Tin the presence of said drug B at a concentration Y; and
   (iv) repeating step (b)(iii) an additional (M−1) times, each time with a different sub-sample using a value for Y that is different from that used in previous repetitions of step (b)(iii);
   wherein M is a whole number selected from between 5 and 10, inclusive;
   and
   (v) incubating a sub-sample for said time T in the presence of a combination of drugs comprising said drug A and said drug B, wherein
      the concentration of said drug A is a concentration X corresponding to the concentration at the percentile value P_{Hα,A} from the distribution of X_{50,A} values obtained in said population of subjects each diagnosed with said disease, wherein percentile value P_{Hα,A} is calculated by the formula (A):

P_{Hα,A}=cos(α°)×H (A)
wherein:
   H corresponds to a reference percentile selected from the group of

$$10 , \\ [ 10 + \frac{80}{R - 1} \times ( r - 1 ) ]$$

and 90, wherein:
   r is a whole number selected from between 2 and (R−1), inclusive
α is in degrees and is calculated from the formula:

$$α = \frac{90}{( W + 1 )} \times w$$

wherein:
   w is a whole number selected from between 1 and W, inclusive the concentration of said drug B is a concentration Y corresponding to the concentration at the percentile value P_{Hα,B} from the distribution of Y_{50,B} values obtained in said population of subjects each diagnosed with said disease, wherein percentile value P_{Hα,B} is calculated by the formula (B):

P_{Hα,B}=cos(90°−α°)×H (B)
(vi) repeating step (b)(v) an additional (R−1) times, each time with a different sub-sample using a value for H that is different from that used in previous repetitions of step (b)(v), and using the same value for w that is used in step (b)(v); and
(vii) repeating steps (b)(v) and (b)(vi) an additional (W−1) times, each time with a different sub-sample using a value for w that is different from that used in previous repetitions of steps (b)(v) and (b)(vi);
wherein;
   R is a whole number selected from between 3 and 10, inclusive;
   W is a whole number selected from between 3 and 10, inclusive,
and wherein:
   X_{50,A} is the concentration of drug A exerting half of the maximum activity in a subject, estimated according to step (e)(i), below;
   Y_{50,B} is the concentration of drug B exerting half of the maximum activity in a subject, estimated according to step (e)(i), below;
and
(viii) incubating a sub-sample for said time T;
(c) adding at least one marker to each sub-sample incubated in step (b) to identify at least one cell-type (CT) therein;
(d) counting the number of live cells (LCTi) of each cell-type identified in step (c) which remain after incubation of each sub-sample according to step (b);
(e) determining for each cell-type identified in step (c):
   (i) pharmacodynamic parameter values comprising at least one pharmacodynamic parameter value for drug A and/or at least one pharmacodynamic parameter value for drug B,
   wherein:
      each pharmacodynamic parameter value for drug A is estimated from a single drug dose-response pharmacodynamic mixed effects non-linear population model by fitting a formula to experimental values of LCTi counted according to step (d) after incubating sub-samples for each subject in the population according to steps (b)(i) and (ii); and
      each pharmacodynamic parameter value for drug B is estimated from a single drug dose-response pharmacodynamic mixed effects non-linear population model by fitting a formula to experimental values of LCTi counted according to step (d) after incubating sub-samples for each subject in the population according to steps (b)(iii) and (iv),
      wherein said population comprises said subject and other subjects diagnosed with said disease;
   (ii) activity marker values comprising at least one activity marker value for drug A, at least one activity marker value for drug B and/or at least one activity marker value for drugs A and B, wherein:
      each activity marker value for drug A is calculated from said pharmacodynamic parameter value or values for drug A estimated in step (e)(i),
      each activity marker value for drug B is calculated from said pharmacodynamic parameter value or values for drug B estimated in step (e)(i),
      each activity marker value for drugs A and B is calculated from a specific model made by fitting a formula to said pharmacodynamic parameter value or values for drug A and said pharmacodynamic parameter value or values for drug B which are estimated in step (e)(i), as well as to experimental values of LCTi counted according to step (d) after incubating sub-samples for each subject in the population according to steps (b)(v) to (vii);
      and
   (iii) normalized marker values comprising at least one normalized marker value for drug A, at least one normalized marker value for drug B and/or at least one normalized marker value for drugs A and B, wherein:
      each normalized marker value for drug A is calculated from the ratio of each activity marker value for drug A that is calculated in step (e)(ii) relative to a corresponding value from the distribution of said activity marker value for said population;
      each normalized marker value for drug B is calculated from the ratio of each activity marker value for drug B that is calculated in step (e)(ii) relative to a corresponding value from the distribution of said activity marker value for drug B for said population;
      each normalized marker value for drugs A and B is calculated from the ratio of each activity marker value for drugs A and B that is calculated in step (e)(ii) relative to a corresponding value from the distribution of said activity marker value for drugs A and B for said population;
(f) selecting:
   (i) the pharmacodynamic parameter value or values determined according to step (e)(i) for each subject in said population of subjects; and/or
   (ii) the activity marker value or values determined according to step (e)(ii) for each subject in said population of subjects; and/or
   (iii) normalized marker value or values determined according to step (e)(iii) for each subject in said population of subjects, and/or
   (iv) a clinical variable value or values for each subject in said population of subjects, which are dependent on clinical resistance or clinical sensitivity to said combination of drugs, whereby a value is dependent when the probability of said value being independent from clinical resistance or clinical sensitivity is less than or equal to 0.05;
(g′) calculating a score S for treatment of said subject with said drug A and said drug B, wherein said score corresponds with or is calculated using at least one of the values which were selected in step (f);
(h′) carrying out steps (b) to (g′) for each combination of drugs to be classified;
and
(j′) classifying each combination of drugs using the score determined in steps (g′) and (h′), whereby a combination of drugs having a score of:
   (i) greater than 80 is assigned to classification category I having a classification value of 2;
   (ii) less than or equal to 80 and greater than 60 is assigned to classification category II having a classification value of 1;
   (iii) less than or equal to 60 and greater than 40 is assigned to classification category III having a classification value of 0;
   (iv) less than or equal to 40 and greater than 20 is assigned to classification category IV having a classification value of −1; or
   (v) less than or equal to 20 is assigned to classification category V having a classification value of −2,
   whereby:
   each combination of drugs which is assigned to a classification category having a positive classification value or a classification value of zero is of highest utility in treatment of said disease in said subject; and
   each combination of drugs which is assigned to classification category having a negative classification value is of lowest utility in treatment of said disease in said subject.

Analogously, the present invention also relates to a system for classifying the utility of combinations of drugs, each comprising a drug A and a drug B, in treatment of a subject diagnosed with a disease, wherein said system comprises:

(a) means for carrying out the step of separating a tissue sample obtained from said subject into sub-samples;

(b) means for carrying out the steps of:
(i) incubating a sub-sample for a time T of between 2 and 168 hours in the presence of said drug A at a concentration X; and
(ii) repeating the step referred to in (b)(i) an additional (N−1) times, each time with a different sub-sample using a value for X that is different from that used in previous repetitions of the step referred to in (b)(i);
   wherein N is a whole number selected from between 5 and 10, inclusive;
   and
(iii) incubating a sub-sample for said time Tin the presence of said drug B at a concentration Y; and
(iv) repeating the step referred to in (b)(iii) an additional (M−1) times, each time with a different sub-sample using a value for Y that is different from that used in previous repetitions of the step referred to in (b)(iii);
   wherein M is a whole number selected from between 5 and 10, inclusive;
   and
(v) incubating a sub-sample for said time T in the presence of a combination of drugs comprising said drug A and said drug B, wherein
   the concentration of said drug A is a concentration X corresponding to the concentration at the percentile value P_{Hα,A} from the distribution of X_{50,A} values obtained in said population of subjects each diagnosed with said disease, wherein percentile value P_{Hα,A} is calculated by the formula (A):

P_{Hα,A}=cos(α°)×H (A)
wherein:
   H corresponds to a reference percentile selected from the group of

$$10 , [ 10 + \frac{80}{R - 1} \times ( r - 1 ) ]$$

and 90, wherein:
r is a whole number selected from between 2 and (R−1), inclusive
α is in degrees and is calculated from the formula:

$$α = \frac{90}{( W + 1 )} \times w$$

wherein:
w is a whole number selected from between 1 and W, inclusive
the concentration of said drug B is a concentration Y corresponding to the concentration at the percentile value P_{Hα,B} from the distribution of Y_{50,B} values obtained in said population of subjects each diagnosed with said disease, wherein percentile value P_{Hα,B} is calculated by the formula (B):

P_{Hα,B}=cos(90°−α°)×H (B)
(vi) repeating the step referred to in (b)(v) an additional (R−1) times, each time with a different sub-sample using a value for H that is different from that used in previous repetitions of the step referred to in (b)(v), and using the same value for w that is used in the step referred to in (b)(v); and
(vii) repeating the steps referred to in (b)(v) and (b)(vi) an additional (W−1) times, each time with a different sub-sample using a value for w that is different from that used in previous repetitions of the steps referred to in (b)(v) and (b)(vi);
   wherein;
      R is a whole number selected from between 3 and 10, inclusive;
      W is a whole number selected from between 3 and 10, inclusive,
   and wherein:
      X_{50,A} is the concentration of drug A exerting half of the maximum activity in a subject, estimated according to the step referred to in (e)(i), below;
      Y_{50,B} is the concentration of drug B exerting half of the maximum activity in a subject, estimated according to the step referred to in (e)(i), below;
   and
(viii) incubating a sub-sample for said time T;

(c) means for carrying out a step of adding at least one marker to each sub-sample incubated in the steps referred to in (b) to identify at least one cell-type (CT) therein;

(d) means for carrying out a step of counting the number of live cells (LCTi) of each cell-type identified in the step referred to in (c) which remain after incubation of each sub-sample according to the steps referred to in (b);

(e) means for carrying out a step of determining for each cell-type identified in the step referred to in (c):
(i) pharmacodynamic parameter values comprising at least one pharmacodynamic parameter value for drug A and/or at least one pharmacodynamic parameter value for drug B, wherein:
   each pharmacodynamic parameter value for drug A is estimated from a single drug dose-response pharmacodynamic mixed effects non-linear population model by fitting a formula to experimental values of LCTi counted according to step (d) after incubating sub-samples for each subject in the population according to steps (b)(i) and (ii); and
   each pharmacodynamic parameter value for drug B is estimated from a single drug dose-response pharmacodynamic mixed effects non-linear population model by fitting a formula to experimental values of LCTi counted according to step (d) after incubating sub-samples for each subject in the population according to steps (b)(iii) and (iv), wherein said population comprises said subject and other subjects diagnosed with said disease;
(ii) activity marker values comprising at least one activity marker value for drug A, at least one activity marker value for drug B and/or at least one activity marker value for drugs A and B, wherein:
   each activity marker value for drug A is calculated from said pharmacodynamic parameter value or values for drug A estimated in step (e)(i),
   each activity marker value for drug B is calculated from said pharmacodynamic parameter value or values for drug B estimated in step (e)(i),
   each activity marker value for drugs A and B is calculated from a specific model made by fitting a formula to said pharmacodynamic parameter value or values for drug A and said pharmacodynamic parameter value or values for drug B which are estimated in step (e)(i), as well as to experimental values of LCTi counted according to step (d) after incubating sub-samples for each subject in the population according to steps (b)(v) to (vii);
and
(iii) normalized marker values comprising at least one normalized marker value for drug A, at least one normalized marker value for drug B and/or at least one normalized marker value for drugs A and B, wherein:
   each normalized marker value for drug A is calculated from the ratio of each activity marker value for drug A that is calculated in step (e)(ii) relative to a corresponding value from the distribution of said activity marker value for said population;
   each normalized marker value for drug B is calculated from the ratio of each activity marker value for drug B that is calculated in step (e)(ii) relative to a corresponding value from the distribution of said activity marker value for drug B for said population;
   each normalized marker value for drugs A and B is calculated from the ratio of each activity marker value for drugs A and B that is calculated in step (e)(ii) relative to a corresponding value from the distribution of said activity marker value for drugs A and B for said population;

(f) means for carrying out a step of selecting:
(i) the pharmacodynamic parameter value or values determined according the step referred to in (e)(i) for each subject in said population of subjects; and/or
(ii) the activity marker value or values determined according the step referred to in (e)(ii) for each subject in said population of subjects; and/or
(iii) normalized marker value or values determined according the step referred to in (e)(iii) for each subject in said population of subjects, and/or
(iv) a clinical variable value or values for each subject in said population of subjects, which are dependent on clinical resistance or clinical sensitivity to said combination of drugs, whereby a value is dependent when the probability of said value being independent from clinical resistance or clinical sensitivity is less than or equal to 0.05;

(g′) means for carrying out a step of calculating a score S for treatment of said subject with said drug A and said drug B, wherein said score corresponds with or is calculated using at least one of the values which were selected in the step referred to in (f);

(h′) means for carrying out the steps referred to in (b) to (g′) for each combination of drugs to be classified;
and

(j) means for carrying out the step of classifying each combination of drugs using the score determined in the steps referred to in (g′) and (h′), whereby a combination of drugs having a score of:
(i) greater than 80 is assigned to classification category I having a classification value of 2;
(ii) less than or equal to 80 and greater than 60 is assigned to classification category II having a classification value of 1;
(iii) less than or equal to 60 and greater than 40 is assigned to classification category III having a classification value of 0;
(iv) less than or equal to 40 and greater than 20 is assigned to classification category IV having a classification value of −1; or
(v) less than or equal to 20 is assigned to classification category V having a classification value of −2,
whereby:
each combination of drugs which is assigned to a classification category having a positive classification value or a classification value of zero is of highest utility in treatment of said disease in said subject; and
each combination of drugs which is assigned to classification category having a negative classification value is of lowest utility in treatment of said disease in said subject.

Therefore, the present invention also relates to use of the method or the system for determining the efficacy of treatment with a combination of drugs comprising a drug A and a drug B in a subject diagnosed with a disease, in determining the efficacy of treatment with said combination of drugs in said subject diagnosed with said disease.

Analogously, the present invention also relates to use of the method or the system for classifying the utility of drug combinations each comprising a drug A and a drug B in treatment of a subject diagnosed with a disease, in determining the combinations of drugs which are classified highest in utility for the treatment of said disease in said subject diagnosed with said disease.

The method and system of the present invention for determining the efficacy of treatment with a combination of drugs comprising a drug A and a drug B in a subject diagnosed with a disease may further comprise means for prescribing a plan of care to a subject, wherein said plan of care prescribes said combination of drugs when said disease is determined to be sensitive to treatment with said combination of drugs in said subject. Thus, the present invention also relates to use of said method or system in prescribing said plan of care to said subject.

Analogously, the method and system of the present invention for classifying the utility of drug combinations each comprising a drug A and a drug B in treatment of a subject diagnosed with a disease may further comprise prescribing a plan of care to said subject, wherein said plan of care prescribes a combination of drugs selected from the combinations of drugs which are classified highest in utility for the treatment of said disease in said subject. Thus, the present invention also relates to use of said method or system in prescribing said plan of care to said subject.

Moreover, the present invention relates to a method of treatment of a subject diagnosed with a disease, comprising administration of a combination of drugs to said subject, wherein said disease is determined to be sensitive to treatment with said combination of drugs in said subject according to the method or the system for determining the efficacy of treatment with a combination of drugs comprising a drug A and a drug B in a subject diagnosed with a disease.

Analogously, the present invention relates to a method of treatment of a subject diagnosed with a disease, comprising administration of a combination of drugs selected from the combinations of drugs which are classified highest in utility for the treatment of said disease in said subject according to the method or system for classifying the utility of drug combinations each comprising a drug A and a drug B in treatment of a subject diagnosed with a disease.

The present invention also relates to use of the method or the system for determining the efficacy of treatment with a combination of drugs comprising a drug A and a drug B in a subject diagnosed with a disease, for determining whether a given subject from a population of subjects each diagnosed with a disease is suitable for inclusion in a clinical trial involving treatment with a combination of drugs comprising a drug A and a drug B, wherein:
when the disease is determined to be sensitive to treatment with said combination of drugs in said subject, said subject is selected for inclusion in said clinical trial; and
when the disease is determined to be resistant to treatment with said combination of drugs in said subject, said subject is not selected for inclusion in said clinical trial.

Analogously, the present invention relates to use of the method or the system for classifying the utility of drug combinations each comprising a drug A and a drug B in treatment of a subject diagnosed with a disease, for determining whether a given subject from a population of subjects each diagnosed with a disease is suitable for inclusion in a clinical trial involving treatment with a combinations of drugs comprising a drug A and a drug B, wherein:
when said combinations of drugs is classified highest in utility in treatment of said disease in said subject, said subject is selected for inclusion in said clinical trial; and
when said combinations of drugs is classified highest in utility in treatment of said disease in said subject, said subject is not selected for inclusion in said clinical trial.

Lastly, the present invention also relates to the aforementioned method and system for classifying the utility of combinations of drugs, each comprising a drug A and a drug B, in treatment of a subject diagnosed with a disease, wherein:
said disease is acute myeloid leukemia;
drug A is selected from the group of idarubicin, cytarabine, fludarabine, etoposide, thioguanine, clofarabine, cladribine, daunorubicin, mitoxantrone and amsacrine, drug B is subsequently selected from the remaining group of drugs after drug A has been selected therefrom and, optionally, the combination of drugs comprises drug C selected from the remaining group of drugs listed under drug A after drugs A and B have been selected therefrom
a combination of drugs having a score of greater than 80, more preferably greater than 85, even more preferably greater than 90, is assigned to classification category I having a classification value of 2 and is of highest utility in treatment of said disease in said subject,

whereby a combination of drugs belonging to this classification category, as determined by said method and system for classifying the utility of combinations of drugs, is selected in a plan of care for prescription in a method of treatment.

### DESCRIPTION OF THE FIGURES

FIG. 1. Sequential workflow of experimental (panel A) and analytical (panels B and C) methods applied in the study. Whole bone marrow samples [A] were incubated preserving the native microenvironment with drugs and drugs mixtures. Automated flow cytometry [B] followed by dot-plots analysis [C] allowed the counting after incubation of Live Pathologic Cells (LPC) at control wells and wells with increasing drugs concentrations. Data was uploaded into the LIMS system. Response vs drug concentration relationships were analyzed through non-linear mixed effect population modelling [E]. Predicted pharmacodynamic profiles were integrated between the 80% confidence interval of the individual estimate of EC₅₀ in order to calculate the area under the curve (AUC) used as a single activity marker. Similarly, a double integration of the two-variables interaction surface function allows the calculation of the volume under the surface (VUS) that is effected by the sign (synergy or antagonism) of the interaction [F]. Correlation of activity markers with clinical output was analyzed by Generalized Additive Models (GAM) [G] and ROC curves [H].

FIG. 2. General description of the system of the invention. Test conditions and parameters must be predefined and registered in the database. Based on that information, input data, coming out from cytometer files and user manual entry, is processed through R Runtime scripts which retrieve data from the database, pass it through to NONMEM and get back results, that are post-processed to final treatments score. Results, as well as every intermediate data and processes parameters are stored in the database ensuring full traceability. Results are output in proper html report.

FIG. 3. Flow chart showing the sequential steps in the process (straight line) as well as side data transferences (dotted lines). Database collects all the processed data and test results as well as required settings and operational parameters. Results Processing Templates (RPT) compile the required parameter settings for every test definition. Labelled (A-G) subprocesses are described in detail further on.

FIG. 4. Population models are built from scratch using a minimum number of samples (30) or when the accumulated samples number for a model is more than the 20% of the samples used to build the previous model. A valid model should have an Objective Function Value (OFV) at least 4 points lower than the previous model but having biological sensible model parameters and meet several conditions like Inter-Patient Variability (IPV), Standard Error (SE) and Quality Control check values. If not, then the model should be rebuilt again after 10 new samples. When the new model conditions are met then all the new model data is stored in the database to be used next time.

FIG. 5. Results Processing Template for PM tests compile all required parameters for running the whole process. Most of them are derived from the experimental design in use. Thus, protocol ID is the first parameter fixed in the RPT. Linked to it are the drugs and combinations tested as well as the plate format. RPT's also include definitions for QC test criteria, modelling execution parameters, post-processing and scoring calculations, clinical treatments evaluated and reports design.

FIG. 6. Models are executed in sequential loops for all drugs and combos tested and defined in a protocol and included in the corresponding RPT. Whereas for single drugs (panel A) where population mixed effect models are applied using R runtime and NONMEM software, the effect of combinations of drugs (panel B) are studied using individual interaction surface models which are fed with parameters from single drugs population models. Confidence intervals (CI) are calculated in the case of single drugs by bootstrapping from 1000 simulations using NONMEN software and single drugs modelling output results. For the interaction parameter, CI are calculated by non-parametric bootstrapping (1000).

FIG. 7. Single drugs model functions are integrated among specific concentrations values bounds to generate the Area Under Curve (AUC) which is used as the optimized activity marker. Integration limits are set based on statistical analysis of stored results distribution. Same limits are used for the Volume Under the Surface (VUS) calculation that follows a double integration of the two-variable function that define the surface interaction model. Upper and lower limits (UL and LL) are calculated for the extreme values of the Confidence Intervals estimated for the model parameters.

FIG. 8. Single drugs and combinations results are input to calculate the score of every treatment included in the test definition of the method for classifying the utility of drug combinations comprising a drug A and a drug B in treatment of a subject diagnosed with a disease. The score is therefore used to classify treatments.

FIG. 9. System is designed to automatically check for required upgrades. These upgrades will be set after executing new models or after applying changes in experimental conditions or report format. New models may be required for new drugs or combinations included in the test or dynamically in existing models by reaching a critical number of samples (NT), higher than the defined threshold (N u) to rebuild the model.

FIG. 10. The application includes a report designer to build up report templates. One or many templates may be included in an RPT and applied to sample results. Validation is a critical step carried out by an authorized user before publishing the report on a web server. Access to that server will be controlled and granted to authenticated end users.

FIG. 11. Visual predictive check of the population pharmacokinetic models of (panel A) cytarabine [In(Live Tumor Cells) vs. Cytarabine concentration] and (panel B) idarubicin [In(Live Tumor Cells) vs. Idarubicin concentration]. Open circles are the observed data points, the solid and dashed red lines are, respectively, the median and the 5-95th percentiles of the observed distribution of In(cells), and the semitransparent red and blue bands represent, respectively, the simulation-based 95% confidence intervals for the median and 5-95th percentiles of the estimated population distribution of In(cells).

FIG. 12. Regression hyperplane of the predicted probability of resistance (i.e. probability of being non-responder) over the AUCs of cytarabine and idarubicin for identifying the ex vivo variables associated with hematological response. The AUCs of the concentration-response curves are a summary of pharmacodynamic parameters for the purposes of predicting the clinical response such that the higher the AUC the lower the cytotoxic effect (efficacy or potency) of the drug. The regression hyperplane has been obtained using bi-dimensional smooth functions in a binary logistic GAM. When IDA is inactive at maximal IDA AUC, the AUC of CYT leads the prediction with a classical dose-response curve (curve on back, right-hand edge of plane). Conversely, When CYT is inactive IDA leads the prediction with a classical dose response curve (curve on back, left-hand edge of plane). When one of the drugs is very active (low AUC) the other shows a more limited effect, still consistent with higher AUC corresponding to higher probability of resistance. This behavior is coherent and consistent with the expectations; higher AUC of either drug (i.e. lower activity) implies higher probability of resistance. AUC: area under the curve, GAM: generalized additive model.

FIG. 13. Panel A: Empirical and smoothed (binormal) ROC curves of the probability of resistance obtained in the binary logistic GAM, wherein open circles are the pairs of sensitivity and 1-specificity values at the estimated discrete individual values of the probability of resistance (used as a marker to classify the patients as responder or resistant), the solid large circles represent the pairs of sensitivity and 1-specificity values at the selected cut-points that were obtained with each of the three criteria specified in the text: ‘MaxSpSe’ selects the point that maximizes both the sensitivity and the specificity; ‘Geometric’ selects the closest point to the (1,0) coordinate (left upper corner of the [sensitivity,1-specificity] plane); and ‘mMCT’ selects the point that minimizes a misclassification cost term that assigned a greater cost to false positives than to false negatives (prioritizes specificity over sensitivity. Panel B: Confusion matrix for MaxSpSe cutoff, wherein AUC: area under the curve, CI: confidence interval, FPF: false positive fraction, NPV: negative predictive value, PPV: positive predictive value, Se: sensitivity, Sp: specificity, TPF: true positive fraction.

FIG. 14. Kaplan-Meier plots of overall survival. The three panels (A, B and C) depict the pairs of survival functions for patients classified as responder (solid black lines) and resistant (solid red lines) according to the cut-points of the estimated probability of resistance that were obtained with each of the three criteria specified in the text (panel A: ‘MaxSpSe’, panel B: ‘Geometric’, and panel C: ‘mMCT’). The dashed lines represent the survival functions of clinical responders (black lines) and resistant patients (grey lines). The hazard ratios of death were obtained from a Cox regression model that used the patients who were predicted to be responder as the reference category (patients predicted to be resistant over patients predicted to be responder). CI: confidence interval, HR: hazard ratio FIG. 15. Comparison between clinical correlation of cytogenetics (panels A and B) and PM Test (panels C and D), on a cohort of 111 patients sharing both results. ROC curves (panels A and C) and confusion matrices for MaxSpSe cutoff (panels B and D). Deviance explained is 29.4% for cytogenetics, and 40.9% for PM test.

FIG. 16. A 3×3 drug combination matrix for a combination of drugs A and B, in which the number of informative datapoints is maximized, hence maximizing the accuracy in the estimation of the synergy parameter from interaction surface modelling. The calculation of these datapoints is based on intersections between arcs from the 10th, 50th and 90th percentiles of each EC₅₀ drug population value, and 3 lines splitting the quarter circle into 3 sectors of the same area.

FIG. 17. AUCxy value normalized as a reference to a benchmark area that is defined by a rectangle described by the same limits mentioned above that are defined by the whole population results and the 10%-90% response interval.

FIG. 18. Classifying of multiple treatments for a subject diagnosed with acute myeloid leukaemia based on the normalized sensitivity score for each treatment, according to the method for classifying the utility of drug combinations comprising a drug A and a drug B in treatment of a subject diagnosed with a disease. Treatments which said acute myeloid leukaemia is determined to be sensitive to (panel A) fall into the categories of more efficient and upper intermediate, while treatments which said acute myeloid leukaemia is determined to be resistant to (panel B) fall into the categories of lower intermediate and less efficient.

FIG. 19. Ranking of multiple treatments based on the normalized sensitivity score for each treatment, according to an alternative embodiment of the method for classifying the utility of drug combinations comprising a drug A and a drug B in treatment of a subject diagnosed with a disease. Sensitivity levels are shows in order from top best to bottom worst, colour coded green-sensitive (shown as pale grey), orange-indeterminate, red-resistant (shown as dark grey). Four patients with different degrees of sensitivity are shown, namely sensitive and standard patients (panel A) and resistant and very resistant patients (panel B).

FIG. 20. Validation of PM Test score vs clinical responses for one single CYT+IDA treatment in 112 first-line AML samples, where Res.=number of patients resistant to therapy, Sens.=number of patients sensitive to therapy and % Sens.=percentage of patients sensitive to therapy (panel A). Comparison of personalised medicine prediction test vs correlation test (panel B).

FIG. 21. Boxplots representing the distribution of EC₅₀ values provided by the population of samples stored in the database together with the 95% confidence interval associated with the estimation of the central median value. Vertical grey lines represent the results shown by the patient sample VIVIA-PMMM010431. Highlighted within boxes are the drugs used in the combination therapy; arrows indicate the EC₅₀ value for those drugs.

FIG. 22. Boxplots representing the distribution of EC₅₀ values provided by the population of samples stored in the database together with the 95% confidence interval associated with the estimation of the central median value. Vertical grey lines represent the results shown by the patient sample VIVIA-PMMM010431. Highlighted within boxes are the drugs used in the combination therapy; arrows indicate the EC₅₀ value for those drugs. The group boxed within the dotted line identifies the highly sensitive results (below 10th percentile) from the same drug family (mTor inhibitors) which may be considered as an alternative treatment for this patient.

FIG. 23. Boxplots representing the distribution of EC₅₀ values provided by the population of samples stored in the database together with the 95% confidence interval associated with the estimation of the central median value. Vertical grey lines represent the results shown by the patient sample VIVIA-PMMM130061. Highlighted within continuous-line boxes are the drugs used in the different therapies; arrows indicate the EC₅₀ value for those drugs. Highlighted within dotted-line boxes are alternative drugs with intermediate or high activity compared with population results.

FIG. 24. Boxplots representing the distribution of EC₅₀ values provided by the population of samples stored in the database together with the 95% confidence interval associated with the estimation of the central median value. Vertical grey lines represent the results shown by the patient sample VIVIA-PMALL07002. Highlighted within continuous-line boxes are the drugs used in the combined therapy; arrows indicate the EC₅₀ value for those drugs.

FIG. 25. Boxplots representing the distribution of EC₅₀ values provided by the population of samples stored in the database together with the 95% confidence interval associated with the estimation of the central median value. Vertical grey lines represent the results shown by the patient sample VIVIA-PMALL04001. Highlighted within continuous-line boxes are the drugs used in the combined therapy except Prednisolone that could not be tested; arrows indicate the EC₅₀ value for those drugs.

FIG. 26. Boxplots representing the distribution of EC₅₀ values provided by the population of samples stored in the database together with the 95% confidence interval associated with the estimation of the central median value. Vertical grey lines represent the results shown by the patient sample VIVIA-PMALL09001. Highlighted within continuous-line boxes are the drugs used in the combined therapy except Imatinib that could not be tested; arrows indicate the EC₅₀ value for those drugs.

### DETAILED DESCRIPTION OF THE INVENTION

The present invention discloses a method for determining the efficacy of treatment with a combination of drugs comprising a drug A and a drug B in a subject diagnosed with a disease. Analogously, the present invention discloses a system for determining the efficacy of treatment with a combination of drugs comprising a drug A and a drug B in a subject diagnosed with a disease.

The present invention also discloses a method for classifying the utility of drug combinations each comprising a drug A and a drug B in treatment of a subject diagnosed with a disease. Analogously, the present invention discloses a system for classifying the utility of drug combinations each comprising a drug A and a drug B in a subject diagnosed with a disease.

Said method and system for determining the efficacy of treatment with a combination of drugs comprising a drug A and a drug B in a subject diagnosed with a disease each comprise evaluating whether or not said combination of drugs will be effective in the treatment of said disease in said subject by determining whether said disease is sensitive to treatment with said combination of drugs in said subject or whether said disease is resistant to treatment with said combination of drugs in said subject. Said method and system for determining the efficacy of treatment therefore provide an evaluation of whether a given drug combination will be effective (or not) against said disease in said subject.

In contrast, said method and system for classifying the utility of drug combinations in treatment of a subject diagnosed with a disease each comprise scoring each of a number of combinations of drugs based on their utility in treatment of said disease in said subject, and classifying each combination of drugs based on said score. Said method and system for classifying the utility of drug combinations in treatment of a subject diagnosed with a disease therefore provide a therapeutic guide on the most suitable drug combination for treating said disease in said subject.

In the present invention, said combination of drugs comprises at least a drug A and a drug B. Preferably, said combination of drugs comprises at most three drugs. Thus, in one embodiment of the present invention, said combination of drugs also comprises a drug C. Said drug A, said drug B and said drug C are different.

Preferably, said drug A is selected from the group of idarubicin (IDA), cytarabine (CYT or ARA-C), fludarabine (FLU), etoposide (ETO), thioguanine (TIO), clofarabine (CLO), cladribine (CLA), daunorubicin (DNR or DAU), mitoxantrone (MIT), amsacrine (AMS), decitabine, doxorubicin, vincristine, cyclophosphamide, arsenic trioxide, 5-azacytidine, bortezomib, bendamustine, prednisolone, dexamethasone, thalidomide, L-asparaginase, imatinib, melphalan, maphosphamide, cisplatin, carmustine, tanespimycin, rapamycin, everolimus, temsirolimus, panobinostat, vorinostat, tipifarnib, perifosine, pomalidomide, lenalidomide, methylprednisolone, hydrocortisone, methotrexate and dasatinib. Said drug B is subsequently selected from the remaining group of drugs after drug A has been selected therefrom. Said drug C is subsequently selected from the remaining group of drugs after drugs A and B have been selected therefrom. In the method and system for determining the efficacy of treatment with a combination of drugs comprising a drug A and a drug B in a subject diagnosed with a disease, said drug A is preferably selected from the group of idarubicin and cytarabine and said drug B is that remaining after said drug A has been selected therefrom (i.e. drug A is idarubicin and drug B is cytarabine, or vice versa). In a preferred embodiment, drug A is idarubicin and drug B is cytarabine, or vice versa, and said combination of drugs comprises drug C selected from the group of fludarabine, etoposide, thioguanine and clofarabine.

In the present invention, said subject diagnosed with a disease is a subject diagnosed with a cancer of the hematopoietic and lymphoid tissues, preferably a leukemia, myeloma or lymphoma (meaning that the efficacy referred to in the method for determining the efficacy of treatment is antileukemia, antimyeloma or antilymphoma efficacy, respectively), more preferably a haematological cancer and even more preferably a leukemia or myeloma, yet more preferably acute myeloid leukemia (AML), multiple myeloma (MM) or acute lymphoid leukemia (ALL), most preferably acute myeloid leukemia.

In a preferred embodiment, when said disease is acute myeloid leukemia, drug A is selected from the group of idarubicin, cytarabine, fludarabine, etoposide, thioguanine, clofarabine, cladribine, daunorubicin, mitoxantrone and amsacrine, and drug B is subsequently selected from the remaining group of drugs after drug A has been selected therefrom. More preferably, when said disease is acute myeloid leukemia, drug A is idarubicin, fludarabine, daunorubicin or mitoxantrone and drug B is cytarabine, or vice versa, or drug A is idarubicin, daunorubicin, mitoxantrone or amsacrine and drug B is fludarabine, or vice versa, or drug A is idarubicin, daunorubicin, mitoxantrone or amsacrine and drug B is etoposide, or vice versa. Optionally, the combination of drugs comprises drug C selected from the remaining group of drugs listed under drug A in the aforementioned preferred embodiment after drugs A and B have been selected therefrom.

In another preferred embodiment, when said disease is multiple myeloma, drug A is selected from the group of bortezomib, bendamustine, prednisolone, dexamethasone and thalidomide, and drug B is subsequently selected from the remaining group of drugs after drug A has been selected therefrom, optionally followed by selection of drug C from the remaining group of drugs after drugs A and B have been selected therefrom. More preferably, when said disease is multiple myeloma, drug A is bendamustine or prednisolone and drug B is bortezomib, or vice versa, or drug A is dexamethasone and drug B is bortezomib, or vice versa, optionally wherein drug C is bendamustine or and thalidomide.

In yet another preferred embodiment, when said disease is acute lymphoid leukemia, drug A is selected from the group of prednisolone, idarubicin, cytarabine, fludarabine, daunorubicin, vincristine, cyclophosphamide, L-asparaginase and imatinib, and drug B is subsequently selected from the remaining group of drugs after drug A has been selected therefrom, optionally followed by selection of drug C from the remaining group of drugs after drugs A and B have been selected therefrom. More preferably, when said disease is acute lymphoid leukemia, drug A is daunorubicin or vincristine, drug B is prednisolone, or vice versa, and drug C is the drug remaining after drugs A and B have been selected therefrom or it is cyclophosphamide, L-asparaginase or imatinib (even more preferably when used in treatment of acute lymphoid leukemia, drug A is daunorubicin or vincristine, drug B is prednisolone, or vice versa, drug C is the drug remaining after drugs A and B have been selected therefrom, and said drugs A, B and C are administered in combination with cyclophosphamide and L-asparaginase or with imatinib). Alternatively, drug A is idarubicin, cytarabine or fludarabine and drug B is prednisolone, or vice versa, and drug C is selected from the remaining group of drugs after drugs A and B have been selected therefrom (even more preferably when used in treatment of acute lymphoid leukemia, drug A is idarubicin, cytarabine or fludarabine and drug B is prednisolone, or vice versa, drug C is selected from the remaining group of drugs after drugs A and B have been selected therefrom, and said drugs A, B and C are administered in combination with the remaining drug after drugs A, B and C have been selected therefrom.

Said subject may be a subject who is an adult subject (i.e. who has reached sexual maturity) diagnosed with said disease, preferably an adult subject who has not previously undergone first line or second-line treatment, an adult subject who has previously undergone first line treatment or an adult subject who has previously undergone second-line treatment.

In the present description, any given step referred to in any given method described herein, discloses steps that are carried out by the means comprised in the corresponding system described herein having the corresponding reference (e.g. step (a) in both methods of the present invention is a step that is carried out by the means (a) in both systems of the present invention). Thus, where the present description refers to a step having a particular reference in one or both of the methods described herein, it can also be understood that said step may be carried out by means having the same reference referred to in one or both of the systems described herein.

Both of said methods of the present invention comprise a step referred to under (a) of separating a tissue sample obtained from said subject into sub-samples. Said tissue sample is a blood, lymph or bone-marrow sample. Preferably, said tissue sample is a bone-marrow sample. Indeed, in one such embodiment of the present invention, said tissue sample comprises bone-marrow cells which are collected before the patient has been subjected to chemotherapy and/or radiation therapy. In an even more preferred embodiment, said bone marrow cells have a viability greater than or equal to 60% when incubated for 48 hours in the absence of drug A and/or drug B and/or drug C; and/or the bone-marrow cells were not present in a clot when obtained from said subject.

Separation of said sample into sub-samples involves dividing said sub-sample into portions. In one embodiment of each of the systems of the present invention, the means for carrying out the step of separating a tissue sample comprises a microfluidic stem cell separation device.

Both of said methods of the present invention also comprise a step (b) of carrying out the steps of:

(i) incubating a sub-sample for a time T of between 2 and 168 hours in the presence of said drug A at a concentration X; and

(ii) repeating step (b)(i) an additional (N−1) times, each time with a different sub-sample using a value for X that is different from that used in previous repetitions of step (b)(i);

wherein N is a whole number selected from between 5 and 10, inclusive;

and

(iii) incubating a sub-sample for said time T in the presence of said drug B at a concentration Y; and

(iv) repeating step (b)(iii) an additional (M−1) times, each time with a different sub-sample using a value for Y that is different from that used in previous repetitions of step (b)(iii); wherein M is a whole number selected from between 5 and 10, inclusive;

and

(v) incubating a sub-sample for said time Tin the presence of a combination of drugs comprising said drug A and said drug B, wherein
the concentration of said drug A is a concentration X corresponding to the concentration at the percentile value P_{Hα,A} from the distribution of X_{50,A} values obtained in said population of subjects each diagnosed with said disease, wherein percentile value P_{Hα,A} is calculated by the formula (A):

P_{Hα,A}=cos(α°)×H (A)
wherein:
   H corresponds to a reference percentile selected from the group of 10,

$$[ 10 + \frac{80}{R - 1} \times ( r - 1 ) ]$$

and 90, wherein:
   r is a whole number selected from between 2 and (R−1), inclusive
α is in degrees and is calculated from the formula:

$$α = \frac{90}{( W + 1 )} \times w$$

wherein:
   w is a whole number selected from between 1 and W, inclusive
the concentration of said drug B is a concentration Y corresponding to the concentration at the percentile value P_{Hα,B} from the distribution of Y_{50,B} values obtained in said population of subjects each diagnosed with said disease, wherein percentile value P_{Hα,B} is calculated by the formula (B):

P_{Hα,B}=cos(90°−α°)×H (B)

(vi) repeating step (b)(v) an additional (R−1) times, each time with a different sub-sample using a value for H that is different from that used in previous repetitions of step (b)(v), and using the same value for a that is used in step (b)(v); and

(vii) repeating steps (b)(v) and (b)(vi) an additional (W−1) times, each time with a different sub-sample using a value for a that is different from that used in previous repetitions of steps (b)(v) and (b)(vi);
wherein;
   R is a whole number selected from between 3 and 10, inclusive;
   W is a whole number selected from between 3 and 10, inclusive,
and wherein:
   X_{50,A} is the concentration of drug A exerting half of the maximum activity in a subject, estimated according to step (e)(i), below;
   Y_{50,B} is the concentration of drug B exerting half of the maximum activity in a subject, estimated according to step (e)(i), below;
and
(viii) incubating a sub-sample for said time T.

In one embodiment of each of the methods of the present invention, step (b) further comprises:

(ix) incubating a sub-sample for said time T in the presence of said drug C at a concentration Z; and

(x) repeating step (b)(ix) an additional (L−1) times, each time with a different sub-sample using a value for Z that is different from that used in previous repetitions of step (b)(ix); wherein Lisa whole number selected from between 5 and 10, inclusive;

and

(xi) incubating a sub-sample for said time T in the presence of a combination of drugs comprising said drug A and said drug C, wherein
the concentration of said drug A is a concentration X corresponding to the concentration at the percentile value P_{H′α′,A} from the distribution of X_{50,A} values obtained in said population of subjects each diagnosed with said disease, wherein percentile value P_{H′α′,A} is calculated by the formula (C):

P_{H′α′,A}=cos(α′°)×H′ (C)
wherein:
   H′ corresponds to a reference percentile selected
   from the group of 10,

$$[ 10 + \frac{80}{{R}^{′} - 1} \times ( {r}^{′} - 1 ) ]$$

and 90, wherein:
   r is a whole number selected from between 2 and (R′−1), inclusive
α′ is in degrees and is calculated from the formula:

$${α}^{′} = \frac{90}{( {W}^{′} + 1 )} \times {w}^{′}$$

wherein:
   w′ is a whole number selected from between 1 and W, inclusive
the concentration of said drug C is a concentration Z corresponding to the concentration at the percentile value P_{H′α′,C} from the distribution of Z_{50,C} values obtained in said population of subjects each diagnosed with said disease, wherein percentile value P_{H′α′,C} is calculated by the formula (D):

P_{H′α′,C}=cos(90°−α′°)×H′ (D)

(xii) repeating step (b)(xi) an additional (R′−1) times, each time with a different sub-sample using a value for H′ that is different from that used in previous repetitions of step (b)(xi), and using the same value for a′ that is used in step (b)(xi); and

(xiii) repeating steps (b)(xi) and (b)(xii) an additional (W′−1) times, each time with a different sub-sample using a value for a′ that is different from that used in previous repetitions of steps (b)(xi) and (b)(xii);
wherein;
   R′ is a whole number selected from between 3 and 10, inclusive;
   W is a whole number selected from between 3 and 10, inclusive, and;

(xiv) incubating a sub-sample for said time T in the presence of a combination of drugs comprising said drug B and said drug C, wherein
the concentration of said drug B is a concentration Y corresponding to the concentration at the percentile value P_{H″α″,B} from the distribution of Y_{50,B} values obtained in said population of subjects each diagnosed with said disease, wherein percentile value P_{H″α″,B} is calculated by the formula (E):

P_{H″α″,B}=cos(α″°)×H″ (E)
wherein:
   H″ corresponds to a reference percentile selected
   from the group of 10,

$$[ 10 + \frac{80}{{R}^{″} - 1} \times ( {r}^{″} - 1 ) ]$$

and 90, wherein:
   R″ is a whole number selected from between 2 and (R″−1), inclusive;
α″ is in degrees and is calculated from the formula:

$${α}^{″} = \frac{90}{( {W}^{″} + 1 )} \times {w}^{″}$$

wherein:
   w″ is a whole number selected from between 1 and W″, inclusive
the concentration of said drug C is a concentration Z corresponding to the obtained in said population of subjects each diagnosed with said disease, wherein percentile value P_{H″α″,C} is calculated by the formula (F):

P_{H″α″,C}=cos(90°−α″)×H″ (F)

(xv) repeating step (b)(xiv) an additional (R″−1) times, each time with a different sub-sample using a value for H″ that is different from that used in previous repetitions of step (b)(xiv), and using the same value for α″ that is used in step (b)(xiv); and

(xvi) repeating steps (b)(xiv) and (b)(xv) an additional (W′−1) times, each time with a different sub-sample using a value for α″ that is different from that used in previous repetitions of steps (b)(xiv) and (b)(xv);
wherein;
   R″ is a whole number selected from between 3 and 10, inclusive;
   W′ is a whole number selected from between 3 and 10, inclusive,

and wherein:
X_{50,A} is the concentration of drug A exerting half of the maximum activity in a subject, estimated according to step (e)(i);
Y_{50,B} is the concentration of drug B exerting half of the maximum activity in a subject, estimated according to step (e)(i);
Z_{50,C} is the concentration of drug C exerting half of the maximum activity in a subject, estimated according to step (e)(i), below.

The X_{50,A}, Y_{50,B} and Z_{50,C} values obtained in a population of subjects each diagnosed with said disease are preferably obtained from a population of at least 5 of said subjects, even more preferably at least subjects, still more preferably at least 20 subjects, most preferably at least 30 subjects. Each of said values is obtained for each subject in said population of subjects.

Population models are built from scratch using the aforementioned minimum number of samples or when the number of samples accumulated for a model (NT) is more than a defined threshold number (Na). Preferably Nu is a number that corresponds with 10% to 20% of the samples used to build the previous model, and more preferably Nu is a number that corresponds with 20% of the samples used to build the previous model. A valid model should have an Objective Function Value (OFV) at least 4 points lower than the previous model but also have biological sensible model parameters and meet several conditions like Inter-Patient Variability (IPV), Standard Error (SE) and Quality Control check values. If not, then the model should be rebuilt again after 10 new samples. When the new model conditions are met then all the new model data is stored in the database and used until replaced by a subsequent model.

The steps for determining the respective concentrations X, Y and Z of drugs A, B and C used in steps (b)(i) and (b)(ii), (b)(iii) and (b)(iv), and (b)(ix) and (b)(x) of the present invention preferably first comprise selecting for each drug the end-point concentrations u1 and u2 of the U % trimmed range of the distribution of the concentrations at which said drug results in a reduction of more than V % of the number of live cells LCTi of each cell type identified according to step (c) which is obtained in said population of subjects, wherein U is between 1% and 50% and V is at least 1%, more preferably wherein U is between 2% and 30% and V is at least 5%, even more preferably wherein U is between 5% and 75% and V is at least 10%, still more preferably wherein the U % trimmed range is the interdecile range or interquartile range and V i′ at least 10%. Said concentrations are determined by:

(i) selecting a concentration from the group of: u1,

$$[ u 1 + \frac{( u 1 + u 2 )}{{N}^{′} - 1} \times ( {n}^{′} - 1 ) ]$$

and u2, wherein:
n′ is a whole number selected from between 2 and (N′−1), inclusive; and

(ii) repeating step (i) an additional (N′−1) times, each time with a concentration value that is different from that used in previous repetitions of step (i);

wherein N′ is N, M or P, depending on whether said drug is drug A, drug B or drug C, respectively;

The steps for determining the respective concentrations X, Y and Z of drugs A, B and C used in steps (b)(v) to (b)(vii), (b)(xi) to (b)(xiii) and (b)(xiv) to (b)(xvi) of the present invention comprise the steps of a method for improving the accuracy in the estimation of synergism of a combination of drugs. Said method is a statistical method of providing a distribution of the concentrations of any two drugs in combination experiments in which the number of informative datapoints is maximized, and maximizing the accuracy in the estimation of the synergy parameter from interaction surface modelling. Theoretically, drugs interaction experiments require up to 50 data points of paired concentrations of each drug to cover properly enough area to fit well to a surface interaction model and allow an accurate estimation of synergism. Experimentally, this is a severe limitation as it would require an amount of sample that is never available. The method described herein minimizes the number of points to the most representative concentrations of drug at which each drug is active according to historical stored data from dose response curves from multiple patient samples (herein, EC₅₀ distributions of each drug).

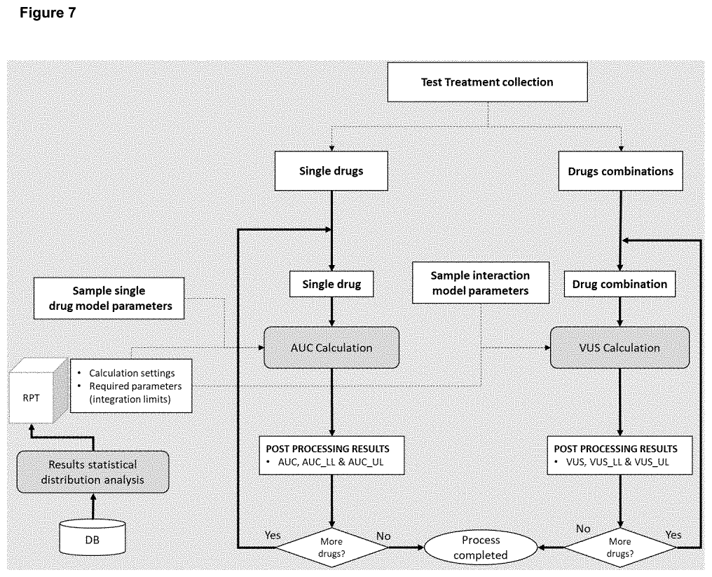

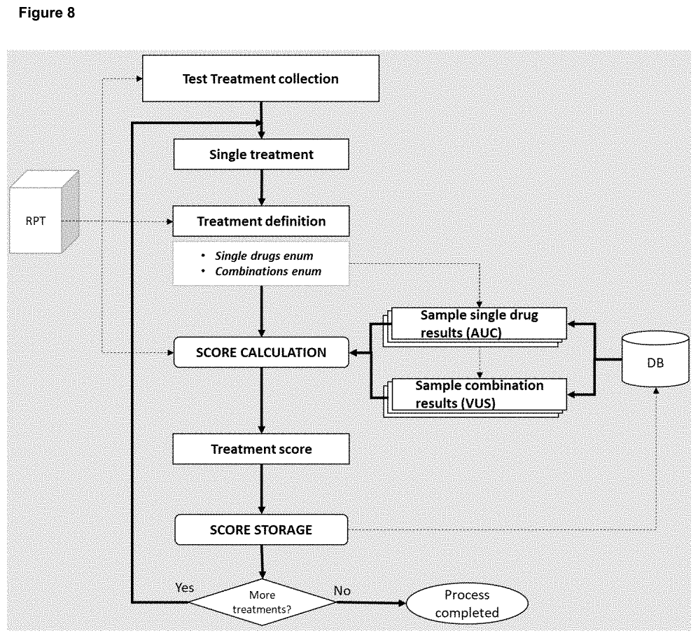

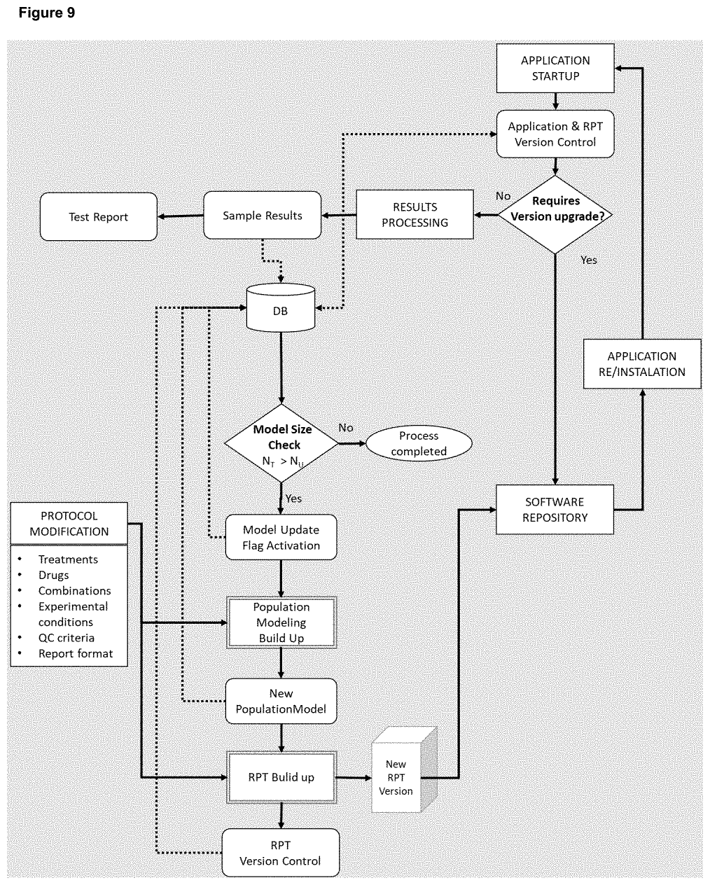

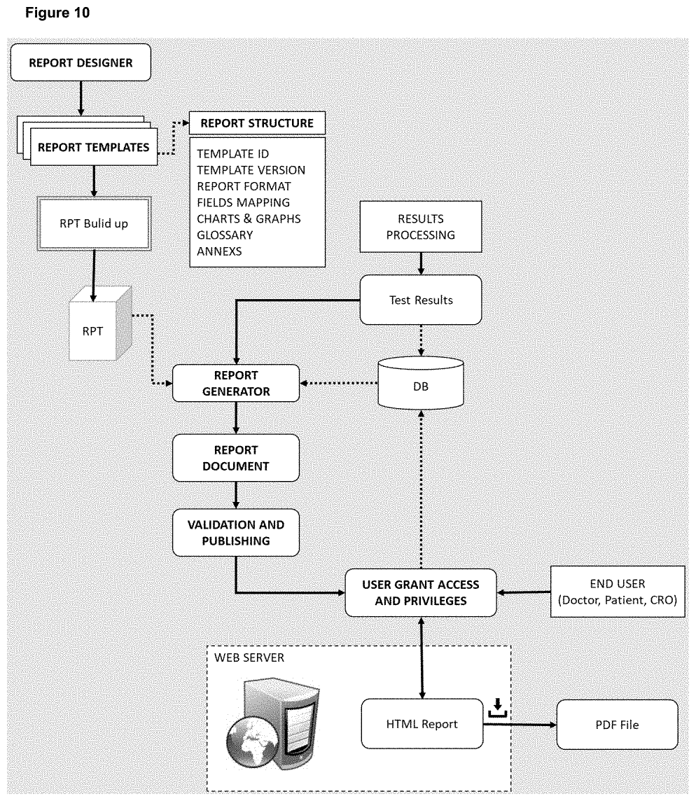

Dose response curves from multiple patient samples were analysed to determine the concentration ratios which most accurately model the interaction surface. In FIG. 7 these (R=3)×(W=3)=9 points correspond with the 10th, 50th and 90th percentiles of each EC₅₀ drug population values, whereby three quarter-circles are plotted using each drug percentile as a radius and origin as centre. In addition, three straight lines are plotted using origin as starting point and with a given angle in order to split the different circles into sectors of the same size angle. Finally, the intersections between lines and circles are calculated and converted into concentration units. The X values from each point belong to drug A and the Y values to drug B.

In a preferred embodiment, N=M and R=W, wherein N(=M) is selected from a whole number from 5 to 10, inclusive, and R (=W) is selected from a whole number from 3 to 10, inclusive. In a more preferred embodiment of the present invention, N=M and R=W, wherein N(=M) is selected from the group of whole numbers 8, 9 and 10, and R (=W) is selected from the group of whole numbers 3, 4 and 5, still more preferably N(=M)=8 and R (=W)=3. When R=W is 3, a is selected from the group of 22.5°, 45° and 67.5°. When said combination of drugs additionally comprises a drug C, preferably N=M=L and R=W=R′=W′=R″=W″, wherein N(=M=L) is selected from a whole number from 5 to 10, inclusive, and R (=W=R′=W′=R″=W″) is selected from a whole number from 3 to 10, inclusive. In an even more preferred embodiment of the present invention, N=M=L and R=W=R′=W′=R″=W″, wherein N(=M=L) is selected from the group of whole numbers 8, 9 and 10, and R (=W=R′=W′=R″=W″) is selected from the group of whole numbers 3, 4 and 5, still more preferably N(=M=L)=8 and R (=W=R′=W′=R″=W″)=3. When R=W=R′=W′=R″=W′ is 3, each of α, α′ and α″ is selected from the group of 22.5°, 45° and 67.5°.

Each of said steps of incubation involve incubation of a sub-sample which has not been subjected to prior incubation with a combination of drugs according to the methods of the present invention.

Preferably, the time T of incubation is a time of between 24 and 72 hours, more preferably a time of between 36 and 60 hours, and, in an even more preferred embodiment, 48 hours. In one embodiment of each of the systems of the present invention, the means for carrying out the steps of incubating comprises a cell culture incubator.

Both of said methods of the present invention also comprise a step (c) of adding at least one marker to each sub-sample incubated in step (b) to identify at least one cell-type (CTᵢ) therein. Each ith cell type identified differs depending on said marker added. In one embodiment of the methods of the present invention, step (c) comprises:
(i) adding at least one conjugated antibody to each sub-sample incubated in step (b) to identify at least one pathological cell-type therein; and
(ii) adding at least one cell death or apoptosis marker such as anexin, iodure propidium, 7-AAD, Draq5, Hoechst or DAPI to each sub-sample incubated in step (b) to identify apoptotic cells therein.

Preferably, said conjugated antibody identifies cancer cells, more preferably cancer cells of the hematopoietic and lymphoid tissues, even more preferably leukemia or lymphoma cells, still more preferably leukemia cells, and most preferably acute myeloid leukemia cells. By adding said at least one marker, preferably at least one conjugated antibody and at least one cell death or apoptosis marker, to each sub-sample incubated in step (b), living pathological cells of the disease are identified in preparation for step (d). In one embodiment of each of the systems of the present invention, the means for carrying out the step of adding at least one marker comprises a pipette or an injector such as a syringe or dispenser.

Thus, both of said methods of the present invention also comprise a step (d) of counting the number of live cells (LCTi) of each cell-type identified in step (c) which remain after incubation of each sub-sample according to step (b). In the aforementioned embodiment wherein at least one conjugated antibody and at least one cell death or apoptosis marker are added as markers in step (c), step (d) comprises counting the number of live cells (LCTi) remaining after incubation of each sub-sample by counting the number of cells of said at least one pathological cell-type identified according to step (c)(i) which are not identified as apoptotic according to step (c)(ii). In another embodiment of each of the systems of the present invention, the means for carrying out the steps of counting the number of live pathological cells comprises a cytometer, preferably a flow cytometer, an image cytometer or a cytophotometer, more preferably a flow cytometer.

Both of said methods of the present invention also comprise a step (e) of determining for each cell-type identified in step (c):

(i) pharmacodynamic parameter values;

(ii) activity marker values;

and

(iii) normalized marker values. Thus, step (e) comprises determining for each cell-type (CT) identified in step (c), at least one of each of said values, wherein:
pharmacodynamic parameter values comprise at least one pharmacodynamic parameter value for drug A and/or at least one pharmacodynamic parameter value for drug B, wherein:
   each pharmacodynamic parameter value for drug A is estimated from a single drug dose-response pharmacodynamic mixed effects non-linear population model by fitting a formula to experimental values of LCTi counted according to step (d) after incubating sub-samples for each subject in the population according to steps (b)(i) and (ii); and
   each pharmacodynamic parameter value for drug B is estimated from a single drug dose-response pharmacodynamic mixed effects non-linear population model by fitting a formula to experimental values of LCTi counted according to step (d) after incubating sub-samples for each subject in the population according to steps (b)(iii) and (iv), wherein said population comprises said subject and other subjects diagnosed with said disease;
activity marker values comprise at least one activity marker value for drug A, at least one activity marker value for drug B and/or at least one activity marker value for drugs A and B,
wherein:
   each activity marker value for drug A is calculated from said pharmacodynamic parameter value or values for drug A estimated in step (e)(i),
   each activity marker value for drug B is calculated from said pharmacodynamic parameter value or values for drug B estimated in step (e)(i),
   each activity marker value for drugs A and B is calculated from a specific model made by fitting a formula to said pharmacodynamic parameter value or values for drug A and said pharmacodynamic parameter value or values for drug B which are estimated in step (e)(i), as well as to experimental values of LCTi counted according to step (d) after incubating sub-samples for each subject in the population according to steps (b)(v) to (vii); and
normalized marker values comprise at least one normalized marker value for drug A, at least one normalized marker value for drug B and/or at least one normalized marker value for drugs A and B, wherein:
   each normalized marker value for drug A is calculated from the ratio of each activity marker value for drug A that is calculated in step (e)(ii) relative to a corresponding value from the distribution of said activity marker value for said population;
   each normalized marker value for drug B is calculated from the ratio of each activity marker value for drug B that is calculated in step (e)(ii) relative to a corresponding value from the distribution of said activity marker value for drug B for said population;
   each normalized marker value for drugs A and B is calculated from the ratio of each activity marker value for drugs A and B that is calculated in step (e)(ii) relative to a corresponding value from the distribution of said activity marker value for drugs A and B for said population.

In one embodiment of the methods of the present invention, when said drug combination comprises a drug A and a drug B and a drug C,
the pharmacodynamic parameter values determined in step (e)(i) optionally additionally comprise at least one pharmacodynamic parameter value for drug C, wherein:
   each pharmacodynamic parameter value for drug C is estimated from a single drug dose-response pharmacodynamic mixed effects non-linear population model by fitting a formula to experimental values of LCTi counted according to step (d) after incubating sub-samples for each subject in the population according to steps (b)(ix) and (x); and the activity marker values determined in step (e)(ii) optionally additionally comprise at least one activity marker value for drug C, at least one activity marker value for drugs A and C, and/or at least one activity marker value for drugs B and C, wherein:
each activity marker value for drug C is calculated from said pharmacodynamic parameter value or values for drug C estimated in step (e)(i),
   each activity marker value for drugs A and C is calculated from a specific model made by fitting a formula to said pharmacodynamic parameter value or values for drug A and said pharmacodynamic parameter value or values for drug C which are estimated in step (e)(i), as well as to experimental values of LCTi counted according to step (d) after incubating sub-samples for each subject in the population according to steps (b)(xi) to (xiii);
   each activity marker value for drugs B and C is calculated from a specific model made by fitting a formula to said pharmacodynamic parameter value or values for drug B and said pharmacodynamic parameter value or values for drug C which are estimated in step (e)(i), as well as to experimental values of LCTi counted according to step (d) after incubating sub-samples for each subject in the population according to steps (b)(xiv) to (xvi);
the normalized marker values determined in step (e)(iii) optionally additionally comprise normalized marker values comprising at least one normalized marker value for drug C, at least one normalized marker value for drugs A and C, and/or at least one normalized marker value for drugs B and C, wherein:
   each normalized marker value for drug C is calculated from the ratio of each activity marker value for drug C that is calculated in step (e)(ii) relative to a corresponding value from the distribution of said activity marker value for said population;
   each normalized marker value for drugs A and C is calculated from the ratio of each activity marker value for drugs A and C that is calculated in step (e)(ii) relative to a corresponding value from the distribution of said activity marker value for drugs A and C for said population;
   each normalized marker value for drugs B and C is calculated from the ratio of each activity marker value for drugs B and C that is calculated in step (e)(ii) relative to a corresponding value from the distribution of said activity marker value for drugs B and C for said population.

In an embodiment of each of said methods of the present invention, the pharmacodynamic parameter values determined in step (e)(i) comprise an X_{50,A} value, a LCTi_{0,A} value, an E_{max,A} value, a γ_{A} value, a Y_{50,B} value, an LCTi_{0,B} value, an E_{max,B} value and/or a γ_{B} value, wherein:
said X_{50,A}, LCTi_{0,A}, E_{max,A} and γ_{A} values are estimated from a single drug dose-response pharmacodynamic mixed effects non-linear population model determined by fitting the formula (I) to experimental values of LCTi counted according to step (d) after incubating sub-samples for each subject in a population according to steps (b)(i) and (b)(ii) obtained for each concentration X of drug A:

$$L C T i = {LCTi}_{0 , A} \times [ 1 - {E}_{max , A} \times \frac{{X}^{{γ}_{A}}}{{X}^{{γ}_{A}} + {X}_{50 , A}^{{γ}_{A}}} ] \qquad ( I )$$

said Y_{50,B}, LCTi_{0,B}, E_{max,B} and γ_{B} values are estimated from a single drug dose-response pharmacodynamic mixed effects non-linear population model determined by fitting the formula (II) to experimental values of LCTi counted according to step (d) after incubating sub-samples for each subject in said population according to steps (b)(iii) and (b)(iv) obtained for each concentration Y of drug B:

$$L C T i = {LCTi}_{0 , B} \times [ 1 - {E}_{max , B} \times \frac{{Y}^{{γ}_{B}}}{{Y}^{{γ}_{B}} + {Y}_{50 , B}^{{γ}_{B}}} ] \qquad ( II )$$

wherein said population comprises said subject and other subjects diagnosed with said disease;
wherein:
   X=concentration of drug A;
   X_{50,A} is the concentration of drug A exerting half of maximum activity;
   LCTi_{0,A} is the basal (pre-incubation) number of LCTi and is equal to the LCTi counted after incubating a sub-sample in the absence of a drug according to the step referred to in (b)(viii);
   E_{max,A}, is the maximum fractional decrease of LCTi_{0,A} caused by drug A;
   γ_{A} is the steepness of the LCTi vs concentration curve for drug A;
   Y=concentration of drug B;
   Y_{50,B} is the concentration of drug B exerting half of maximum activity;
   LCTi_{0,B} is the basal (pre-incubation) number of LCTi and is equal to the LCTi counted after incubating a sub-sample in the absence of a drug according to the step referred to in (b)(viii);
   E_{max,B}, is the maximum fractional decrease of LCTi_{0,B} caused by drug B;
   γ_{B} is the steepness of the LCTi vs concentration curve for drug B.

In a more preferred embodiment of each of said methods of the present invention, when said combination of drugs additionally comprises drug C, the pharmacodynamic parameter values determined in step (e)(i) optionally further comprise a Z_{50,C} value, a LCTi_{0,C} value, an E_{max,C} value and/or a γ_{C} value, wherein:
said Z_{50,C}, LCTi_{0,C}, E_{max,C} and γ_{C} values are estimated from a single drug dose-response pharmacodynamic mixed effects non-linear population model determined by fitting the formula (XI) to experimental values of LCTi counted according to step (d) after incubating sub-samples for each subject in said population according to steps (b)(ix) and (b)(x) obtained for each concentration Z of drug C:

$$L C T i = {LCTi}_{0 , C} \times [ 1 - {E}_{max , C} \times \frac{{Z}^{{γ}_{C}}}{{Z}^{{γ}_{C}} + {Z}_{50 , C}^{{γ}_{C}}} ] \qquad ( XI )$$

wherein:
   Z=concentration of drug C;
   Z_{50,C} is the concentration of drug C exerting half of maximum activity;
   LCTi_{0,C} is the basal (pre-incubation) number of LCTi and is equal to the LCTi counted after incubating a sub-sample in the absence of a drug according to the step referred to in (b)(viii);
   E_{max,C} is the maximum fractional decrease of LCTi_{0,C} caused by drug C;
   γ_{C} is the steepness of the LCTi vs concentration curve for drug C.

In an embodiment of each of said methods of the present invention, the activity marker values determined in step (e)(ii) comprise an AUC_{xy,A} value, an AUC_{xy,B} value, an α_{AB} value and/or a VUS_{AB} value, wherein:
said AUC_{xy,A} value is calculated using the formula (III):

AUC_{xy,A}=AUC_{x,A}−A_{y:10-90,A} (III)
wherein:
said AUC_{x,A} value is the integral between two drug concentrations X′ and X″ of a function derived from formula (I) for the % survival after incubating sub-samples according to steps (b)(i) and (b)(ii) obtained for each concentration X of drug A wherein LCTi_{0,A} is considered as 100% survival, and is calculated using the formula (IV):

$$A U {C}_{x , A} = {\int }_{{X}^{′}}^{{X}^{″}} 100 \times [ 1 - {E}_{max , A} \times \frac{{X}^{{γ}_{A}}}{{X}^{{γ}_{A}} + {X}_{50 , A}^{{γ}_{A}}} ] d X \qquad ( IV )$$

wherein drug concentrations X′ and X″ correspond to the concentrations of the 20^{th} and 80^{th} percentiles of the X_{50,A} values obtained in said population of subjects each diagnosed with said disease, wherein X_{50,A} was calculated for each subject in said population according to steps (a) to (e)(i);
and
said A_{y:10-90,A} value is the surface from AUC_{x,A} that falls outside the 10% and 90% boundaries of the % survival, wherein LCTi_{0,A} is considered as 100% survival;
and
said AUC_{xy,B} value is calculated using the formula (V):

AUC_{xy,B}=AUC_{x,B}−A_{y:10-90,B} (V)
wherein:
said AUC_{x,B} value is the integral between two drug concentrations Y′ and Y″ of a function derived from formula (II) for the % survival after incubating sub-samples according to steps (b)(iii) to (b)(iv) obtained for each concentration Y of drug B, wherein LCTi_{0,B} is considered as 100% survival, and is calculated using the formula (VI):

$$A U {C}_{x , B} = {\int }_{{Y}^{′}}^{{Y}^{″}} 100 \times [ 1 - {E}_{max , B} \times \frac{{Y}^{{γ}_{B}}}{{Y}^{{γ}_{B}} + {Y}_{50 , B}^{{γ}_{B}}} ] d Y \qquad ( VI )$$

wherein drug concentrations Y′ and Y″ correspond to the concentrations of the 20th and 80th percentiles of the Y_{50,B} values obtained in said population of subjects each diagnosed with said disease, wherein Y_{50,B} was calculated for each subject in said population according to steps (a) to (e)(i);
and
said A_{y:10-90,B} value is the surface from AUC_{x,B} that falls outside the 10% and 90% boundaries of the % survival, wherein LCTi_{0,A} is considered as 100% survival;
and
said VUS_{AB} value is calculated using the formula (VII), wherein said VUS_{AB} value is the double integral between two drug concentrations X′ and X″ for drug A and two drug concentrations Y′ and Y″ for drug B of the model function of the natural log of LCTi counted after incubating sub-samples according to steps (b)(v) to (b)(vii), wherein LCTi_{0,A}=LCTi_{0,B} and is considered as 100% survival, and is calculated using the formula (VII),

$${VUS}_{AB} = {\int }_{{X}^{′}}^{{X}^{″}} {\int }_{{Y}^{′}}^{{Y}^{″}} 100 \times [ 1 - \frac{\begin{matrix}( {E}_{mαx , A} \times \frac{X}{X + {X}_{50 , A}} + {E}_{mαx , B} \times \\ \frac{Y}{Y + {Y}_{50 , B}} + {α}_{A B} \times {E}_{mαx , A} \times {E}_{mαx , B} \times \\ {\frac{X}{X + {X}_{50 , A}} \times \frac{Y}{Y + {Y}_{50 , B}} )}^{{γ}_{int , AB}}\end{matrix}}{1 + {( \frac{X}{{X}_{50 , A}} + \frac{Y}{{Y}_{50 , B}} + {α}_{A B} \times \frac{X}{{X}_{50 , A}} \times \frac{Y}{{Y}_{50 , B}} )}^{{γ}_{int , AB}}} ] dXdY \qquad ( VII )$$

wherein:
   drug concentrations X′ and X″ correspond to the concentrations of the 20^{th} and 80^{th} percentiles of the X_{50,A} values obtained in said population of subjects each diagnosed with said disease, wherein X_{50,A} was calculated for each subject in said population according to steps (a) to (e)(i);
   drug concentrations Y′ and Y″ correspond to the concentrations of the 20th and 80th percentiles of the Y_{50,B} values obtained in said population of subjects each diagnosed with said disease, wherein Y_{50,B} was calculated for each subject in said population according to steps (a) to (e)(i);
   E_{max,A}=maximum fractional decrease in LPC caused by drug A;
   E_{max,B}=maximum fractional decrease in LPC caused by drug B;
   X_{50,A}=EC₅₀ concentration of drug A exerting half of E_{max,A};
   Y_{50,B}=EC₅₀ concentration of drug B exerting half of E_{max,B};
   X=concentration of drug A;
   Y=concentration of drug B;

$${y}_{int , AB} = {γ}_{{A}^{\times }} \frac{\frac{X}{{X}_{50 , A}}}{\frac{X}{{X}_{50 , A}} + \frac{Y}{{Y}_{50 , B}}} + ( 1 - \frac{\frac{X}{{X}_{50 , A}}}{\frac{X}{{X}_{50 , A}} + \frac{Y}{{Y}_{50 , B}}} ) \times {γ}_{B}$$

wherein:
   γA=steepness of the LCTi vs concentration curve for drug A;
   γB=steepness of the LCTi vs concentration curve for drug B; and
α_{AB}=synergy parameter estimated from a two-drug surface interaction model determined by fitting the formula (VII′) to experimental values of LCTi counted according to step (d) after incubating sub-samples for said subject according to the steps referred to in (b)(i) and (b)(ii) obtained for each concentration X of drug A (where Y=Z=0), the steps referred to in (b)(iii) and (b)(iv) obtained for each concentration Y of drug B (where X=Z=0), and the steps referred to in b(v), b(vi) and b(vii) obtained for each pair of concentrations of the combination of drug A and drug B:

$$L C T i = L C T {i}_{0 , AB} \times [ 1 - \frac{\begin{matrix}( {E}_{max , A} \times \frac{X}{X + {X}_{50 , A}} + {E}_{max , B} \times \\ \frac{Y}{Y + {Y}_{50 , B}} + {α}_{A B} \times {E}_{max , A} \times \\ {{E}_{max , B} \times \frac{X}{X + {X}_{50 , A}} \times \frac{Y}{Y + {Y}_{50 , B}} )}^{{γ}_{int , AB}}\end{matrix}}{1 + {( \frac{X}{{X}_{50 , A}} + \frac{Y}{{Y}_{50 , B}} + {α}_{A B} \times \frac{X}{{X}_{50 , A}} \times \frac{Y}{{Y}_{50 , B}} )}^{{γ}_{int , AB}}} ] . \qquad ( {VII}^{′} )$$

In a more preferred embodiment of each of said methods of the present invention, when said combination of drugs additionally comprises drug C, the activity marker values determined in step (e)(ii) optionally further comprise an AUC_{xy,C} value, an α_{AC} value, a VUS_{AC} value, an α_{BC} value and/or a VUS_{BC} value, wherein:
said AUC_{xy,C} value is calculated using the formula (XII):

AUC_{xy,C}=AUC_{x,C}−A_{y:10-90,C} (XII)
wherein:
said AUC_{x,C} value is the integral between two drug concentrations Z′ and Z″ of a function derived from formula (XI) for the % survival after incubating sub-samples according to steps (b)(ix) and (b)(x) obtained for each concentration Z of drug C wherein LCTi_{0,C} is considered as 100% survival, and is calculated using the formula (XIII):

$$A U {C}_{x , C} = {\int }_{{Z}^{′}}^{{Z}^{″}} 100 \times [ 1 - {E}_{max , C} \times \frac{{Z}^{{γ}_{C}}}{{Z}^{{γ}_{C}} + {Z}_{50 , C}^{{γ}_{C}}} ] d Z \qquad ( XIII )$$

wherein drug concentrations Z′ and Z″ correspond to the concentrations of the 20th and 80th percentiles of the Z_{50,C} values obtained in said population of subjects each diagnosed with said disease, wherein Z_{50,C} was calculated for each subject in said population according to steps (a) to (e)(i);
and
said A_{y:10-90,C} value is the surface from AUC_{x,C} that falls outside the 10% and 90% boundaries of the % survival, wherein LCTi_{0,C} is considered as 100% survival;
and
said VUS_{AC} value is calculated using the formula (XIV), wherein said VUS_{AC} value is the double integral between two drug concentrations X′ and X″ for drug A and two drug concentrations Z′ and Z″ for drug C of the model function of the natural log of LCTi counted after incubating sub-samples according to steps (b)(xi) to (b)(xiii), wherein LCTi_{0,A}=LCTi_{0,C} and is considered as 100% survival,

$$V U {S}_{A C} = {\int }_{{X}^{′}}^{{X}^{″}} {\int }_{{Y}^{′}}^{{Y}^{″}} 100 \times [ 1 - \frac{\begin{matrix}( {E}_{mαx , A} \times \frac{X}{X + {X}_{50 , A}} + {E}_{mαx , C} \times \\ \frac{Z}{Z + {Z}_{50 , C}} + {α}_{A C} \times {E}_{mαx , A} \times \\ {{E}_{m α x , C} \times \frac{X}{X + {X}_{50 , A}} \times \frac{Z}{Z + {Z}_{50 , C}} )}^{{γ}_{int , AC}}\end{matrix}}{\begin{matrix}□ \\ {( \frac{X}{{X}_{50 , A}} + \frac{Z}{{Z}_{50 , C}} + {α}_{AC} \times \frac{X}{{X}_{50 , A}} \times \frac{Z}{{Z}_{50 , C}} )}^{{γ}_{int , AC}}\end{matrix}} ] dXdZ \qquad ( XIV )$$

wherein:
   drug concentrations X′ and X″ correspond to the concentrations of the 20^{th} and 80^{th} percentiles of the X_{50,A} values obtained in said population of subjects each diagnosed with said disease, wherein X_{50,A} was calculated for each subject in said population according to steps (a) to (e)(i);
   drug concentrations Z′ and Z″ correspond to the concentrations of the 20^{th} and 80^{th} percentiles of the Z_{50,C} values obtained in said population of subjects each diagnosed with said disease, wherein Z_{50,C} was calculated for each subject in said population according to steps (a) to (e)(i);
   E_{max,A}=maximum fractional decrease in LPC caused by drug A;
   E_{max,C}=maximum fractional decrease in LPC caused by drug C;
   X_{50,A}=EC₅₀ concentration of drug A exerting half of E_{max,A};
   Z_{50,C}=EC₅₀ concentration of drug C exerting half of E_{max,C};
   X=concentration of drug A;
   Z=concentration of drug C;

$${y}_{int , AC} = {γ}_{A} \times \frac{\frac{X}{{X}_{50 , A}}}{\frac{X}{{X}_{50 , A}} + \frac{Z}{{Z}_{50 , C}}} + ( 1 - \frac{\frac{X}{{X}_{50 , A}}}{\frac{X}{{X}_{50 , A}} + \frac{Z}{{Z}_{50 , C}}} ) \times {γ}_{C}$$

wherein:
   γA=steepness of the LCTi vs concentration curve for drug A;
   γC=steepness of the LCTi vs concentration curve for drug C;
and
α_{AC}=synergy parameter estimated from a two-drug surface interaction model determined by fitting the formula (XIV′) to experimental values of LCTi counted according to step (d) after incubating sub-samples for said subject according to the steps referred to in (b)(i) and (b)(ii) obtained for each concentration X of drug A (where Y=Z=0), the steps referred to in (b)(ix) and (b)(x) obtained for each concentration Z of drug C (where X=Y=0), and the steps referred to in b(xi), b(xii) and b(xiii) obtained for each pair of concentrations of the combination of drug A and drug C:

$$L C T i = L C T {i}_{0 , AC} \times [ 1 - \frac{\begin{matrix}( {E}_{max , A} \times \frac{X}{X + {X}_{50 , A}} + {E}_{max , C} \times \\ \frac{Z}{Z + {Z}_{50 , C}} + {α}_{A C} \times {E}_{max , A} \times {E}_{max , C} \times \\ {\frac{X}{X + {X}_{50 , A}} \times \frac{Z}{Z + {Z}_{50 , C}} )}^{{γ}_{int , AC}}\end{matrix}}{1 + {( \frac{X}{{X}_{50 , A}} + \frac{Z}{{Z}_{50 , C}} + {α}_{AC} \times \frac{X}{{X}_{50 , A}} \times \frac{Z}{{Z}_{50 , C}} )}^{{γ}_{int , AC}}} ] ; \qquad ( {XIV}^{′} )$$

said VUS_{BC} value is calculated using the formula (XV), wherein said VUS_{BC} value is the double integral between two drug concentrations Y′ and Y″ for drug B and two drug concentrations Z′ and Z″ for drug C of the model function of the natural log of LCTi counted after incubating sub-samples according to steps (b)(xiv) to (b)(xvi), wherein LCTi_{0,B}=LCTi_{0,C} and is considered as 100% survival,

$${VUS}_{B C} = {\int }_{{X}^{′}}^{{X}^{″}} {\int }_{{Y}^{′}}^{{Y}^{″}} 100 \times [ 1 - \frac{\begin{matrix}( {E}_{mαx , B} \times \frac{Y}{Y + {Y}_{50 , B}} + {E}_{mαx , C} \times \\ \frac{Z}{Z + {Z}_{50 , C}} + {α}_{B C} \times {E}_{m α x , B} \times {E}_{mαx , C} \times \\ {\frac{Y}{Y + {Y}_{50 , B}} \times \frac{Z}{Z + {Z}_{50 , C}} )}^{{γ}_{int , B C}}\end{matrix}}{1 + {( \frac{Y}{{Y}_{50 , B}} + \frac{Z}{{Z}_{50 , C}} + {α}_{BC} \times \frac{Y}{{Y}_{50 , B}} \times \frac{Z}{{Z}_{50 , C}} )}^{{γ}_{int , B C}}} ] dXdZ \qquad ( XV )$$

wherein:
   drug concentrations Y′ and Y″ correspond to the concentrations of the 20^{th} and 80^{th} percentiles of the Y_{50,B} values obtained in said population of subjects each diagnosed with said disease, wherein Y_{50,B} was calculated for each subject in said population according to steps (a) to (e)(i);
   drug concentrations Z′ and Z″ correspond to the concentrations of the 20^{th} and 80^{th} percentiles of the Z_{50,C} values obtained in said population of subjects each diagnosed with said disease, wherein Z_{50,C} was calculated for each subject in said population according to steps (a) to (e)(i);
   E_{max,B}=maximum fractional decrease in LPC caused by drug B;
   E_{max,C}=maximum fractional decrease in LPC caused by drug C;
   Y_{50,B}=EC₅₀ concentration of drug C exerting half of E_{max,B};
   Z_{50,C}=EC₅₀ concentration of drug C exerting half of E_{max,C};
   Y=concentration of drug B;
   Z=concentration of drug C;

$${y}_{int , BC} = {γ}_{B} \times \frac{\frac{Y}{{Y}_{50 , B}}}{\frac{Y}{{Y}_{50 , B}} + \frac{Z}{{Z}_{50 , C}}} + ( 1 - \frac{\frac{Y}{{Y}_{50 , B}}}{\frac{Y}{{Y}_{50 , B}} + \frac{Z}{{Z}_{50 , C}}} ) \times {γ}_{C}$$

wherein:
   γB=steepness of the LCTi vs concentration curve for drug B;
   γC=steepness of the LCTi vs concentration curve for drug C;
and
α_{BC}=synergy parameter estimated from a two-drug surface interaction model determined by fitting the formula (XV′) to experimental values of LCTi counted according to step (d) after incubating sub-samples for said subject according to the steps referred to in (b)(iii) and (b)(iv) obtained for each concentration Y of drug B (where X=Z=0), the steps referred to in (b)(ix) and (b)(x) obtained for each concentration Z of drug C (where X=Y=0), and the steps referred to in b(xiv), b(xv) and b(xvi) obtained for each pair of concentrations of the combination of drug B and drug C:

$$L C T i = L C T {i}_{0 , BC} \times [ 1 - \frac{\begin{matrix}( {E}_{max , B} \times \frac{Y}{Y + {Y}_{50 , B}} + {E}_{max , C} \times \frac{Z}{Z + {Z}_{50 , C}} + \\ {α}_{B C} \times {E}_{max , B} \times {E}_{max , C} \times \\ {\frac{Y}{Y + {Y}_{50 , B}} \times \frac{Z}{Z + {Z}_{50 , C}} )}^{{γ}_{inc , BC}}\end{matrix}}{1 + {( \frac{Y}{{Y}_{50 , B}} + \frac{Z}{{Z}_{50 , C}} + {α}_{BC} \times \frac{Y}{{Y}_{50 , B}} \times \frac{Z}{{Z}_{50 , C}} )}^{{γ}_{inc , BC}}} ] . \qquad ( {XV}^{′} )$$

In an embodiment of each of said methods of the present invention, the normalised marker values determined in step (e)(ii) comprise NAUC_{A}, NAUC_{B} and/or NVUS_{AB}, wherein:
NAUC_{A} is a normalised value for AUC_{xy,A} which is calculated using the formula (VIII);

NAUC_{A}=100×AUC_{xy,A}/AUC_{max,A} (VIII)
NAUC_{B} is a normalised value for AUC_{xy,B} which is calculated using the formula (IX);

NAUC_{B}=100×AUC_{xy,B}/AUC_{max,B} (IX)
NVUS_{AB} is a normalised value for VUS_{AB} which is calculated using the formula (X);

NVUS_{AB}=100×VUS_{AB}/VUS_{max,AB} (X)
wherein:
AUC_{max,A}=the maximum value for AUC_{xy,A} obtained in a population of subjects each diagnosed with said disease, wherein AUC_{xy,A} was calculated for each subject in said population according to steps (a) to (e)(ii);
AUC_{max,B}=the maximum value for AUC_{xy,B} obtained in said population of subjects each diagnosed with said disease, wherein AUC_{xy,B} was calculated for each subject in said population according to steps (a) to (e)(ii); and
VUS_{max,AB}=the maximum value for VUS_{AB} obtained in said population of subjects each diagnosed with said disease, wherein VUS_{AB} was calculated for each subject in said population according to steps (a) to (e)(ii).

In a more preferred embodiment of each of said methods of the present invention, when said combination of drugs additionally comprises drug C, the normalised marker values determined in step (e)(iii) optionally further comprise NAUC_{C}, NVUS_{AC} and/or NVUS_{BC}, wherein:
NAUC_{C} is a normalised value for AUC_{xy,C} which is calculated using the formula (XVI);

NAUC_{C}=100×AUC_{xy,C}/AUC_{max,C} (XVI)
NVUS_{AC} is a normalised value for VUS_{AC} which is calculated using the formula (XVII);

NVUS_{AC}=100×VUS_{AC}/VUS_{max,AC} (XVII)

NVUS_{BC} is a normalised value for VUS_{BC} which is calculated using the formula (XVIII);

NVUS_{BC}=100×VUS_{BC}/VUS_{max,BC} (XVIII)

wherein:
AUC_{max,C}=the maximum value for AUC_{xy,C} obtained in said population of subjects each diagnosed with said disease, wherein AUC_{xy,C} was calculated for each subject in said population according to steps (a) to (e)(ii);
VUS_{max,AC}=the maximum value for VUS_{AC} obtained in said population of subjects each diagnosed with said disease, wherein VUS_{AC} was calculated for each subject in said population according to steps (a) to (e)(ii); and
VUS_{max,BC}=the maximum value for VUS_{BC} obtained in said population of subjects each diagnosed with said disease, wherein VUS_{BC} was calculated for each subject in said population according to steps (a) to (e)(ii).

In an embodiment of each of the systems of the present invention, the means for carrying out the step of determining pharmacodynamic parameter values, activity marker values and normalized marker values comprises at least one computer program product.

In the present invention a computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may include, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and kits according to embodiments and/or steps of the invention. It will be understood that each square or diamond-shaped block of the flowchart illustrations and/or block diagrams, and combinations of blocks in the flowchart illustrations and/or block diagrams, can be implemented by biological techniques or computer readable program instructions, or combinations thereof.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Both of said methods of the present invention also comprise a step (f) of selecting:

(i) the pharmacodynamic parameter value or values determined according to step (e)(i) for each subject in said population of subjects; and/or

(ii) the activity marker value or values determined according to step (e)(ii) for each subject in said population of subjects; and/or

(iii) normalized marker value or values determined according to step (e)(iii) for each subject in said population of subjects, and/or

(iv) a clinical variable value or values for each subject in said population of subjects,

which are dependent on clinical resistance or clinical sensitivity to said combination of drugs, whereby a value is dependent when the probability of said value being independent from clinical resistance or clinical sensitivity is less than or equal to 0.05. A value is dependent on clinical resistance or clinical sensitivity when the probability of said value being independent from clinical resistance or clinical sensitivity determined to be less than or equal to 0.05 using statistical independence tests.

A clinical variable is a variable that is different from any of said pharmacodynamic parameter values, said activity marker values, or said normalized marker values. Said clinical variable may be selected from the group of subject age, sex, race, blood type, presenting leukocyte count, presence of mutations in the NPM1 or FLT3 genes, cytogenetic risk group, whether said subject has or has not undergone first line treatment for said disease, whether said subject has or has not undergone second line treatment for said disease, count of mononuclear cells obtained from peripheral blood (WBC in PB), Eastern Cooperative Oncology Group (ECOG) performance status, type of haematological cancer (French-American-British (FAB) classification, de novo vs. secondary AML), karyotype, hematological response, number of 3+7 induction cycles, date of response, date of last follow-up, and whether said subject has or has not undergone post-remission therapy. Preferably said clinical variable is selected from the group of subject age, sex, race, blood type, presenting leukocyte (WBC) count, presence of mutations in the NPM1 or FLT3 genes, cytogenetic risk group, whether said subject has or has not undergone first line treatment for said disease, whether said subject has or has not undergone second line treatment for said disease. More preferably said clinical variable is selected from the group of age at diagnosis, sex, cytogenetic risk group, % blast at diagnosis, WBC count, FAB subtype, ECOG performance status, presence of mutations in the NPM1 gene, and presence of mutations in the FLT3 gene. Said clinical variable value describes each subject in said population of subjects with said disease and may be a continuous number or a whole number, depending on the variable thus described. For example, when a subject has undergone first line treatment for said disease said clinical variable value may be 0 and when a subject has not undergone first line treatment for said disease said clinical variable value may be 1, or vice versa.

In an embodiment of each of the systems of the present invention, the means for carrying out the step (f) of selecting comprises at least one computer program product, wherein said computer program product may be different from that used in step (e).

Thus, in a particularly preferred embodiment of the methods of the present invention, the steps (a) to (f) respectively comprise the following:
(a) separating a tissue sample obtained from said subject into between 20 and 30 sub-samples;
(b) carrying out the steps of:
   (i) incubating a sub-sample for a time T of between 36 and 72 hours in the presence of said drug A at a concentration X; and
   (ii) repeating step (b)(i) an additional (N−1) times, each time with a different sub-sample using a value for X that is different from that used in previous repetitions of step (b)(i);
   wherein N is a whole number selected from between 5 and 10, inclusive;
   and
   (iii) incubating a sub-sample for said time Tin the presence of said drug B at a concentration Y; and
   (iv) repeating step (b)(iii) an additional (M−1) times, each time with a different sub-sample using a value for Y that is different from that used in previous repetitions of step (b)(iii);
   wherein M is a whole number selected from between 5 and 10, inclusive;
   and
   (v) incubating a sub-sample for said time T in the presence of a combination of drugs comprising said drug A and said drug B, wherein
      the concentration of said drug A is a concentration X corresponding to the concentration at the percentile value P_{Hα,A} from the distribution of X_{50,A} values obtained in said population of subjects each diagnosed with said disease, wherein percentile value P_{Hα,A} is calculated by the formula (A):

P_{Hα,A}=cos(α°)×H (A)
wherein:
   H corresponds to a reference percentile selected from the group of
   10,

$$[ 10 + \frac{80}{R - 1} \times ( r - 1 ) ]$$

and 90, wherein:
   r is a whole number selected from between 2 and (R−1), inclusive
α is in degrees and is calculated from the formula:

$$α = \frac{90}{( W + 1 )} \times w$$

wherein:
   w is a whole number selected from between 1 and W, inclusive the concentration of said drug B is a concentration Y corresponding to the concentration at the percentile value P_{Hα,B} from the distribution of Y_{50,B} values obtained in said population of subjects each diagnosed with said disease, wherein percentile value P_{Hα,B} is calculated by the formula (B):

P_{Hα,B}=cos(90°−α°)×H (B)
(vi) repeating step (b)(v) an additional (R−1) times, each time with a different sub-sample using a value for H that is different from that used in previous repetitions of step (b)(v), and using the same value for w that is used in step (b)(v); and
(vii) repeating steps (b)(v) and (b)(vi) an additional (W−1) times, each time with a different sub-sample using a value for w that is different from that used in previous repetitions of steps (b)(v) and (b)(vi);
wherein;
   R is 3;
   W is 3,
and wherein:
   X_{50,A} is the concentration of drug A exerting half of the maximum activity in a subject, estimated according to step (e)(i), below;
   Y_{50,B} is the concentration of drug B exerting half of the maximum activity in a subject, estimated according to step (e)(i), below;
and
(viii) incubating a sub-sample for said time T;
(c) (i) adding at least one conjugated antibody to each sub-sample incubated in step (b) to identify at least one pathological cell-type therein; and
   (ii) adding at least one cell death or apoptosis marker to each sub-sample incubated in step (b) to identify apoptotic cells therein;
(d) counting the number of live cells (LCTi) of each cell-type identified in step (c) which remain after incubation of each sub-sample according to step (b) by counting the number of cells of said at least one pathological cell-type identified according to step (c)(i) which are not identified as apoptotic according to step (c)(ii);
(e) determining for each cell-type identified in step (c):
   (i) pharmacodynamic parameter values comprising an X_{50,A} value, a LCTi_{0,A} value, an E_{max,A} value, a γ_{A} value, a Y_{50,B} value, an LCTi_{0,B} value, an E_{max,B} value and a γ_{B} value, wherein:
      said X_{50,A}, LCTi_{0,A}, E_{max,A} and γ_{A} values are estimated from a single drug dose-response pharmacodynamic mixed effects non-linear population model determined by fitting the formula (I) to experimental values of LCTi counted according to step (d) after incubating sub-samples for each subject in a population according to steps (b)(i) and (b)(ii) obtained for each concentration X of drug A:

$$L C T i = {LCTi}_{0 , A} \times [ 1 - {E}_{max , A} \times \frac{{X}^{{γ}_{A}}}{{X}^{{γ}_{A}} + {X}_{50 , A}^{{γ}_{A}}} ] \qquad ( I )$$

said Y_{50,B}, LCTi_{0,B}, E_{max,B} and γ_{B} values are estimated from a single drug dose-response pharmacodynamic mixed effects non-linear population model determined by fitting the formula (II) to experimental values of LCTi counted according to step (d) after incubating sub-samples for each subject in said population according to steps (b)(iii) and (b)(iv) obtained for each concentration Y of drug B:

$$L C Ti = {LCTi}_{0 , B} \times [ 1 - {E}_{max , B} \times \frac{{Y}^{{γ}_{B}}}{{Y}^{{γ}_{B}} + {Y}_{50 , B}^{{γ}_{B}}} ] \qquad ( II )$$

wherein said population comprises said subject and other subjects diagnosed with said disease;
wherein:
   X=concentration of drug A;
   X_{50,A} is the concentration of drug A exerting half of maximum activity;
   LCTi_{0,A} is the basal (pre-incubation) number of LCTi and is equal to the LCTi counted after incubating a sub-sample in the absence of a drug according to the step referred to in (b)(viii);
   E_{max,A}, is the maximum fractional decrease of LCTi_{0,A} caused by drug A;
   γ_{A} is the steepness of the LCTi vs concentration curve for drug A;
   Y=concentration of drug B;
   Y_{50,B} is the concentration of drug B exerting half of maximum activity;
   LCTi_{0,B} is the basal (pre-incubation) number of LCTi and is equal to the LCTi counted after incubating a sub-sample in the absence of a drug according to the step referred to in (b)(viii);
   E_{max,B}, is the maximum fractional decrease of LCTi_{0,B} caused by drug B;
   γ_{B} is the steepness of the LCTi vs concentration curve for drug B;
(ii) activity marker values comprising an AUC_{xy,A} value, an AUC_{xy,B} value, an α_{AB} value and a VUS_{AB} value, wherein:
   said AUC_{xy,A} value is calculated using the formula (III):

AUC_{xy,A}=AUC_{x,A}−A_{y:10-90,A} (III)
wherein:
said AUC_{x,A} value is the integral between two drug concentrations X′ and X″ of a function derived from formula (I) for the % survival after incubating sub-samples according to steps (b)(i) and (b)(ii) obtained for each concentration X of drug A, wherein LCTi_{0,A} is considered as 100% survival, and is calculated using the formula (IV):

$$A U {C}_{x , A} = {\int }_{{X}^{′}}^{{X}^{″}} 100 \times [ 1 - {E}_{max , A} \times \frac{{X}^{{γ}_{A}}}{{X}^{{γ}_{A}} + {X}_{50 , A}^{{γ}_{A}}} ] d X \qquad ( IV )$$

wherein drug concentrations X′ and X″ correspond to the concentrations of the 20^{th} and 80^{th} percentiles of the X_{50,A} values obtained in said population of subjects each diagnosed with said disease, wherein X_{50,A} was calculated for each subject in said population according to steps (a) to (e)(i);
and
said A_{y:10-90,A} value is the surface from AUC_{x,A} that falls outside the 10% and 90% boundaries of the % survival, wherein LCTi_{0,A} is considered as 100% survival;
and
said AUC_{xy,B} value is calculated using the formula (V):

AUC_{xy,B}=AUC_{x,B}−A_{y:10-90,B} (V)
wherein:
said AUC_{x,B} value is the integral between two drug concentrations Y′ and Y″ of a function derived from formula (II) for the % survival after incubating sub-samples according to steps (b)(iii) and (b)(iv) obtained for each concentration Y of drug B, wherein LCTi_{0,B} is considered as 100% survival, and is calculated using the formula (VI):

$${AUC}_{x , B} = {\int }_{{Y}^{′}}^{{Y}^{″}} 100 \times [ 1 - {E}_{max , B} \times \frac{{Y}^{{γ}_{B}}}{{Y}^{{γ}_{B}} + {Y}_{50 , B}^{{γ}_{B}}} ] dY \qquad ( VI )$$

wherein drug concentrations Y′ and Y″ correspond to the concentrations of the 20th and 80th percentiles of the Y_{50,B} values obtained in said population of subjects each diagnosed with said disease, wherein Y_{50,B} was calculated for each subject in said population according to steps (a) to (e)(i);
and
said A_{y:10-90,B} value is the surface from AUC_{x,B} that falls outside the 10% and 90% boundaries of the % survival, wherein LCTi_{0,A} is considered as 100% survival;
and
said VUS_{AB} value is calculated using the formula (VII), wherein said VUS_{AB} value is the double integral between two drug concentrations X′ and X″ for drug A and two drug concentrations Y′ and Y″ for drug B of the model function of the natural log of LCTi counted after incubating sub-samples according to steps (b)(v) to (b)(vii), wherein LCTi_{0,A}=LCTi_{0,B} and is considered as 100% survival, and is calculated using the formula (VII),

$${VUS}_{AB} = \\ {\int }_{{X}^{′}}^{{X}^{″}} {\int }_{{Y}^{′}}^{{Y}^{″}} 100 \times [ 1 - \frac{{( \begin{matrix}\begin{matrix}{E}_{max , A} \times \\ \frac{X}{X + {X}_{50 , A}} + {E}_{mαx , B} \times \end{matrix} \\ \frac{Y}{Y + {Y}_{50 , B}} + {α}_{A B} \times \\ \begin{matrix}{E}_{max , A} \times {E}_{mαx , B} \times \\ \frac{X}{X + {X}_{50 , A}} \times \frac{Y}{Y + {Y}_{50 , B}}\end{matrix}\end{matrix} )}^{{γ}_{int , AB}}}{1 + {( \begin{matrix}\frac{X}{{X}_{50 , A}} + \frac{Y}{{Y}_{50 , B}} + {α}_{AB} \times \\ \frac{X}{{X}_{50 , A}} \times \frac{Y}{{Y}_{50 , B}}\end{matrix} )}^{{γ}_{int , AB}}} ] dX dY \qquad ( VII )$$

wherein:
   drug concentrations X′ and X″ correspond to the concentrations of the 20^{th} and 80^{th} percentiles of the X_{50,A} values obtained in said population of subjects each diagnosed with said disease, wherein X_{50,A} was calculated for each subject in said population according to steps (a) to (e)(i);
   drug concentrations Y′ and Y″ correspond to the concentrations of the 20^{th} and 80^{th} percentiles of the Y_{50,B} values obtained in said population of subjects each diagnosed with said disease, wherein Y_{50,B} was calculated for each subject in said population according to steps (a) to (e)(i);
   E_{max,A}=maximum fractional decrease in LPC caused by drug A;
   E_{max,B}=maximum fractional decrease in LPC caused by drug B;
   X_{50,A}=EC₅₀ concentration of drug A exerting half of E_{max,A};
   Y_{50,B}=EC₅₀ concentration of drug B exerting half of E_{max,B};
   X=concentration of drug A;
   Y=concentration of drug B;

$${Y}_{int , AB} = {γ}_{A} \times \frac{\frac{X}{{X}_{50 , A}}}{\frac{X}{{X}_{50 , A}} + \frac{Y}{{Y}_{50 , B}}} + ( 1 - \frac{\frac{X}{{X}_{50 , A}}}{\frac{X}{{X}_{50 , A}} + \frac{Y}{{Y}_{50 , B}}} ) \times {γ}_{B}$$

wherein:
   γA=steepness of the LCTi vs concentration curve for drug A;
   γB=steepness of the LCTi vs concentration curve for drug B; and
α_{AB}=synergy parameter estimated from a two-drug surface interaction model determined by fitting the formula (VII′) to experimental values of LCTi counted according to step (d) after incubating sub-samples for said subject according to the steps referred to in (b)(i) and (b)(ii) obtained for each concentration X of drug A, the steps referred to in (b)(iii) and (b)(iv) obtained for each concentration Y of drug B, and the steps referred to in b(v), b(vi) and b(vii) obtained for each pair of concentrations of the combination of drug A and drug B:

$$LCTi = {LCTi}_{0 , AB} \times [ 1 - \frac{{( \begin{matrix}\begin{matrix}\begin{matrix}{E}_{max , A} \times \frac{X}{X + {X}_{50 , A}} + {E}_{max , B} \times \\ \frac{Y}{Y + {Y}_{50 , B}} + {α}_{AB} \times {E}_{max , A} \times \end{matrix} \\ {E}_{max , B} \times \end{matrix} \\ \frac{X}{X + {X}_{50 , A}} \times \frac{Y}{Y + {Y}_{50 , B}}\end{matrix} )}^{{γ}_{int , AB}}}{1 + {( \begin{matrix}\frac{X}{{X}_{50 , A}} + \frac{Y}{{Y}_{50 , B}} + {α}_{AB} \times \\ \frac{X}{{X}_{50 , A}} \times \frac{Y}{{Y}_{50 , B}}\end{matrix} )}^{{Y}_{int , AB}}} ] \qquad ( {VII}^{′} )$$

(iii) normalized marker values comprising NAUC_{A}, NAUC_{B} and NVUS_{AB}, wherein:
   NAUC_{A} is a normalised value for AUC_{xy,A} which is calculated using the formula (VIII);

NAUC_{A}=100×AUC_{xy,A}/AUC_{max,A} (VIII)
NAUC_{B} is a normalised value for AUC_{xy,B} which is calculated using the formula (IX);

NAUC_{B}=100×AUC_{xy,B}/AUC_{max,B} (IX)
NVUS_{AB} is a normalised value for VUS_{AB} which is calculated using the formula (X);

NVUS_{AB}=100×VUS_{AB}/VUS_{max,AB} (X)
wherein:
   AUC_{max,A}=the maximum value for AUC_{xy,A} obtained in a population of subjects each diagnosed with said disease, wherein AUC_{xy,A} was calculated for each subject in said population according to steps (a) to (e)(ii);
   AUC_{max,B}=the maximum value for AUC_{xy,B} obtained in said population of subjects each diagnosed with said disease, wherein AUC_{xy,B} was calculated for each subject in said population according to steps (a) to (e)(ii); and
   VUS_{max,AB}=the maximum value for VUS_{AB} obtained in said population of subjects each diagnosed with said disease, wherein VUS_{AB} was calculated for each subject in said population according to steps (a) to (e)(ii);
(f) selecting:
   (i) the pharmacodynamic parameter value or values determined according to step (e)(i) for each subject in said population of subjects; and/or
   (ii) the activity marker value or values determined according to step (e)(ii) for each subject in said population of subjects; and/or
   (iii) normalized marker value or values determined according to step (e)(iii) for each subject in said population of subjects, and/or
   (iv) a clinical variable value or values for each subject in said population of subjects, which are dependent on clinical resistance or clinical sensitivity to said combination of drugs, whereby a value is dependent when the probability of said value being independent from clinical resistance or clinical sensitivity is less than or equal to 0.05,
   wherein each of said clinical variable values is selected from the group of age at diagnosis, sex, cytogenetic risk group, % blast at diagnosis, WBC count, FAB subtype, ECOG performance status, presence of mutations in the NPM1 gene, and presence of mutations in the FLT3 gene;

wherein:
said combination of drugs comprises 2 or 3 drugs, wherein
   drug A is cytarabine and drug B is idarubicin; and
   said combination of drugs optionally comprises an additional drug C selected from the group consisting of fludarabine, etoposide, thioguanine and clofarabine;
said tissue is bone-marrow; and
said disease is acute myeloid leukemia.

The method for determining the efficacy of treatment with a combination of drugs comprising a drug A and a drug B in a subject diagnosed with a disease additionally comprises further subsequent steps (g), (h), (j) and (k). In contrast, the method for classifying the utility of drug combinations comprising a drug A and a drug B in treatment of a subject diagnosed with a disease additionally comprises further subsequent steps (g′), (h′) and (j′).

In the method for determining the efficacy of treatment with a combination of drugs comprising a drug A and a drug B in a subject diagnosed with a disease, the step (g) comprises creating a response function using a generalized linear model (GLM) or a generalized additive model (GAM) for said combination of drugs for said population of subjects using at least one of the values which were selected in step (f), wherein the receiver operating characteristic (ROC) curve derived from said model function has an area under the curve which is equal to or greater than 0.8, and the lower limit of the 95% confidence interval of said area under the curve is greater than 0.5. In an embodiment of the system for determining the efficacy of treatment with a combination of drugs comprising a drug A and a drug B in a subject diagnosed with a disease, the means for carrying out the step (g) of creating a response function comprises at least one computer program product.

In the method for determining the efficacy of treatment with a combination of drugs comprising a drug A and a drug B in a subject diagnosed with a disease, the step (h) comprises calculating a threshold limit of the response function created in step (g) from the point on said receiver operating characteristic curve:
at which sensitivity and specificity are maximized and equal in value (maxSpSe); or
at which specificity is prioritized over sensitivity (mMCT); or
which is closest to the (1,0) coordinate plane (geometric).

Preferably sensitivity and specificity are maximized and equal in value. However, a reduced threshold limit (achieved when specificity is prioritized over sensitivity) would result in fewer false negatives (and more false positives), corresponding to a rightward movement on the ROC curve. In an embodiment of the system for determining the efficacy of treatment with a combination of drugs comprising a drug A and a drug B in a subject diagnosed with a disease, the means for carrying out the step (h) of calculating a threshold limit of the response function comprises at least one computer program product.

In the method for determining the efficacy of treatment with a combination of drugs comprising a drug A and a drug B in a subject diagnosed with a disease, the step (j) comprises calculating a S/R value (i.e. sensitivity/resistance value indicative of the clinical sensitivity/resistance) for said combination of drugs against said disease in said subject using the response function created in step (g) and:
(i) the pharmacodynamic parameter value or values determined according to step (e)(i) for said subject; and/or
(ii) the activity marker value or values determined according to step (e)(ii) for said subject; and/or
(iii) the normalized marker value or values determined according to step (e)(iii) for said subject, and/or
(iv) the clinical variable value or values for said subject, which are variables in said response function. In other words, an S/R value for said combination of drugs against said disease in said subject is calculated by introducing the values referred to under (j)(i) to (j)(iv) for said subject into said response function, where relevant. In an embodiment of the system for determining the efficacy of treatment with a combination of drugs comprising a drug A and a drug B in a subject diagnosed with a disease, the means for carrying out the step (j) of calculating a S/R value comprises at least one computer program product.

In the method for determining the efficacy of treatment with a combination of drugs comprising a drug A and a drug B in a subject diagnosed with a disease, the step (k) comprises determining the efficacy of treatment of said disease in said subject with said combination of drugs by comparing the S/R value calculated in step (j) with the threshold limit calculated in step (h), wherein
when said S/R value is equal to or greater than said threshold limit, said disease is sensitive to treatment with said combination of drugs in said subject; and
when said S/R value is less than said threshold limit, said disease is resistant to treatment with said combination of drugs in said subject. Moreover, when said S/R value is equal to or greater than said threshold limit and the difference between said S/R value and said threshold value is large (and positive), said disease is more sensitive to treatment with said combination of drugs in said subject than it is when said S/R value is equal to or greater than said threshold limit and the difference between said S/R value and said threshold value is small (and positive) or zero. Similarly, when said S/R value is less than said threshold limit and the difference between said S/R value and said threshold value is large (and negative), said disease is more resistant to treatment with said combination of drugs in said subject than it is when said S/R value is less than said threshold limit and the difference between said S/R value and said threshold value is small (and negative). In an embodiment of the system for determining the efficacy of treatment with a combination of drugs comprising a drug A and a drug B in a subject diagnosed with a disease, the means for carrying out the step (k) of determining the efficacy of treatment of said disease in said subject with said combination of drugs comprises at least one computer program product.

Thus, in one embodiment of the system for determining the efficacy of treatment with a combination of drugs comprising a drug A and a drug B in a subject diagnosed with a disease:
the means for carrying out the step of separating a tissue sample comprises a microfluidic stem cell separation device;
the means for carrying out the steps referred to in (b) of incubating comprises a cell culture incubator;
the means for carrying out the step of adding at least one marker comprises a pipette or injector;
the means for carrying out the steps of counting the number of live cells comprises a cytometer;
the means for carrying out the step of determining pharmacodynamic parameter values, activity marker values and normalized marker values comprises at least one computer program product;
the means for carrying out the step of selecting comprises at least one computer program product;
means for carrying out a step of creating a response function comprises at least one computer program product;
means for carrying out a step of calculating a threshold limit of the response function comprises at least one computer program product;
means for carrying out a step of calculating a S/R value comprises at least one computer program product; and
the means for determining the efficacy of treatment of said disease in said subject with said combination of drugs comprises at least one computer program product.

In the method for classifying the utility of drug combinations comprising a drug A and a drug B in treatment of a subject diagnosed with a disease, the step (g′) comprises calculating a score S for treatment of said subject with said drug A and said drug B, wherein said score corresponds with or is calculated using at least one of the values which were selected in step (f). In a preferred embodiment of said method for classifying the utility of drug combinations, said score S:

(i) is calculated in step (g′) using the formula (XIX):

$$S = ( \frac{{Σ ( NAUC )}_{d} + {Σ ( NVUS )}_{cd}}{D + C} ) \times f \qquad ( XIX )$$

wherein:
(NAUC)_{d}=normalised value NAUC for each drug included in said treatment;
(NVUS)_{cd}=normalised value NVUS for each combination of drugs included in said treatment;
D=number of drugs included in said treatment;
C=number of combinations of drugs included in said treatment; and
f=compensation factor for multiple drugs treatments, wherein:

$$f = ( 1 - ( \frac{( 3 - D )}{10} ) )$$

or

(ii) is a value for NVUS selected from (NVUS)_{cd} S=NVUS_{AB} or, when said combination of drugs comprises a drug A, a drug B and a drug C, S is selected from NVUS_{AB}, NVUS_{AC} and NVUS_{BC}).

In an embodiment of the system for classifying the utility of drug combinations comprising a drug A and a drug B in treatment of a subject diagnosed with a disease, the means for carrying out the step (g′) of calculating a score comprises at least one computer program product

In the method for classifying the utility of drug combinations comprising a drug A and a drug B in treatment of a subject diagnosed with a disease, the step (h′) comprises carrying out steps (b) to (f) and (g′) for each combination of drugs to be classified. In an embodiment of the system for classifying the utility of drug combinations comprising a drug A and a drug B in treatment of a subject diagnosed with a disease, the means for carrying out the steps referred to in (h) are the same as those according to the steps referred to in (a) to (f) and (g′).

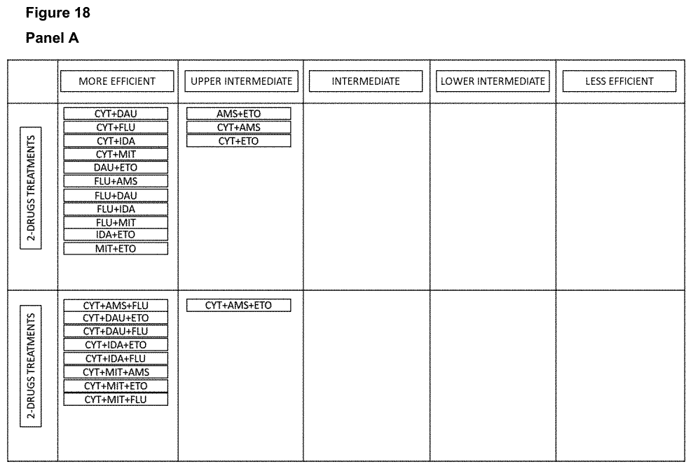

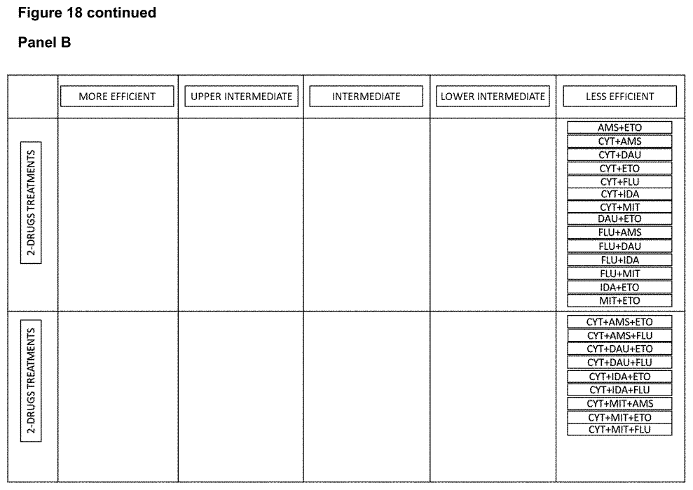

In the method for classifying the utility of drug combinations comprising a drug A and a drug B in treatment of a subject diagnosed with a disease, the step (j′) comprises classifying each combination of drugs using the score determined in steps (g′) and (h′), whereby a combination of drugs having a score of:
(i) greater than 80 is assigned to classification category I having a classification value of 2;
(ii) less than or equal to 80 and greater than 60 is assigned to classification category II having a classification value of 1;
(iii) less than or equal to 60 and greater than 40 is assigned to classification category III having a classification value of 0;
(iv) less than or equal to 40 and greater than 20 is assigned to classification category IV having a classification value of −1; or
(v) less than or equal to 20 is assigned to classification category V having a classification value of −2,
whereby:
each combination of drugs which is assigned to a classification category having a positive classification value or a classification value of zero is of highest or greatest utility in treatment of a subject diagnosed with a disease; and
each combination of drugs which is assigned to a classification category having a negative classification value is of lowest or least utility in treatment of a subject diagnosed with a disease. Each combination of drugs which is assigned to a classification category having a positive classification value or a classification value of zero is that to which said disease is predicted to be sensitive in said subject. Analogously, each combination of drugs which is assigned to a classification category having a negative classification value is that to which said disease is predicted to be resistant in said subject. Preferably, each combination of drugs which is assigned to a classification category having a positive classification value is of greatest utility in treatment of a subject diagnosed with a disease and each combination of drugs which is assigned to classification category having a negative classification value is of least utility in treatment of a subject diagnosed with a disease. More preferably, each combination of drugs which is assigned to the classification category of highest positive value (i.e. classification category I) is of greatest utility in treatment of a subject diagnosed with a disease. In a particularly preferred embodiment, each combination of drugs having a score of greater than 80, more preferably greater than 85, even more preferably greater than 90, is that which is assigned to the classification category of highest positive value (i.e. classification category I) which is of greatest utility in treatment of a subject diagnosed with a disease and a combination of drugs belonging to this classification category, as determined by the method and system for classifying the utility of combinations of drugs, is selected in a plan of care for prescription in a method of treatment. Each combination of drugs falling into this category is that to which said disease is predicted to be most sensitive in said subject. Analogously, each combination of drugs which is assigned to the classification category of lowest negative value (i.e. classification category V) is of least utility in treatment of a subject diagnosed with a disease. Each combination of drugs falling into this category is that to which said disease is predicted to be most resistant in said subject. Thus, classification category I corresponds with that classification category which is “more efficient” in treatment of said subject diagnosed with said disease, classification category II corresponds with that classification category which is “upper intermediate” in efficacy of treatment of said subject diagnosed with said disease, classification category III corresponds with that classification category which is “intermediate” in efficacy of treatment of said subject diagnosed with said disease, classification category IV corresponds with that classification category which is “lower intermediate” in efficacy of treatment of said subject diagnosed with said disease, and classification category V corresponds with that classification category which is “less efficient” in efficacy of treatment of said subject diagnosed with said disease (FIG. 18).

Classifying is, alternatively, such that a combination of drugs having a score of:
(i) greater than (100−JJ) is assigned to classification category I′ having a classification value of 1;
(ii) less than or equal to (100−JJ) and greater than JJ is assigned to classification category III′ having a classification value of 0; or
(iii) less than or equal to JJ is assigned to classification category V′ having a classification value of −1,

wherein JJ is a value selected from between 5 and 30, preferably wherein JJ is a value selected from between 10 and 20,

wherein each combination of drugs which is assigned to a classification category having a positive classification value is of greatest utility in treatment of a subject diagnosed with a disease and each combination of drugs which is assigned to classification category having a negative classification value is of least utility in treatment of a subject diagnosed with a disease. In this alternative form of classifying, classification category I′ corresponds with classification category I disclosed above, classification category III′ corresponds with classification categories II, III and IV disclosed above, and classification category V′ corresponds with classification category V disclosed above.

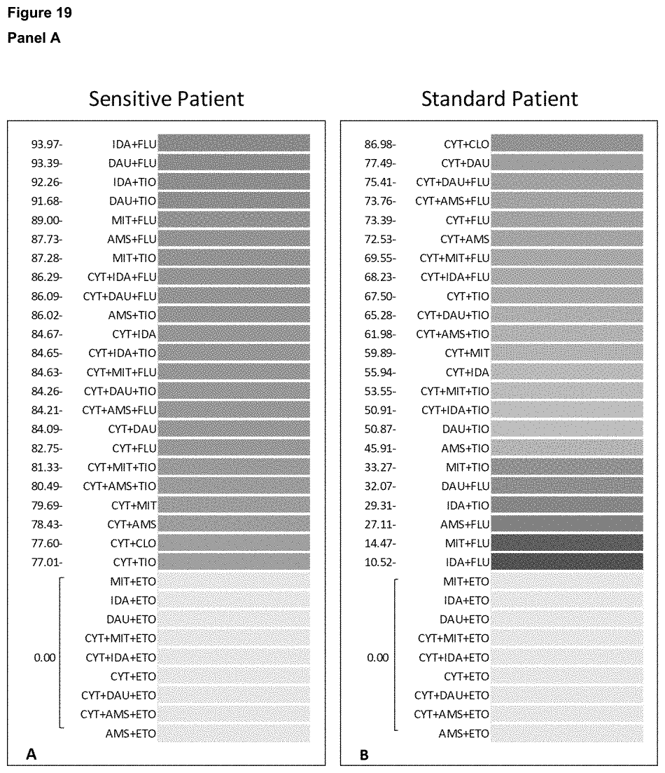

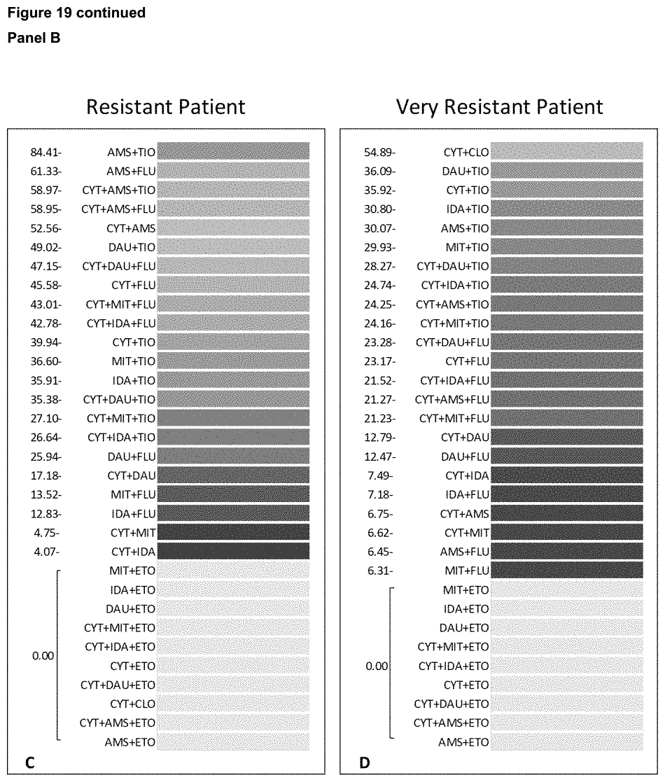

In an alternative to step (j′), the method for classifying the utility of drug combinations comprising a drug A and a drug B in treatment of a subject diagnosed with a disease may comprise step (j″) which comprises ranking each combination of drugs in order of score determined in steps (g′) and (h′), whereby the combination of drugs having highest score S is that which is of greatest utility in treatment of a subject diagnosed with a disease (FIG. 19).

In an embodiment of the system for classifying the utility of drug combinations comprising a drug A and a drug B in treatment of a subject diagnosed with a disease, the means for classifying each combination of drugs in order of score comprises at least one computer program product.

Thus, in one embodiment of the system for classifying the utility of drug combinations comprising a drug A and a drug B in treatment of a subject diagnosed with a disease:
the means for carrying out the step of separating a tissue sample comprises a microfluidic stem cell separation device;
the means for carrying out the steps referred to in (b) of incubating comprises a cell culture incubator;
the means for carrying out the step of adding at least one marker comprises a pipette or injector;
the means for carrying out the steps of counting the number of live cells comprises a cytometer;
the means for carrying out the step of determining pharmacodynamic parameter values, activity marker values and normalized marker values comprises at least one computer program product;
the means for carrying out the step of selecting comprises at least one computer program product;
the means for carrying out the step of calculating a score comprises at least one computer program product; and
the means for classifying each combination of drugs in order of score comprises at least one computer program product;

wherein the means for carrying out the steps referred to in (h′) are the same as those according to the steps referred to in (a) to (g′).

Note that the steps for determining the activity markers (AUC and VUS) disclosed in step (e)(ii) of the present invention comprise the steps of a method to limit the calculation of said activity markers to the concentration range and activity range that yields the most informative results (namely AUCxy). When limit the calculation of said activity markers to the concentration range and activity range that yields the most informative results, an improved correlation was achieved over that obtained when calculation of said activity markers used the entire concentration and activity ranges.

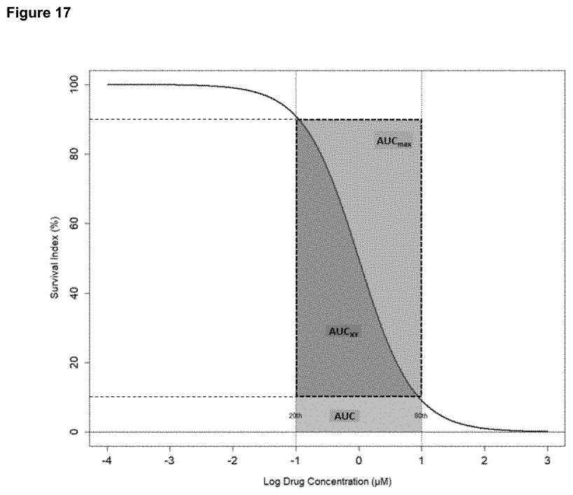

For the drug concentration range, such limits are defined by statistical values from the distribution of results of the whole population and correspond to the 20th and 80th percentiles of the EC₅₀ values. For the activity range (% survival), normalized values are restricted to the interval between the 10% and 90%. These limits are shown in FIG. 17, wherein the rectangle bounded by said limits represents that upon which correlation was effected.

Thus, the present invention also discloses a normalization method to apply to AUCxy values as a reference to a benchmark area that is defined by a rectangle described by the same limits mentioned above that are defined by the whole population results and the 10%-90% response interval. The output value NAUCxy (i.e. N_AUCxy) has a range between 0 and 100%, and is equivalent to a percentile value range, however it overcomes the limitation of the range percentile when it is calculated in populations which do not follow a symmetric normal distribution. For instance, in very asymmetric distributions, minimal variations of activity might imply huge variations in range percentiles. Distribution of clinical responses to cytotoxic treatments, especially in first line patients, are commonly very asymmetric showing higher tendency towards sensitive responses.

This normalization provides a standard value of the response of a patient sample to a treatment, by considering the behaviour of the whole population as a benchmark. Moreover, this value enables classification of the activities observed for multiple treatments. Classification of the activity allows an estimation of each treatment efficacy and a rough comparison among treatments to be provided. The criteria to classify activities may vary from just a number of equidistant range intervals or intervals defined by any other criteria, including for instance references to known response rates.

Thus, this normalization enables to generate the classification of multiple treatments that can be recommended, as a therapeutic guide, for each individual patient. To achieve the aforementioned classification, there are several factors to consider, such as activity, sensitivity, clinical efficacy and toxicity. The activity of drugs and combination treatments can be assessed by calculating AUCs and synergism. The result is that a spectrum of activities ranging from higher to lower exists across patient samples for every treatment. Samples showing higher activity represent patients who are determined as more sensitive for that treatment. Conversely, samples showing lower activity represent patients who are determined as more resistant for that treatment. In other words, the activity reflects the sensitivity or resistance of a patient towards a given treatment. It is helpful to define a threshold to categorize patients as either sensitive, undetermined, or resistant. For this threshold, the clinical efficacy of the treatment is relevant; for instance, in acute myeloid leukemia (AML) the standard 1st line treatment of cytarabine+idarubicin (CYT+IDA) achieves complete remission (a good response) in 70% of the patients. Hence, a prediction algorithm may identify approximately 70% of 1st line patients as sensitive and approximately a 30% of patients resistant, as shown in Example 1 (CYT-IDA correlation). Alternatively, a simpler scoring based on AUCs and synergies without clinical correlation to validate the threshold, may arbitrarily define safe extreme thresholds, e.g. the 20% samples showing the highest activity score are estimated to predict highly sensitive patients for that treatment. Preferably the 20% of samples showing the highest activity score are those falling into classification category I having a score of greater than 80, more preferably the 15% of samples showing the highest activity score are those falling into classification category I having a score of greater than 85, even more preferably the 10% of samples showing the highest activity score are those falling into classification category I having a score of greater than 90, and are estimated to predict patients which are highly sensitive to those treatments having a score falling into said classification category I. Similarly, the 20% samples showing the lowest activity score are estimated to predict highly resistant patients for that same treatment.

The prediction of sensitive vs resistant patients described above, identifying patient most sensitive to a given treatment, only makes sense within the same treatment. Comparison of such classifying of activity score with another different treatment is difficult and often leads to confusion. For example, in AML there several cytotoxic drugs of similar average activity, but many treatments combine 2 drugs and others 3 drugs that include these 2 drugs plus a 3rd drug. If we assume similar doses for all 3 drugs, then the 3-drug treatment includes precisely the 2-drug treatment adding an extra 3rd drug. When the 3rd drug has good or substantial activity, the 3-drug treatment has more cytotoxic power than the 2-drug treatment. Yet the classifying of sensitivity for both treatments does not reflect this fact. On the contrary, the sensitivity estimation penalizes the 3-drug treatment, because if the sensitivity of the 3rd drug is lower than the other 2 drugs then the overall 3-drug sensitivity will be lower than the 2 drug treatment, yet the 3 drug treatment is stronger. This example shows that the measurement of sensitivity of a patient for a given treatment, that can be calculated from the activity of said treatment across a representative population of patients, cannot be used to compare different treatments for the same patient.

The different toxicity for different treatments, and even for different doses approved for the same treatment, or for younger fit vs. older fragile patients, makes the comparison of different treatments even more challenging.

The difficulty in comparing treatments is especially true because the absolute values of their activity (e.g. AUCs) are not normalized to compare drug to drug, treatment to treatment. The novel method proposed here to normalize the AUCs based on population historical records of activity, enables a direct comparison between different treatments in terms of their sensitivity for an individual patient. In a sample of the individual patient, the relative sensitivity of the patient sample for every treatment can be estimated quantitatively using an activity score (e.g. AUC), normalized across a population of similar patients. FIG. 19 shows a ranking obtained using this particular approach to classification for different types of patients.

In one embodiment, each of said systems of the present invention further comprises means for prescribing a plan of care to a subject, wherein said plan of care prescribes said combination of drugs when said disease is determined to be sensitive to treatment with said combination of drugs in said subject. for whom said combination of drugs is determined to be effective in the treatment of said disease in said subject. Correspondingly, each of said method and system of the present invention for determining the efficacy of treatment with a combination of drugs comprising a drug A and a drug B in a subject diagnosed with a disease may further comprise means for prescribing a plan of care to a subject, wherein said plan of care prescribes said combination of drugs when said disease is determined to be sensitive to treatment with said combination of drugs in said subject (i.e. said plan of care to is prescribed to said subject for whom said combination of drugs is determined to be effective in the treatment of said disease in said subject). Thus, the present invention also relates to use of said method or system in prescribing said plan of care to said subject.

Analogously, the method and system of the present invention for classifying the utility of drug combinations each comprising a drug A and a drug B in treatment of a subject diagnosed with a disease may further comprise prescribing a plan of care to said subject, wherein said plan of care prescribes a combination of drugs selected from the combinations of drugs which are classified highest in utility for the treatment of said disease in said subject. Thus, the present invention also relates to use of said method or system in prescribing said plan of care to said subject.

Moreover, the present invention relates to a method of treatment of a subject diagnosed with a disease, comprising administration of a combination of drugs to said subject, when said disease is determined to be sensitive to treatment with said combination of drugs in said subject according to the method or the system for determining the efficacy of treatment with a combination of drugs comprising a drug A and a drug B in a subject diagnosed with a disease.

Analogously, the present invention relates to a method of treatment of a subject diagnosed with a disease, comprising administration of a combination of drugs selected from the combinations of drugs which are classified highest in utility for the treatment of said disease in said subject according to the method or system for classifying the utility of drug combinations each comprising a drug A and a drug B in treatment of a subject diagnosed with a disease.

The present invention also relates to use of the method or the system for determining the efficacy of treatment with a combination of drugs comprising a drug A and a drug B in a subject diagnosed with a disease, for determining whether a given subject from a population of subjects each diagnosed with a disease is suitable for inclusion in a clinical trial involving treatment with a combination of drugs comprising a drug A and a drug B, wherein:
when the disease is determined to be sensitive to treatment with said combination of drugs in said subject, said subject is selected for inclusion in said clinical trial; and
when the disease is determined to be resistant to treatment with said combination of drugs in said subject, said subject is not selected for inclusion in said clinical trial.

Analogously, the present invention relates to use of the method or the system for classifying the utility of drug combinations each comprising a drug A and a drug B in treatment of a subject diagnosed with a disease, for determining whether a given subject from a population of subjects each diagnosed with a disease is suitable for inclusion in a clinical trial involving treatment with a combinations of drugs comprising a drug A and a drug B, wherein:
when said combinations of drugs is classified highest in utility in treatment of said disease in said subject, said subject is selected for inclusion in said clinical trial; and
when said combinations of drugs is classified highest in utility in treatment of said disease in said subject, said subject is not selected for inclusion in said clinical trial. Such uses of the methods and systems of the present invention determine whether a given subject from a population of subjects each diagnosed with a disease is suitable for inclusion in a clinical trial involving treatment with a combination of drugs comprising a drug A and a drug B. These find particular utility when the drugs or the combination thereof are not yet approved by a regulatory authority but require clinical trials to achieve approval. Therefore, the drugs that are selected for inclusion in said clinical trial according to said methods are companion diagnostics (CDx), and are a type of biomarker for that drug treatment. Said companion diagnostic is used to select patients that can be treated with said treatment, which in a clinical trial means a selection criterion; only patients that the CDX identify as sensitive to the treatment can be included in the trial.

### EXAMPLES

The following examples illustrate the invention and should not be considered as limiting, but rather illustrative of the invention.

Material and Methods

(i) Samples

123 monitored newly diagnosed acute myeloid leukemia (AML) patients (de novo or secondary to myelodysplastic syndromes, or therapy-related) aged 18 years and older were used for correlation analysis. All 123 patients received front-line therapy using idarubicin+cytarabine (IDA+CYT) 3+7 (a dosage regime which consists of 7 days of treatment with standard-dose cytarabine, and 3 days of treatment with idarubicin, as specified below). The series used expanded to 473 patients, including all adult patients treated with other front-line regimens, to build pharmacodynamics (PD) population-based models.

Diagnosis and classification of AML was made according to the WHO (World Health Classification) criteria (Vardiman, et al 2002).

(ii) Chemotherapy Regimen, Drugs and Evaluation

Drugs were sourced from Sigma Aldrich, and Selleck Chemicals.

Induction therapy in Examples 1 and 2 consisted of up to two cycles of the combination of intravenous (IV) idarubicin (IDA) (12 mg/m²/day), from days one to three, and IV continuous perfusion of cytarabine (CYT) (200 mg/m²/day), from days one to seven. A second 3+7 induction cycle was administered in patients showing a partial remission (PR) after the first cycle. Patients were considered as responders if they achieved complete remission (CR) or incomplete recovery (CRi) within the first two identical 3+7 induction cycles. Patients dying during induction before response assessment were considered as non-evaluable. The remaining patients were classified as resistant.

(iii) PharmaFlow PM Test

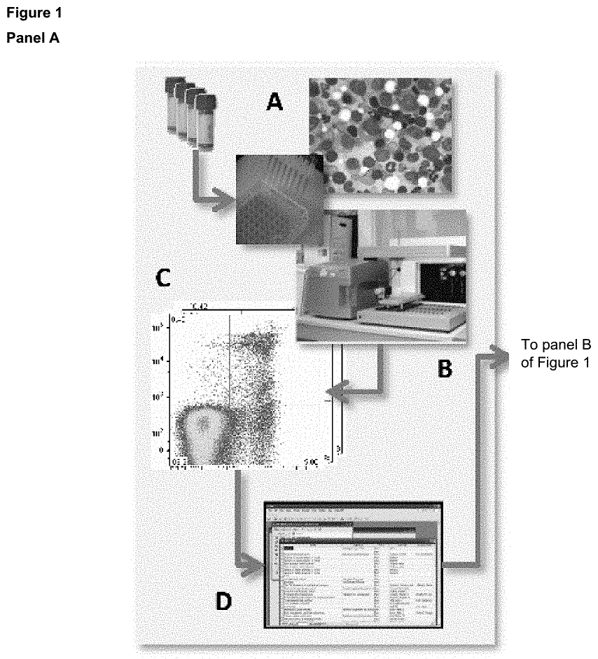

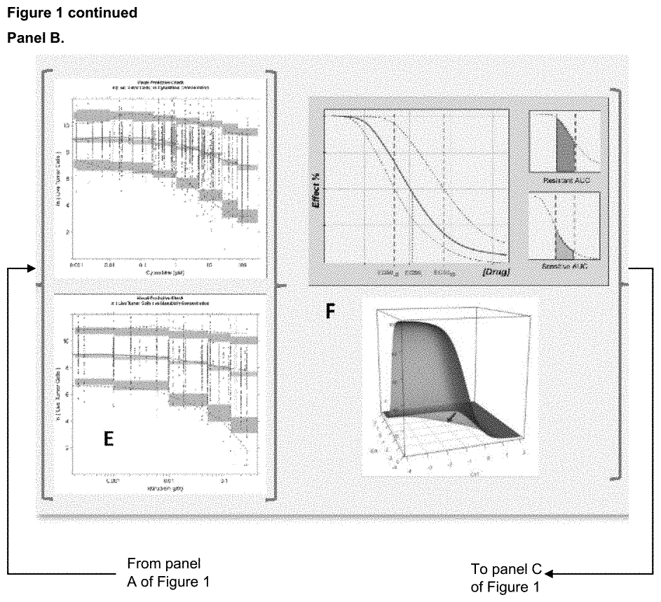

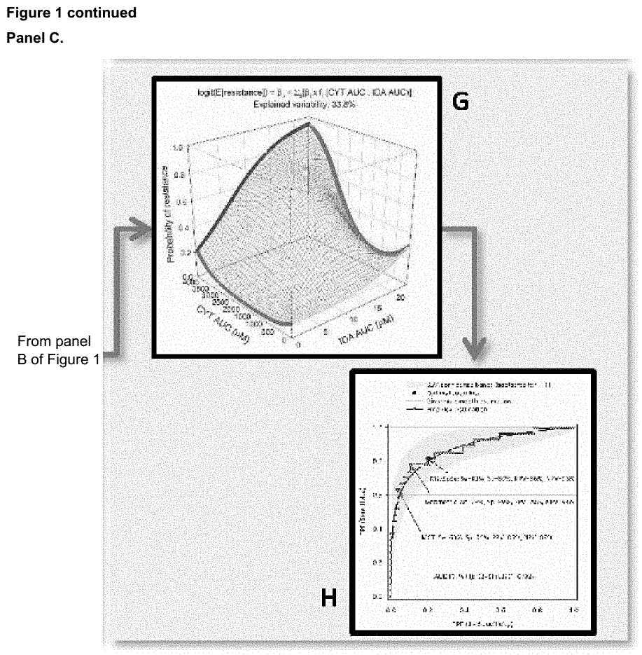

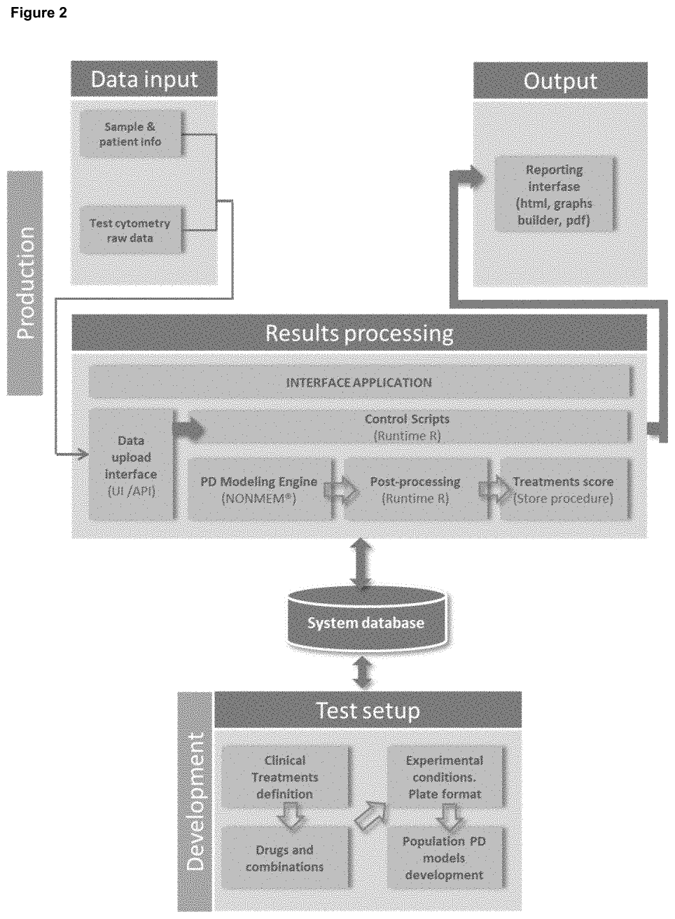

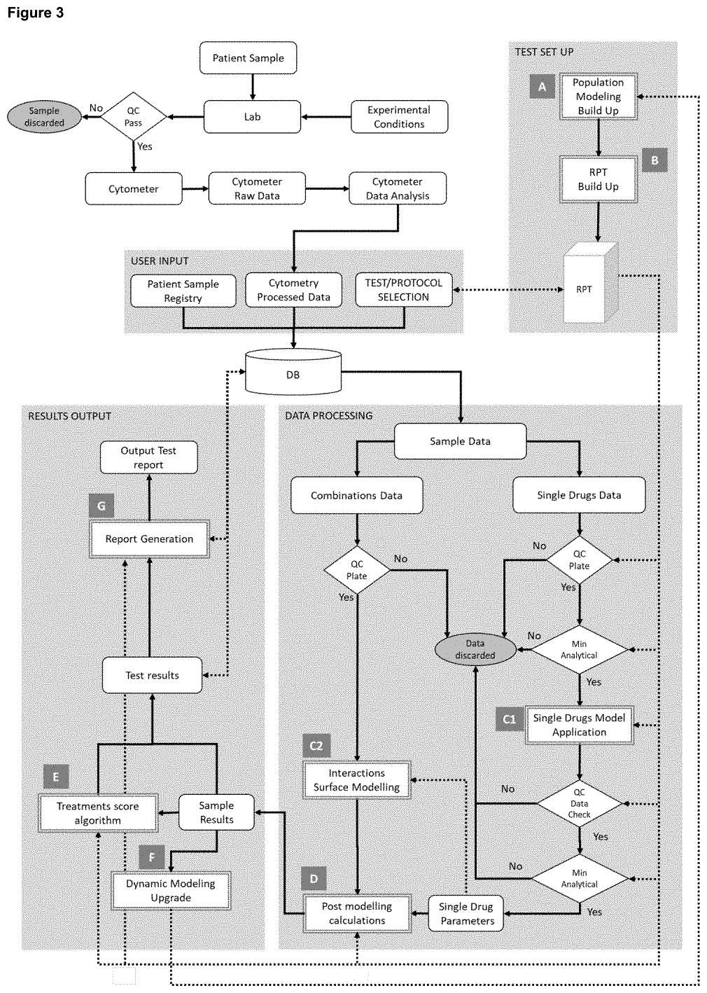

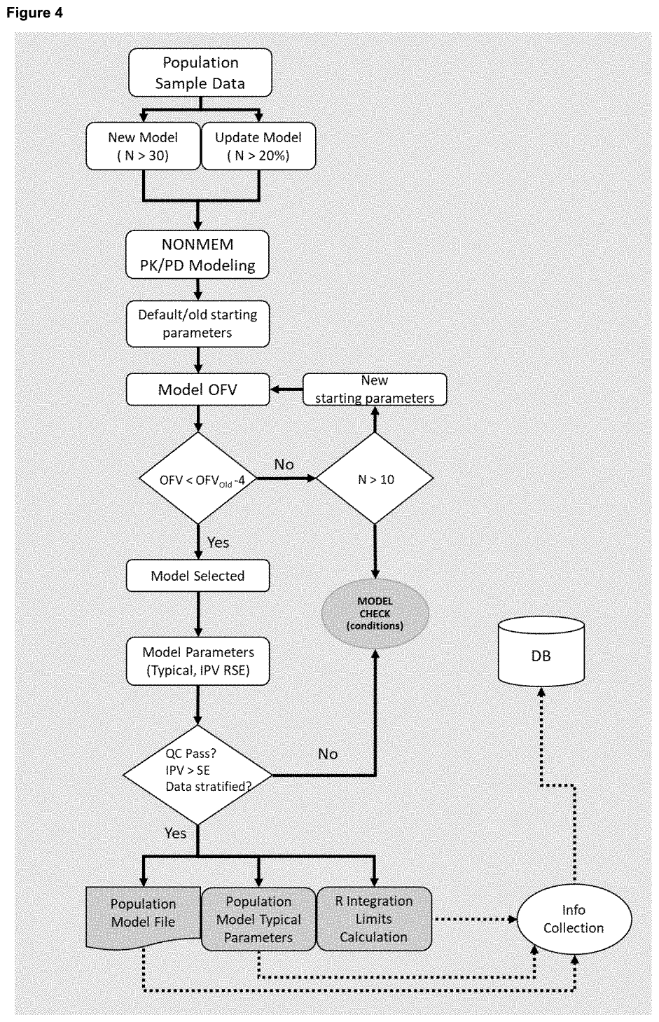

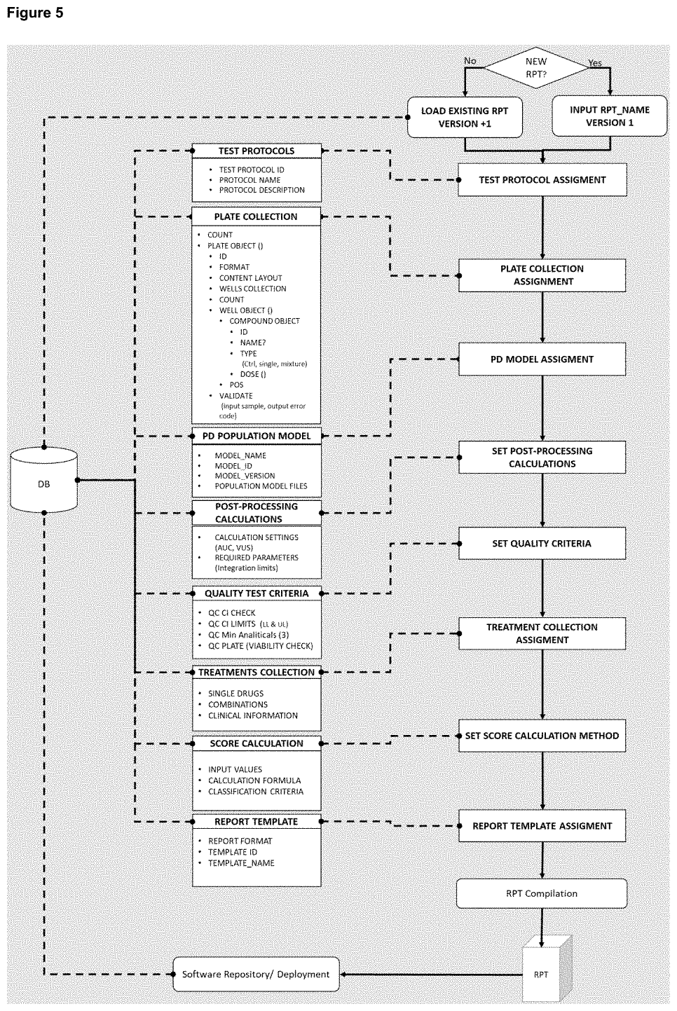

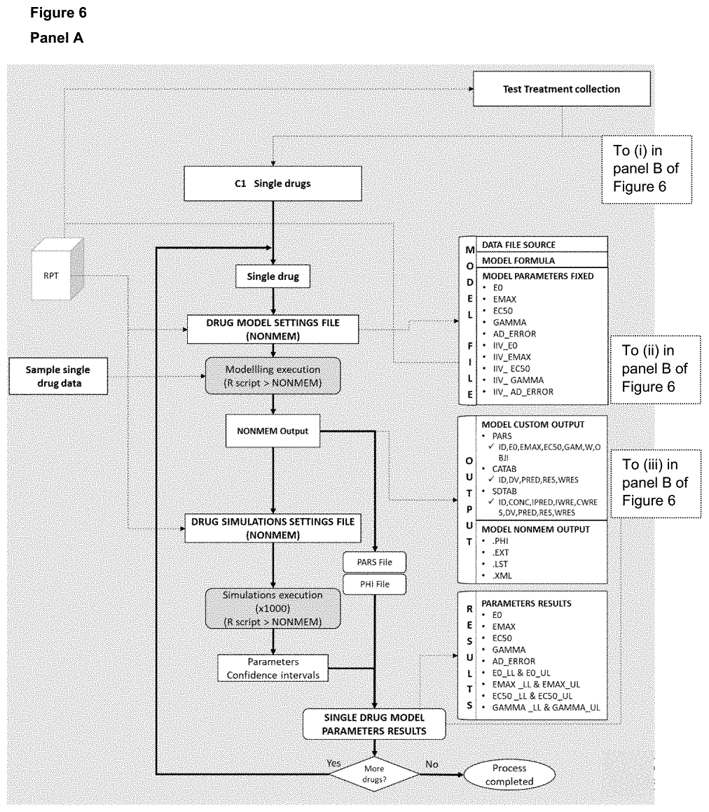

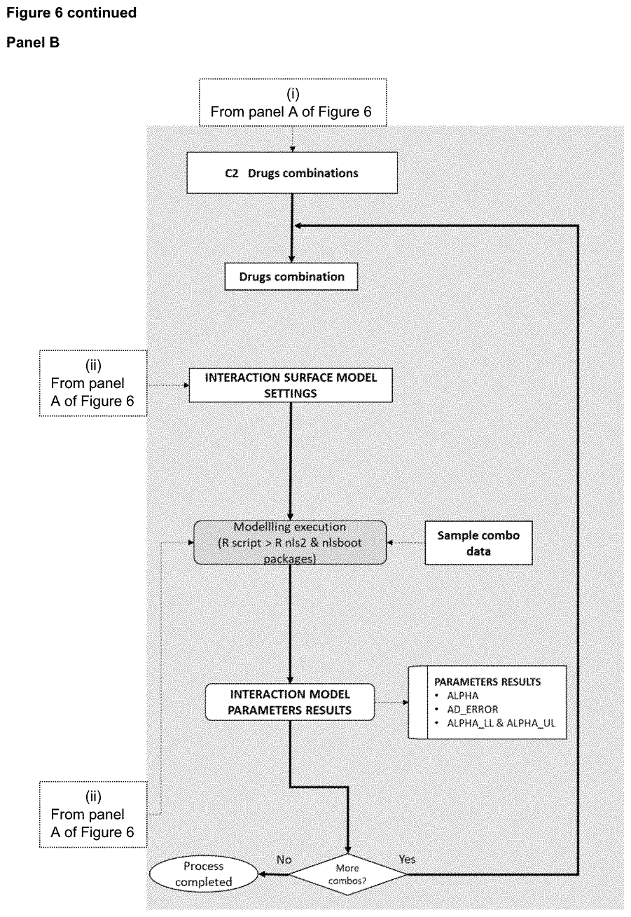

A representative workflow of the PharmaFlow PM test is shown in FIG. 1, collecting the experimental and analytical methods applied in this study.

a) Native Environment Whole Bone Marrow Sample

Ex vivo drug sensitivity analysis was made using the PharmaFlow platform (previously termed ExviTech®) maintaining the bone marrow (BM) microenvironment. A minimum BM sample volume between one and two mL was collected by aspiration at AML diagnosis, before starting induction chemotherapy, and was processed by an automated method 24 hours after extraction. Samples were incubated with CYT, IDA and/or CYT+IDA for 48 hours. A more detailed description of the procedure has been published elsewhere (Bennett, et al 2014).

b) Modeling of Ex Vivo Activity of CYT, IDA, and their Combination

Evaluation of drug response was done by counting the number of live pathological cells (LPC) remaining after incubation at increasing drug concentrations (Upton and Mould 2014). Dying cells (apoptosis) were excluded using Annexin V-FITC. Pharmacological responses were analyzed using PD population-based models (Upton and Mould 2014) which essentially perform the fitting of the dependent variable (natural log of LPC) in a non-linear mixed-effects model to derive typical population values (fixed effects) and the magnitude of inter-patient and residual variability (random effects).

Model development was performed with the first-order conditional estimation method using interaction option with the software NONMEM (v7.2) (Beal, et al 1989-2001), according to the following equation:

$$LPC = {LPC}_{0} \times ( 1 - {E}_{max} \times \frac{{C}^{γ}}{{C}^{γ} + {EC}_{50}^{γ}} )$$

Where LPC₀ (LCTi₀) parameter refers to the number of LPC (LCTi) after incubation in the absence of drug, Emax represents the maximum fractional decrease in LPC that the drug can elicit, EC₅₀, is the drug concentration exerting half of Eₘₐₓ, and γ is the parameter governing the steepness of the LPC vs drug concentration (C) curve. For interaction analysis a Surface Interaction model (Greco, et al 1995) was used to estimate the degree of synergy, referred as a parameter, between both drugs. Interpatient variability (IPV) associated to all parameters was described by means of an exponential model of the components of variance. An additive error structure was used for the residual variability. Population PD models were built with bone marrow (BM) samples from 473 patients that were incubated with CYT, 456 with IDA and 443 with CYT+IDA. Bayesian estimation methods were then used to retrieve individual patient parameters based on their available exposure-response measurements in conjunction with the PD population parameters.

Evaluation of the population PD models was done by the simulation-based procedure visual predictive check (Bergstrand, et al 2011). Five hundred experimental scenarios equal to the original ones were simulated using the selected models and the corresponding parameters. In each simulated set and for each concentration lever the 2.5, 50, and 97.5th percentiles of the LPC distribution were calculated, then the 95% confidence intervals for the above-mentioned percentiles were computed and represented graphically together with the 2.5, 50, and 97.5th percentiles obtained from the raw data.

c) Probability of Clinical Outcome Modeling Clinical Correlation

Individual response profiles normalized with respect to LPC₀ (LCTi₀), were integrated between the concentration points corresponding to the 20th and 80th percentiles of the distribution of estimated individual EC₅₀ values, to obtain the values of the areas under the curves (AUCs) that were used as a descriptor of the ex vivo drug effect (i.e. the higher the AUC, the lower the cytotoxic effect (efficacy or potency) of the drug.

The individual AUC values were correlated to the actual patient's response after induction therapy (non-responder [PR or resistant disease] vs. responder [complete remission (CR) or complete remission with incomplete recovery (CRi)]). The probability of being non-responder was modeled using binary logistic generalized additive models (GAM) based on the binomial distribution that included either one bi- or two univariate smooth functions of the AUCs of CYT and IDA. Additionally, univariate smooth functions of the LPC₀, α, and the pre-post incubation difference of the percentage of LPC in control wells (to detect for any possible effect of spontaneous cell death) were included as well but discarded afterwards because they were not related to the clinical response. Also, the predictive ability of relevant patients' characteristics (age and sex, presenting leukocyte count, performance status, mutations in the NPM1 or FLT3 genes, and cytogenetic risk group) on top of pharmacodynamics data was explored by introducing them as parametric model terms in auxiliary models. P-spline bases were used as smoothers for univariate smooth functions; tensor products of univariate P-spline smooths were used for constructing bi-variate smooth functions. All smoothing bases had dimension three. Coefficients of the smooth functions were estimated using penalized iteratively re-weighted least squares. Minima of the scaled Akaike information criteria were used to find the optimal values of the smoothing parameters.

Additional pharmacodynamic parameters besides the AUCs, for a total of 69 listed in Table 1, were evaluated one by one by GAM models calculating their clinical prediction value. Best parameters were the AUCs of CYT and IDA, and were used to achieve the best correlation function as described above.

TABLE 1
Pharmacodynamic parameters analyzed

| Pharmacodynamic | |
|---|---|
| parameter | Brief description |
| CYT_E0_FREE | Initial Live Pathological Cells number using a free model fitting for |
| | CYT |
| CYT_EMAX_FREE | E_{MAX} value using a free model fitting for CYT |
| CYT_EC50_FREE | EC₅₀ value using a free model fitting for CYT |
| CYT_GAMMA_FREE | γ value using a free model fitting for CYT |
| CYT_W_FREE | Free model Goodness fitting value for CYT |
| IDA_E0_FREE | Initial Live Pathological Cells number using a free model fitting for |
| | IDA |
| IDA_EMAX_FREE | E_{MAX} value using a free model fitting for IDA |
| IDA_EC50_FREE | EC₅₀ value using a free model fitting for IDA |
| IDA_GAMMA_FREE | γ value using a free model fitting for IDA |
| IDA_W_FREE | Free model Goodness fitting value for IDA |
| CYT_E0_FIXED | Initial Live Pathological Cells number using a fixed E_{MAX} model |
| | fitting for CYT |
| CYT_EMAX_FIXED | E_{MAX} value using a fixed E_{MAX} model fitting for CYT |
| CYT_EC50_FIXED | EC₅₀ value using a fixed E_{MAX} model fitting for CYT |
| CYT_GAMMA_FIXED | γ value using a fixed E_{MAX} model fitting for CYT |
| CYT_W_FIXED | Fixed E_{MAX} model Goodness fitting value for CYT |
| IDA_E0_FIXED | Initial Live Pathological Cells number using a fixed E_{MAX} model |
| | fitting for IDA |
| IDA_EMAX_FIXED | E_{MAX} value using a fixed E_{MAX} model fitting for IDA |
| IDA_EC50_FIXED | EC₅₀ value using a fixed E_{MAX} model fitting for IDA |
| IDA_GAMMA_FIXED | γ value using a fixed E_{MAX} model fitting for IDA |
| IDA_W_FIXED | Fixed E_{MAX} model Goodness fitting value for IDA |
| E0_COMB_FREE | Initial Live Pathological Cells number using a free interaction |
| | model fitting |
| CYT_EMAX_COMB_FREE | E_{MAX} value using a free interaction model fitting for CYT |
| CYT_EC50_COMB_FREE | EC₅₀ value using a free interaction model fitting for CYT |
| CYT_GAMMA_COMB_FREE | γ value using a free interaction model fitting for CYT |
| CYT_W_COMB_FREE | Free interaction model Goodness fitting value for CYT |
| IDA_EMAX_COMB_FREE | E_{MAX} value using a free interaction model fitting for IDA |
| IDA_EC50_COMB_FREE | EC₅₀ value using a free interaction model fitting for IDA |
| IDA_GAMMA_COMB_FREE | γ value using a free interaction model fitting for IDA |
| IDA_W_COMB_FREE | Free interaction model Goodness fitting value for IDA |
| EO_COMB_FIXED | Initial Live Pathological Cells number using a fixed E_{MAX} |
| | interaction model fitting |
| CYT_EMAX_COMB_FIXED | EMAX value using a fixed E_{MAX} interaction model fitting for CYT |
| CYT_EC50_COMB_FIXED | EC₅₀ value using a fixed E_{MAX} interaction model fitting for CYT |
| CYT_GAMMA_COMB_FIXED | γ value using a fixed E_{MAX} interaction model fitting for CYT |
| CYT_W_COMB_FIXED | Fixed E_{MAX} interaction model Goodness fitting value for CYT |
| IDA_EMAX_COMB_FIXED | E_{MAX} value using a fixed E_{MAX} interaction model fitting for IDA |
| IDA_EC50_COMB_FIXED | EC₅₀ value using a fixed EMAX interaction model fitting for IDA |
| IDA_GAMMA_COMB_FIXED | γ value using a fixed EMAX interaction model fitting for IDA |
| IDA_W_COMB_FIXED | Fixed E_{MAX} interaction model Goodness fitting value for IDA |
| COMBINATION INDEX | Combination Index value (Sinergy) |
| ALPHA_FREE | Alpha Value (E_{MAX} free Model Interaction parameter) |
| ALPHA_FIXED | Alpha Value (Fixed Model Interaction parameter) |
| CYT_AUC_FREE_DOSES | Area Under the Curve value from free model for CYT |
| CYT_AUC_FIXED_DOSES | Area Under the Curve value from fixed model for CYT |
| IDA_AUC_FREE_DOSES | Area Under the Curve value from free model for IDA |
| IDA_AUC_FIXED_DOSES | Area Under the Curve value from fixed model for IDA |
| VUS_FREE_DOSES | Volume Under the Surface value from free model |
| VUS_FIXED_DOSES | Volume Under the Surface value from fixed model |
| CYT_AUC_FREE_LIMITS | CYT Area Under the Curve value from free model using specific |
| | limits |
| CYT_AUC_FIXED_LIMITS | CYT Area Under the Curve value from fixed model using specific |
| | limits |
| IDA_AUC_FREE_LIMITS | IDA Area Under the Curve value from free model using specific |
| | limits |
| IDA_AUC_FIXED_LIMITS | IDA Area Under the Curve value from fixed model using specific |
| | limits |
| VUS_FREE_LIMITS | Volume Under the Surface value from free model using specific |
| | limits |
| VUS_FIXED_LIMITS | Volume Under the Surface value from fixed model using specific |
| | limits |
| CYT_AUC_20_80 | CYT Area Under the Curve value from fixed model using |
| | definitive limits on X axis |
| CYT_AUC_XY | CYT Area Under the Curve value from fixed model using |
| | definitive limits on X axis & Y axis |
| CYT_AUC_PERCENT | CYT Normalized Area Under the Curve on fixed model using |
| | definitive limits on X axis & Y axis |
| IDA_AUC_20_80 | IDA Area Under the Curve value from fixed model using definitive |
| | limits on X axis |
| IDA_AUC_XY | IDA Area Under the Curve value from fixed model using definitive |
| | limits on X axis & Y axis |
| IDA_AUC_PERCENT | IDA Normalized Area Under the Curve on fixed model using |
| | definitive limits on X axis & Y axis |
| CYT_AUC_PERCENT_DIF | CYT Normalized Area Under the Curve on fixed model using |
| | definitive limits on X axis & Y axis substration (Upper minus lower |
| | limit) |
| IDA_AUC_PERCENT_DIF | IDA Normalized Area Under the Curve on fixed model using |
| | definitive limits on X axis & Y axis substration (Upper minus lower |
| | limit) |
| QC_PASS_AUC | Both AUC_PERCENT_DIF should be under 40. |
| VUS_PERCENT | Normalized Volume Under the Surface on fixed model using |
| | definitive limits on X axis & Y axis |
| VIABILITY_T0 | Number of LPC at Time = 0 h |
| VIABILITY_POST_INC | Number of LPC at post-incubation time |
| VIABILITY_DIF | VIABILITY_POST_INC - VIABILITY_T0 |
| VIABILITY_v2 | VIABILITY_DIF/VIABILITY_POST_INC |
| SCORE_V1 | Score result from different algorithms |
| QC_PASS_VIAB | VIABILITY_DIF should be less or equal to 60 |

(iv) Data Collection and Study Endpoints

Demographic data (gender, age) were prospectively collected since diagnosis, as well as the following parameters: count of mononuclear cells obtained from peripheral blood (WBC in PB), Eastern Cooperative Oncology Group (ECOG) Performance Status, type of AML (French-American-British (FAB) classification, de novo vs. secondary AML), karyotype (Grimwade, et al. 2010), FLT3 and NPM1 mutation status, hematological response, number of 3+7 induction cycles, date of response, date of last follow-up, and post-remission therapy. All data collection forms and clinical records were monitored.

The primary end-point was evaluating the predictive capacity of the ex vivo results. First, the CR/CRi rate observed in patients treated with up to two induction cycles of 3+7 was recorded and monitored. This was correlated with the ex vivo drug sensitivity analyses performed in the same cohort of patients. As a secondary end-point, the overall survival (OS) probability was also calculated according to the observed and predicted response after induction.

Since the prediction of outcome to front-line induction therapy is likely to be most beneficial to elderly patients, sensitivity analyses of the clinical correlation were performed by re-running the GAMs within the cohort aged 60 years or more (n=31).

(v) Statistical Analyses

The probability of response modeling was performed with the mgcv package (v1.8-23) run in the R environment (v3.4.3) for statistical computing (Wood 2006). The empirical receiver operating characteristic (ROC) curves were calculated for the probabilities of being non-responder from each GAM. The AUCs of ROC curves were computed using the trapezoidal rule. In addition, three cut-points to define positivity and derive classification probabilities (sensitivity and specificity) were established for each ROC curve. One used a geometric criterion, by selecting the closest point to the (1,0) coordinate (left upper corner of the [sensitivity,1-specificity] plane), another was set by maximizing both sensitivity and specificity and the other by minimizing a misclassification cost term (Greiner 1996), assigning a greater cost to false positives than to false negatives (prioritizing specificity over sensitivity).

The OS was described with the Kaplan-Meier method and compared between the patients predicted to be non-responder and responder as per the aforementioned three different cut-points using simple Cox regression.

### Example 1. Prediction of Clinical Response after Idarubicin and Cytarabine Induction Therapy

The following present a method to determine the correlation between the observed CR/CRi rate after idarubicin (IDA) and cytarabine (CYT) 3+7 induction and the leukemic chemosensitivity measured by an ex vivo test of drug activity. Bone marrow samples from adult patients with newly diagnosed AML were included in this study. Whole bone marrow samples were incubated for 48 h in well plates containing IDA, CYT, or their combination. Pharmacological response parameters were estimated using population pharmacodynamic models. Patients attaining a CR/CRi with up to two induction cycles of 3+7 were classified as responders and the remaining as resistant. A total of 123 patients fulfilled the inclusion criteria and were evaluable for correlation analyses. The strongest clinical predictors were the area under the curve of the concentration response curves of CYT and IDA. The overall accuracy achieved using MaxSpSe criteria to define positivity was 81%, predicting better responder (93%) than non-responder patients (60%). The ex vivo test provides better yet similar information than cytogenetics but can be provided before treatment representing a valuable in-time addition.

Results

(vi) Patient Characteristics

Overall, 954 BM samples from patients with AML suspicion were received at the laboratory. Of them, 316 (33%) were not evaluable because of the following laboratory technical issues:

1) low sample cellularity (187 patients),

2) low cell viability (below 60%) in control wells after incubation (67),

3) insufficient sample volume (<500 μL) (38), and

4) other reasons such as clotted sample (24). Other 26 patients (3%) did not fulfill the diagnosis criteria. Among the 612 analyzed samples, 139 where used only for assay adjustment and did not contain necessary data for the final model. Overall, 473 patient samples (50%) were used to build the PD models, and a complete data set was monitored in 237 of them (50%). Among the monitored patients, 114 were not evaluable for the correlation analyses due to: 1) induction death (20 patients), 2) not first line of treatment (11), and 3) other induction schedule (83). Finally, 123 monitored patients (52%) fulfilled the inclusion criteria defined in the study and were evaluable for the correlation analyses. The main patient and disease characteristics of these 123 patients are displayed in Table 2. In summary, median age was 50 years (range, 19 to 71), 109 patients (89%) were diagnosed with de novo AML, and 21 patients (17%) were categorized as having high-risk cytogenetics. Only the cytogenetic risk group and, marginally, the presence of mutations in the NPM1 gene were significantly associated with clinical response to induction and the result of the PM test. Post-remission therapy consisted of allogeneic stem cell transplant (SCT) in 33 patients (27%), and chemotherapy with or without autologous SCT in 66 patients (54%).

TABLE 2
Characteristics of patients

| (A) clinical responses and geometric criteria. | | |
|---|---|---|
| | Clinical response (ref. standard) | Geometric criterion |

| | | | | | |
|---|---|---|---|---|---|
| CR/CRi | PR/Res | | Sensitive | Resistant | |
| (N = 92) | (N = 31) | p-value | (N = 82) | (N = 41) | p-value |

Age (years)

| | | | | | | | | | | |
|---|---|---|---|---|---|---|---|---|---|---|
| Median (range) | 49 | (19-71) | 55 | (22-67) | 0.224 ^{a} | 49 | (19-70) | 54 | (20-71) | 0.170 ^{a} |
| 18-29 [n (%)] | 8 | (8.7) | 2 | (6.5) | 0.758 ^{b} | 7 | (8.5) | 3 | (7.3) | 0.675 ^{b} |
| 30-39 [n (%)] | 17 | (18.5) | 5 | (16.1) | | 17 | (20.7) | 5 | (12.2) | |
| 40-49 [n (%)] | 23 | (25.0) | 5 | (16.1) | | 18 | (22.0) | 10 | (24.4) | |
| 50-59 [n (%)] | 22 | (23.9) | 10 | (32.3) | | 22 | (26.8) | 10 | (24.4) | |
| >60 | 22 | (23.9) | 9 | (29.0) | | 18 | (22.0) | 13 | (31.7) | |

Gender

| | | | | | | | | | | |
|---|---|---|---|---|---|---|---|---|---|---|
| Male [n (%)] | 42 | (45.7) | 16 | (51.6) | 0.565 ^{b} | 37 | (45.1) | 21 | (51.2) | 0.523 ^{b} |
| Female [n (%)] | 50 | (54.3) | 15 | (48.4) | | | | | | |

ECOG

| | | | | | | | | | | |
|---|---|---|---|---|---|---|---|---|---|---|
| 0 [n (%)] | 39 | (49.4) | 9 | (33.3) | 0.042 ^{b} | 33 | (44.6) | 15 | (46.9) | 0.496 ^{b} |
| 1 [n (%)] | 30 | (38.0) | 18 | (66.7) | | 32 | (43.2) | 16 | (50.0) | |
| 2 [n (%)] | 7 | (8.9) | 0 | (0.0) | | 6 | (8.1) | 1 | (3.1) | |
| 3-4 [n (%)] | 3 | (3.8) | 0 | (0.0) | | 3 | (4.1) | 0 | (0.0) | |

FAB subtype

| | | | | | | | | | | |
|---|---|---|---|---|---|---|---|---|---|---|
| M0 [n (%)] | 3 | (3.6) | 5 | (17.9) | 0.166 ^{b} | 3 | (3.9) | 5 | (13.9) | 0.358 ^{b} |
| M1 [n (%)] | 19 | (22.6) | 4 | (14.3) | | 15 | (19.7) | 8 | (22.2) | |
| M2 [n (%)] | 23 | (27.4) | 9 | (32.1) | | 24 | (31.6) | 8 | (22.2) | |
| M4 [n (%)] | 22 | (26.2) | 5 | (17.9) | | 20 | (26.3) | 7 | (19.4) | |
| M5 [n (%)] | 16 | (19.0) | 5 | (17.9) | | 13 | (17.1) | 8 | (22.2) | |
| M6 [n (%)] | 1 | (1.2) | 0 | (0.0) | | 1 | (1.3) | 0 | (0.0) | |

WBC (count × 10⁹L⁻¹)

| | | | | | | | | | | |
|---|---|---|---|---|---|---|---|---|---|---|
| Median (range) | 22.1 | (0-288.4) | 22.3 | (1-157) | 0.461 ^{a} | 20.6 | (0-288.4) | 22.4 | (1-157) | 0.359 ^{a} |
| 0-10 [n (%)] | 31 | (33.7) | 12 | (38.7) | 0.528 ^{b} | 28 | (34.1) | 15 | (36.6) | 0.231 ^{b} |
| 10-50 [n (%)] | 37 | (40.2) | 14 | (45.2) | | 31 | (37.8) | 20 | (48.8) | |
| >50 [n (%)] | 24 | (26.1) | 5 | (16.1) | | 23 | (28.0) | 6 | (14.6) | |

Cytogenetic risk prof.

| | | | | | | | | | | |
|---|---|---|---|---|---|---|---|---|---|---|
| Favorable [n (%)] | 11 | (13.1) | 0 | (0.0) | <0.001 ^{b} | 10 | (13.3) | 1 | (2.8) | <0.001 ^{b} |
| Intermediate [n (%)] | 68 | (81.0) | 11 | (40.7) | | 60 | (80.0) | 19 | (52.8) | |
| Adverse [n (%)] | 5 | (6.0) | 16 | (59.3) | | 5 | (6.7) | 16 | (44.45) | |

FLT3-ITD status

| | | | | | | | | | | |
|---|---|---|---|---|---|---|---|---|---|---|
| Wild type [n (%)] | 72 | (79.1) | 28 | (90.3) | 0.161 ^{b} | 64 | (79.0) | 36 | (87.8) | 0.233^{b} |
| Mutant [n (%)] | 19 | (20.9) | 3 | (9.7) | | 17 | (21.0) | 5 | (12.2) | |

NPM1 status

| | | | | | | | | | | |
|---|---|---|---|---|---|---|---|---|---|---|
| Wild type [n (%)] | 47 | (57.3) | 22 | (78.6) | 0.045 ^{b} | 42 | (57.5) | 27 | (73.0) | 0.114^{b} |
| Mutant [n (%)] | 35 | (42.7) | 6 | (21.4) | | 31 | (42.5) | 10 | (27.0) | |

(B) Criteria where specificity and sensitivity are maximised and where specificity is prioritized over sensitivity.

| MaxSpSe criterion | | | mMCT criterion | | |
|---|---|---|---|---|---|
| Sensitive | Resistant | | Sensitive | Resistant | |
| (N = 81) | (N = 42) | p-value | (N = 99) | (N = 24) | p-value |

Age (years)

| | | | | | | | | | | |
|---|---|---|---|---|---|---|---|---|---|---|
| Median (range) | 49 | (19-70) | 52.5 | (19-71) | 0.298 ^{a} | 49 | (19-71) | 55.5 | (22-67) | 0.315 ^{a} |
| 18-29 [n (%)] | 6 | (7.4) | 4 | (9.5) | 0.660 ^{b} | 8 | (8.1) | 2 | (8.3) | 0.938 ^{b} |
| 30-39 [n (%)] | 17 | (21.0) | 5 | (11.9) | | 18 | (18.2) | 4 | (16.7) | |
| 40-49 [n (%)] | 18 | (22.2) | 10 | (23.8) | | 24 | (24.2) | 4 | (16.7) | |
| 50-59 [n (%)] | 22 | (27.2) | 10 | (23.8) | | 25 | (25.3) | 7 | (29.2) | |
| >60 | 18 | (22.2) | 13 | (31.0) | | 24 | (24.2) | 7 | (29.2) | |

Gender

| | | | | | | | | | | |
|---|---|---|---|---|---|---|---|---|---|---|
| Male [n (%)] | 37 | (45.7) | 21 | (50.0) | 0.649 ^{b} | 46 | (46.5) | 12 | (50.0) | 0.756 ^{b} |
| Female [n (%)] | 44 | (54.3) | 21 | (50.0) | | | | | | |

ECOG

| | | | | | | | | | | |
|---|---|---|---|---|---|---|---|---|---|---|
| 0 [n (%)] | 33 | (45.2) | 15 | (45.5) | 0.441 ^{b} | 40 | (46.5) | 8 | (40.0) | 0.301 ^{b} |
| 1 [n (%)] | 31 | (42.5) | 17 | (51.5) | | 36 | (41.9) | 12 | (60.0) | |
| 2 [n (%)] | 6 | (8.2) | 1 | (3.0) | | 7 | (8.1) | 0 | (0.0) | |
| 3-4 [n (%)] | 3 | (4.1) | 0 | (0.0) | | 3 | (3.5) | 0 | (0.0) | |

FAB subtype

| | | | | | | | | | | |
|---|---|---|---|---|---|---|---|---|---|---|
| M0 [n (%)] | 3 | (4.0) | 5 | (13.5) | 0.418 ^{b} | 7 | (7.8) | 1 | (4.5) | 0.935 ^{b} |
| M1 [n (%)] | 15 | (20.0) | 8 | (21.6) | | 18 | (20.0) | 5 | (22.7) | |
| M2 [n (%)] | 24 | (32.0) | 8 | (21.6) | | 25 | (27.8) | 7 | (31.8) | |
| M4 [n (%)] | 19 | (25.3) | 8 | (21.6) | | 23 | (25.6) | 4 | (18.2) | |
| M5 [n (%)] | 13 | (17.3) | 8 | (21.6) | | 16 | (17.8) | 5 | (22.7) | |
| M6 [n (%)] | 1 | (1.3) | 0 | (0.0) | | 1 | (1.1) | 0 | (0.0) | |

WBC (count × 10⁹L⁻¹)

| | | | | | | | | | | |
|---|---|---|---|---|---|---|---|---|---|---|
| Median (range) | 19.7 | (0-288.4) | 22.6 | (1-157) | 0.491 ^{a} | 22.7 | (0-288.4) | 21.9 | (1.3-157) | 0.243 ^{a} |
| 0-10 [n (%)] | 28 | (34.6) | 15 | (35.7) | 0.390 ^{b} | 32 | (32.3) | 11 | (45.8) | 0.422 ^{b} |
| 10-50 [n (%)] | 31 | (38.3) | 20 | (47.6) | | 42 | (42.4) | 9 | (37.5) | |
| >50 [n (%)] | 22 | (27.2) | 7 | (16.7) | | 25 | (25.3) | 4 | (16.7) | |

Cytogenetic risk prof.

| Favorable [n (%)] | 9 | (12.2) | 2 | (5.4) | <0.001 ^{b} | 11 | (12.1) | 0 | (0.0) | <0.001 ^{b} |
|---|---|---|---|---|---|---|---|---|---|---|
| Intermediate [n (%)] | 60 | (81.1) | 19 | (51.4) | | 73 | (80.2) | 6 | (30.0) | |
| Adverse [n (%)] | 5 | (6.8) | 16 | (43.2) | | 7 | (7.7) | 14 | (70.0) | |
| FLT3-ITD status | | | | | | | | | | |
| Wild type [n (%)] | 63 | (78.7) | 37 | (88.1) | 0.202 ^{b} | 78 | (79.6) | 22 | (91.7) | 0.168 ^{b} |
| Mutant [n (%)] | 17 | (21.2) | 5 | (11.9) | | 20 | (20.4) | 2 | (8.3) | |

NPM1 status

| | | | | | | | | | | |
|---|---|---|---|---|---|---|---|---|---|---|
| Wild type [n (%)] | 41 | (56.9) | 28 | (73.7) | 0.084 ^{b} | 52 | (59.8) | 17 | (73.9) | 0.212 ^{b} |
| Mutant [n (%)] | 31 | (43.1) | 10 | (26.3) | | 35 | (40.2) | 6 | (26.1) | |

Patients with missing data have not been included in the denominators of the relative frequencies.
^{a}Mann-Whitney test;
^{b}Pearson's chi-square test

(vii) Ex Vivo PharmaFlow Test Characterization of CYT-IDA Combination

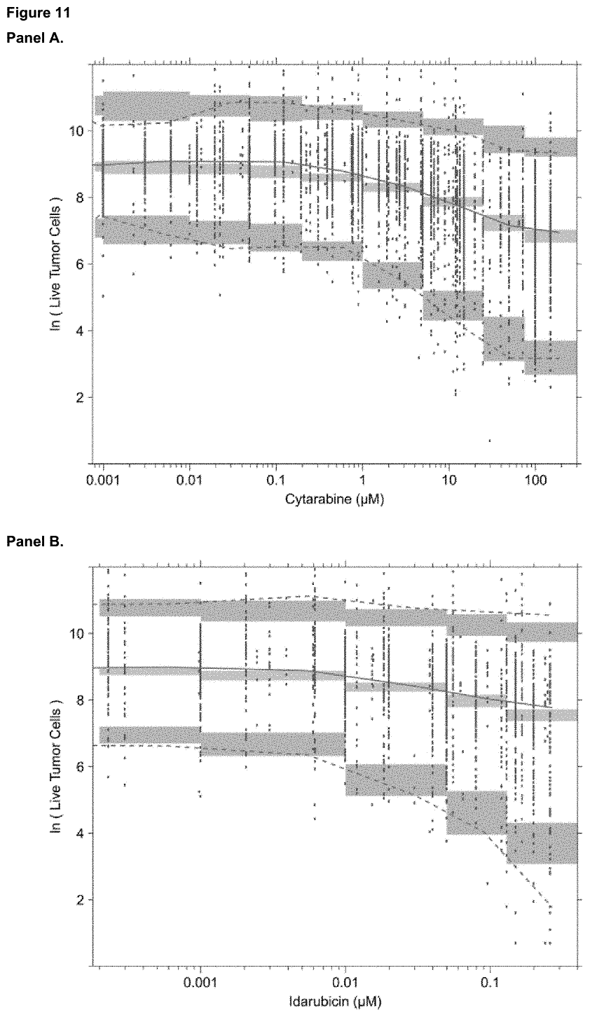

Visual predictive checks graphs were generated for the single drugs PD models (FIG. 11). Most of the observations were contained within the simulation-based 95% confidence intervals of the 2.5-97.5th population percentiles proving good predictability of the selected models. Pharmacodynamic population parameters as well as variability and error values are shown in Table 3. The typical parameter values for maximal fractional effect (Eₘₐₓ) were set to 1 for both drugs and was limited to the range 0-3. The typical value for the alpha parameter of the interaction model was 1.1 (Table 3), indicating slight synergistic interaction between IDA and CYT in the ex vivo combination experiments.

TABLE 3
Estimation of the ex vivo population pharmacodynamic parameters.
Single Drugs

| | | |
|---|---|---|
| Parameter (units) | Cytarabine | Idarubicin |

| | | | | |
|---|---|---|---|---|
| LPC₀ (cells) | 7530 | (4.2) | 7270 | (4.8) |
| E_{MAX} (unitless) | 1 | (—) | 1 | (—) |
| EC₅₀ (μM) | 6.94 | (13.3) | 0.087 | (9.2) |
| γ(unitless) | 0.684 | (—) | 1.14 | (—) |
| Residual Error (log(_{μ}M)) | 0.231 | (2.9) | 0.237 | (3.2) |
| Inter-patient variability (IPV) | | | | |
| LPC₀ | 89.7 | (2.4) | 92.7 | (2.6) |

| | | |
|---|---|---|
| E_{MAX} | N/D | N/D |

| | | | | |
|---|---|---|---|---|
| EC₅₀ | 229 | (4.4) | 159 | (4.5) |

| | | |
|---|---|---|
| γ | N/D | N/D |

| | | | | |
|---|---|---|---|---|
| Residual Error | 50.5 | (3.8) | 45.8 | (4) |

Drug Combination

| Parameter (units) | Cytarabine + Idarubicin | |
|---|---|---|
| α (unitless) | 1.1 | (13) |
| IPV α [CV (%)] | 176 | (4.8) |
| Residual Error [log(_{μ}M)] | 0.299 | (4.2) |
| IPV Residual Error | 58.3 | (4.4) |

Parameters typical and random (variability and residual error percentage) are shown together with the corresponding relative standard error calculated as the ratio between the standard error provided by NONMEM and the estimate. Estimates of inter-patient variability (IPV) are expressed as coefficient of variation (%).

(viii) Clinical Responses Among AML Patients Treated with CYT-IDA

CR/CRi was achieved after one (88, 96%) or two (4, 4%) identical induction cycles in 92 out of 123 patients (75%) included in the correlation study.

(ix) Correlation Between Ex Vivo Activity and Clinical Response to CYT-IDA

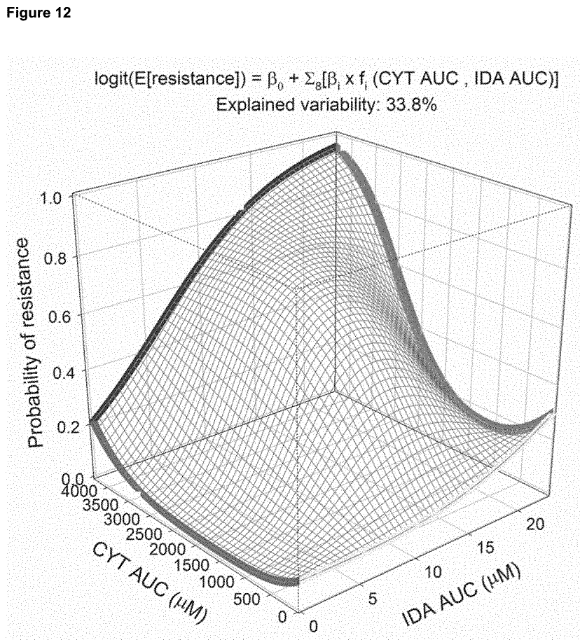

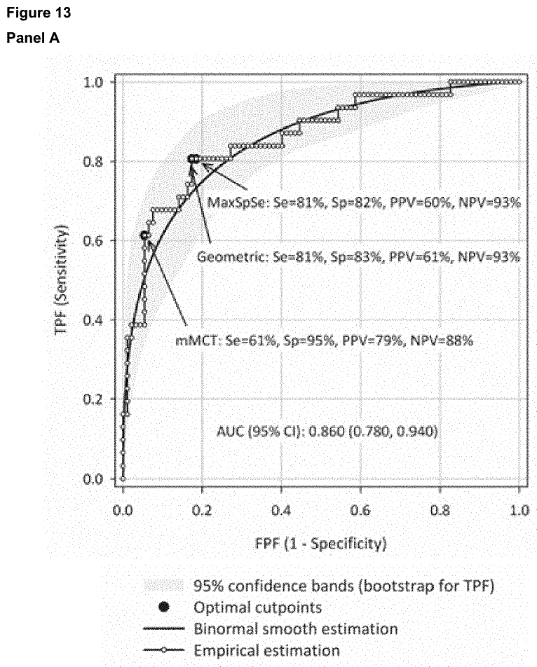

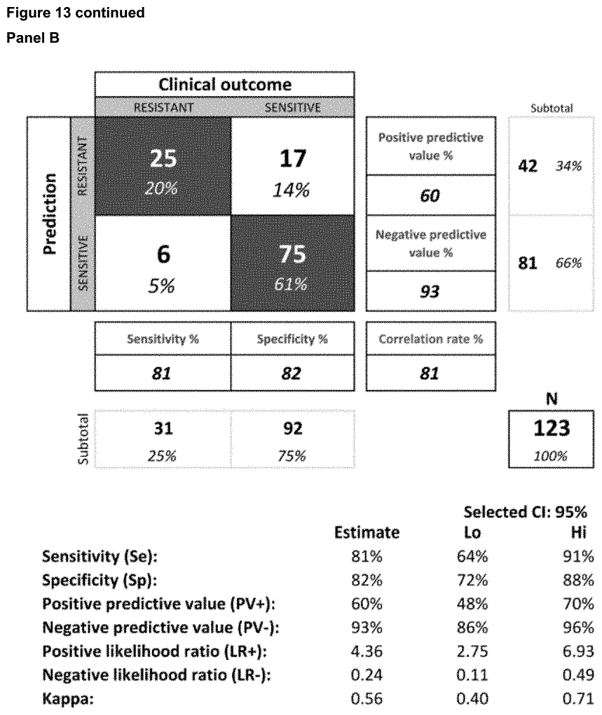

FIG. 12 depicts the predicted surface fitted by the GAM representing the probability of being non-responder for the observed range of the individual AUC values. The model presented used a bivariate smooth function of CYT and IDA; the models that used univariate smooths achieved worse fit. Higher CYT and IDA AUC values were associated with greater probability of being non-responder, albeit the relationship was non-monotonical. Sensitivity/specificity values ranged from 81%/82%, to 61%/95%, based on the cut-off point selected (FIG. 13, panel A). Although the geometric cutoff point balanced both aspects, the final selected cutoff point in ROC curve was the MaxSpSe to construct the confusion matrix and derive classification probabilities, achieving high values for both specificity and sensitivity, as well as good PPV and NPV (FIG. 13, panel B). The positive/negative predictive values (PPV/NPV) ranged from 60%/93% to 79%/88% [i.e. the accuracy achieved using the aforementioned MaxSpSe criteria to define positivity was 81%, predicting better responder (93%, NPV) than non-responder patients (60%, PPV)]. FIG. 13, panel B shows the confusion matrix obtained using the MaxSpSe cutoff point

The sensitivity analyses showed that the predictive ability of the PM test remained intact within the cohort aged ≥60 years, although in this case most of the discriminative information was provided by CYT data; IDA AUC values were in general higher in older patients.

(x) Overall Survival (OS) According to the Ex Vivo Activity and Observed Clinical Response

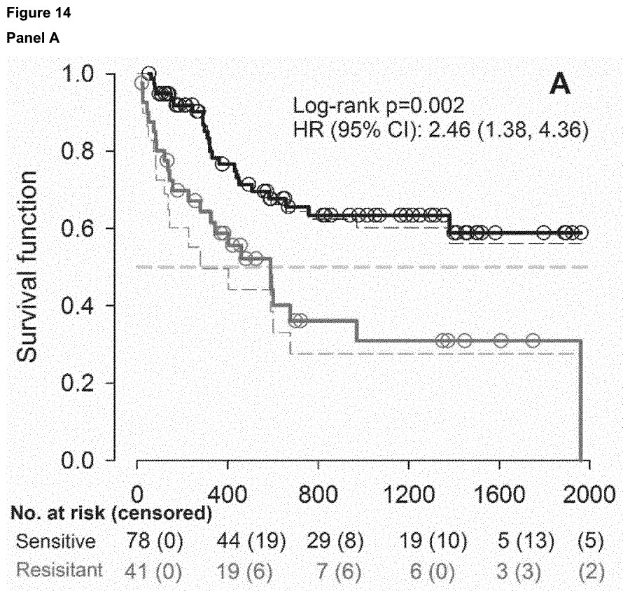

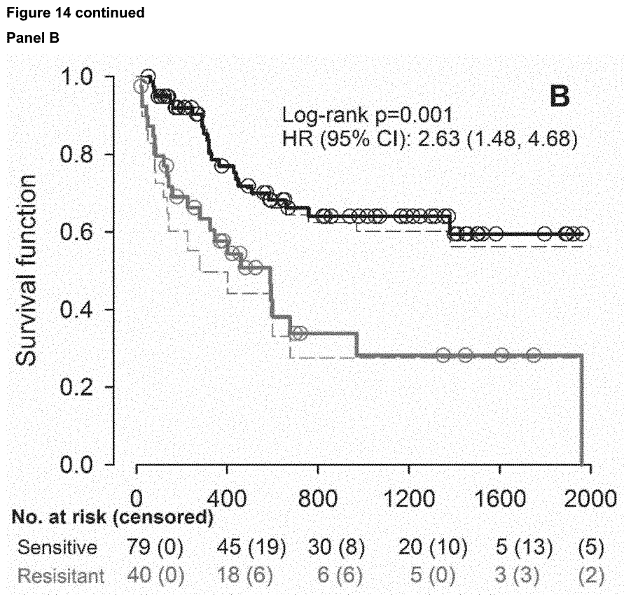

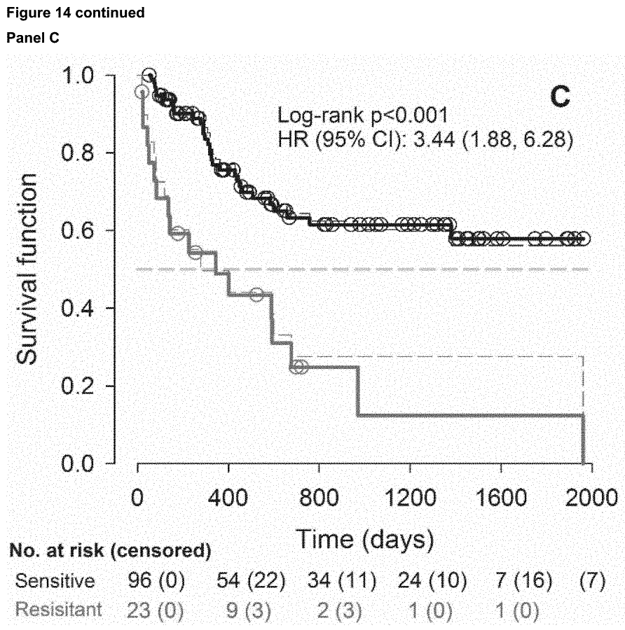

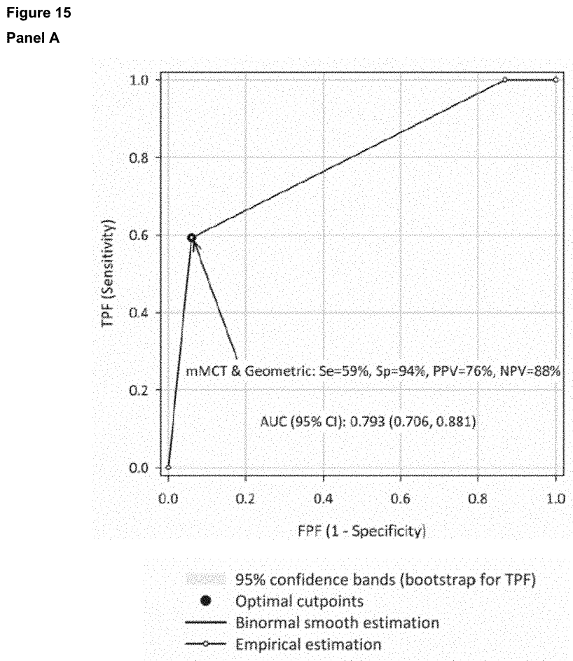

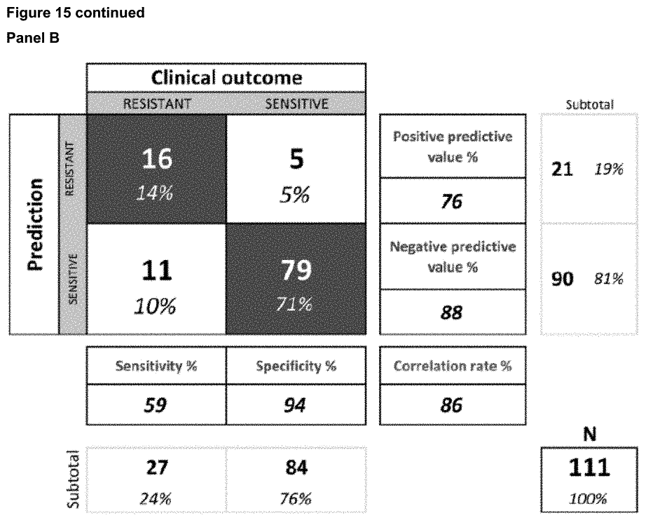

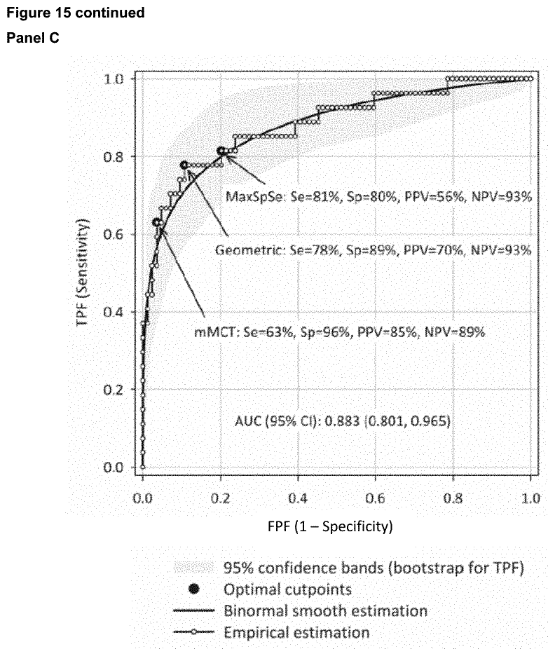

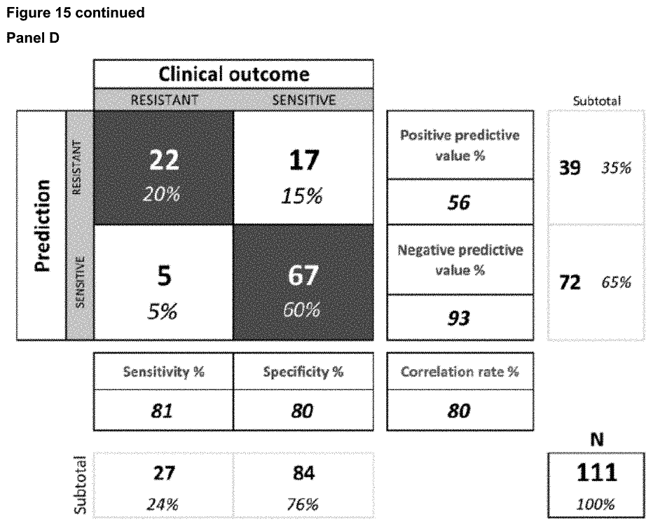

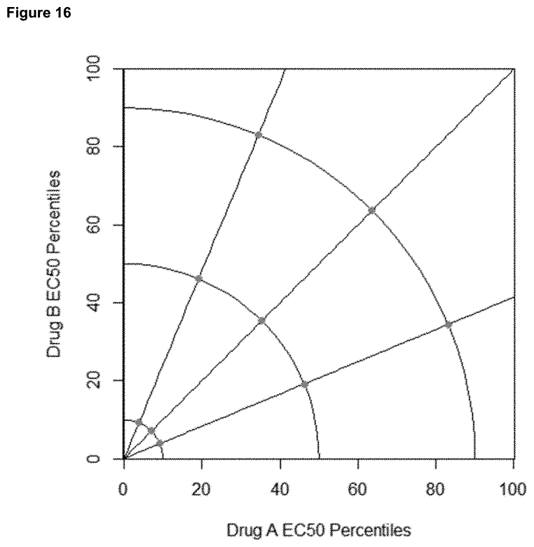

The OS was significantly shorter in patients predicted to be non-responders than in patients predicted to be responders regardless of the cut-point used to classify them (in other words, the estimated OS was significantly better in patients predicted to be responders). The median OS among patients predicted to be non-responders ranged from 344 to 589 days (FIG. 14). It was not reached in patients predicted to be responders. The hazard ratios (HR) of death (patients predicted to be non-responders vs. responders) ranged from 2.46 (1.38-4.36, panel A) to 3.44 (1.88-6.28, panel C). The values for the groups defined by actual clinical response were similar (median OS among non-responders: 279 days; HR [resistant vs. CR/CRi]: 3.17).

### CONCLUSIONS

This novel approach to ex vivo testing using the PharmaFlow PM platform provided drug sensitivity parameters that were integrated in a flexible generalized additive logistic regression model with an outstanding predictive accuracy for hematological response after front-line induction with IDA-CYT 3+7. After validation in an external cohort, our diagnostic tool could be useful to select AML patients for 3+7 regimen vs. alternative schedules.

### Example 2

The Precision Medicine (PM) Test as described ranks treatments by a score that estimates the activity of each treatment across a population of patient samples. Higher score represents more ex vivo sensitivity to treatment, while lower score represents less ex vivo sensitivity to treatment, i.e. more ex vivo resistance. A particular format of the results of the PM Test is shown in FIG. 19, panels A and B, wherein the ranking of treatments by activity score from 100%=best to 0%=worst is shown (score may be represented graphically using a colour/tone gradient or categorisation). The following 3 categories are identified:

1. Very sensitive to treatments, expected to be sensitive in the patient, with scores between 80-100% (top)

2. Intermediate sensitivity to treatments, without any inference in terms of patient sensitivity, 20-80% (middle)

3. Very resistant to treatments, with score between 0-20% (bottom)

The PM Test format in FIG. 19 compares 32 different treatments for each patient type (sensitive, standard, resistant and very resistant patient types), ranking them in lists by score with the most sensitive treatment on top of each list. In the case of the sensitive patient, there is only one treatment (cf. top-most treatment) to which said disease was very sensitive ex vivo, and thus is expected to be sensitive to in the patient. There are five ex vivo treatments at the bottom of the list for said sensitive patient against which the disease is predicted resistant.

To validate the predictive power of these PM Test score results, the set of 123 samples treated homogeneously with CYT+IDA that were used in the correlation study of Example 1 were employed. Only 112 samples were used for validation. To evaluate the predictive power of the PM Test score, in the format shown in FIG. 19, panel A referring to a sensitive patient we can only use patients predicted to be sensitive or resistant. Treatments having intermediate scores are not considered sufficiently strong to predict any response. In the case of intermediate scores, other factors such as pharmacokinetics may be more important than the score. On the other hand, extreme scores are more likely to prevail over other factors such as pharmacokinetics.

The set of data of 112 patients treated with CYT+IDA represents a 1st line treatment of AML with a 72% response rate. This means that 72% of these 112 patients achieved Complete Remission (CR) clinically. The majority of patients were sensitive, with fewer resistant patients. Thus, the statistics are improved by calculating the % of cases in which the PM Test score predicted patients to be sensitive to the ex vivo treatment. This means selecting only the treatments to which the patient is expected to be very sensitive, with scores between 80-100%. The % of accurate prediction of sensitive (responsive) patients is referred as the Negative Predicted Value (NPV).

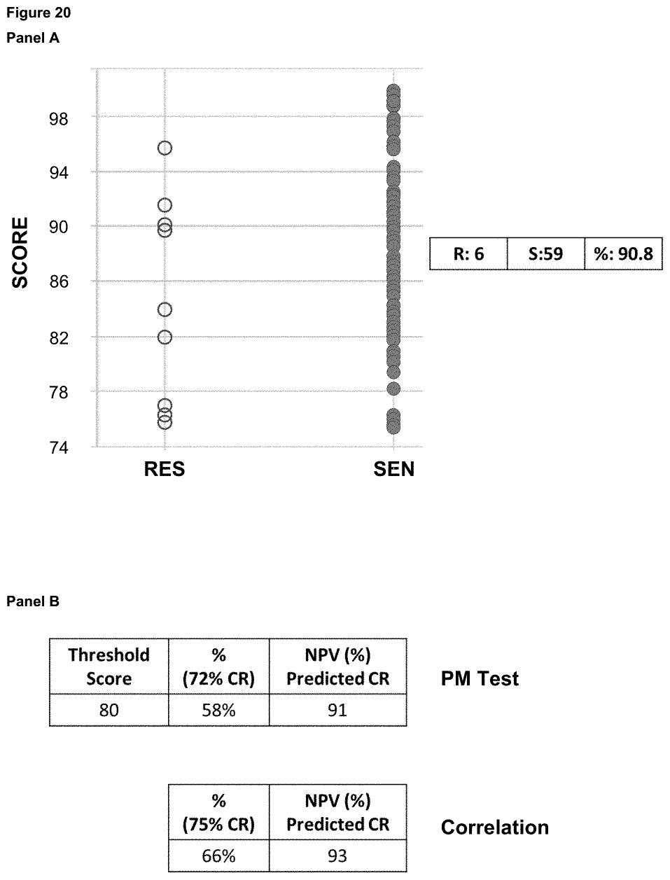

Table 4 and FIG. 20 show the results of this analysis. The table in the top panel shows the different thresholds chosen for the score, from 55 to 100. Samples whose score are between this variable threshold and 100% are analysed and predicted as sensitive because they have the top score. The results are shown for the monotherapies CYT and IDA, and for the combination treatment. Focus was placed on the combination treatment CYT+IDA that was administered to patients. Columns 8 to 10 of Table 4 show the results for the combined treatment CYT+IDA for different thresholds. The lowest threshold 55% (line 3), which means between 55 and 100%, identifies 93 out of the 112 samples, that would be predicted as sensitive. However, the results in columns 8 and 9 of Table 4 show that of these 93 patients clinically there were 16 which were resistant and 77 which were sensitive. Thus, the prediction accuracy or NPV is 77/93=82.8%. As the threshold is increased (descending the 1st column of Table 4), the range of scores included is narrowed because the maximum is always 100%. Consequently, as the threshold is increased, the total number of samples included diminishes, reaching 0 samples at 100% (100-100%) and 18 samples at 95%. The decrease can be observed in the number of either sensitive or resistant samples (descending columns 8 and 9 of Table 4). However, the NPV or predictive accuracy (% SEN, column 10 of Table 4) increases up to 94.4% for the 95-100% selection. Thus, the higher the score, the better the prediction (moreover, the intermediate sensitivity values provide less accurate prediction than the extreme values).

A threshold of 80% was selected for the PM Test because it provides a 91% NPV prediction accuracy: this is a similar prediction accuracy to the correlation analysis shown in Example 1 on the equivalent set of samples (123 instead of 112). The line for 80% threshold is highlighted in italics in Table 4. A total of 65 patients is included in this 80-100% threshold (6+59=65). This means 65 out of 112 patients have a score in the top 20%, i.e. the distribution of patient samples across the score is asymmetrical: a higher % of samples have higher scores than lower scores. The reason for this asymmetry is attributed to the high response rates in first-line AML treatment, with 72% of patients being sensitive and achieving complete response. Hence, the asymmetrical distribution of samples concentrating them in higher scores is consistent with the overall clinical response observed. FIG. 20, panel A shows the distribution of sensitive and resistant samples classified according to their score using this 80% threshold, as well as the final NPV or predictive accuracy of 91%.

TABLE 4
Numbers of resistant and numbers and percentage of sensitive patients
having a score greater than or equal to that indicated

| | CYT | | | IDA | | | CYT + IDA | | |
|---|---|---|---|---|---|---|---|---|---|
| Score | | | % | | | % | | | % |
| (≥) | Res. | Sens. | Sens. | Res. | Sens. | Sens. | Res. | Sens. | Sens. |
| 55 | 14 | 78 | 84.8 | 12 | 67 | 84.8 | 16 | 77 | 82.8 |
| 60 | 13 | 78 | 85.7 | 11 | 62 | 84.9 | 16 | 76 | 82.6 |
| 65 | 13 | 76 | 85.4 | 9 | 60 | 87.0 | 14 | 74 | 84.1 |
| 70 | 13 | 74 | 85.1 | 9 | 58 | 86.6 | 11 | 69 | 86.3 |
| 75 | 11 | 68 | 86.1 | 9 | 52 | 85.2 | 9 | 64 | 87.7 |
| 80 | 10 | 62 | 86.1 | 8 | 48 | 85.7 | 6 | 59 | 90.8 |
| 85 | 8 | 54 | 87.1 | 7 | 45 | 86.5 | 4 | 48 | 92.3 |
| 90 | 8 | 46 | 85.2 | 5 | 38 | 88.4 | 2 | 34 | 94.4 |
| 95 | 7 | 36 | 83.7 | 1 | 30 | 96.8 | 1 | 17 | 94.4 |
| 100 | 0 | 0 | | 0 | 0 | | 0 | 0 | |

Comparison of the prediction of the PM test score vs the correlation with clinical response data on this set of CYT+IDA shows that upon selecting a threshold of 80%, the PM test predicts 58% (65) of the 112 samples as sensitive with 91% accuracy (NPV). In contrast, the correlation predicts 66% of the samples as sensitive with 93% accuracy (NPV) (cf. FIG. 20, panel B). The predictive accuracy or NPV of both approaches is similar. The clinical response rate is also similar 72% for the PM test and 75% for the correlation. The key difference is that the correlation predicts 66% of samples to be sensitive, compared with the 75% who are sensitive clinically, while the PM test predicts 58% of samples to be sensitive, compared with the 72% who are sensitive clinically. Thus, the PM test is a much simpler approach than correlation analysis yet predicts sensitivity with similar accuracy.

### Example 3. Prediction of Clinical Response Vs Outcome after Induction Therapy Treatment with a Combination of Drugs Comprising a Drug A and a Drug B in Six Subjects Diagnosed with Acute Myeloid Leukaemia

Bone marrow samples were obtained from six adult patients diagnosed with AML (00281, 00183, 00218, 00096, 00086 and 00082). Following the same experimental design as described for Example 1, said bone marrow samples were incubated for 72 h in well plates containing the drugs pairs included in the combinations disclosed in Tables 5 and 6, or containing every individual drug in each combination. The same single drug and drug combination pharmacodynamic parameters as shown in Table 3 were determined. Pharmacological response parameters were estimated using population pharmacodynamic models. The strongest clinical predictors were the area under the curve (AUC) of the single drugs concentration response curves and the volume under the surface (VUS) in drugs concentrations interaction response surfaces. Score (S) for each combination was calculated using AUC and VUS values, once normalised, according to the expression of formula (XIX) defined herein and in claim 30.

Independently, patients 00281 and 00096 were treated with a drug combination comprising cytarabine (ARA-C) and idarubicin (IDA), patients 00183, 00086 and 00082 were treated with a drug combination comprising cytarabine (ARA-C) and daunorubicin (DNR), and patient 00218 was treated with a drug combination comprising cytarabine (ARA-C) and mitoxantrone (MIT). Said drug combinations were chosen for each patient prior to prediction of clinical response of any given aforementioned drug combination and the clinical outcome of treatment with said drug combination in each patient was determined as being resistant to or sensitive to said drug combination, whereby patients attaining a CR/CRi with up to two induction cycles were classified as sensitive to (i.e. responsive to) the drug combination and the remaining were classified as resistant.

Results: Score (S) According to the Ex Vivo Activity and Observed Clinical Response

The score for the particular drug combination with which each patient was treated was significantly lower in those patients predicted to be non-responders (00096, 00086 and 00082, cf. Table 6) than in patients predicted to be responders (00281, 00183 and 00218, cf. Table 5) regardless of the cut-off point used to classify them (in other words, the estimated score was significantly higher in patients predicted to be sensitive to treatment). Although the information from the PM test method of the present invention was not used for prescribing treatment, it can be seen that said PM test method would have correctly predicted the outcome with the drug combinations tested. Moreover, in the case of patient 00183, the treatment given to said patient coincided with the drug combination (cytarabine and daunorubicin) which also gave the highest score using the method of the present invention.

TABLE 5
Combinations of drugs to which acute myeloid leukaemia patients exhibit sensitivity
upon in vivo treatment, scores thereof, and scores of other combinations of drugs
Patient number

| | | |
|---|---|---|
| 00281 | 00183 | 00218 |

Clinical information (in vivo treatment)

| | | |
|---|---|---|
| ARA-C + IDA | ARA-C + DNR | ARA-C + MIT |

PM Test information

| Treatment in PM test | Score | Treatment in PM test | Score | Treatment in PM test | Score |
|---|---|---|---|---|---|
| ARA-C + Ida + Flu | 95.45 | ARA-C + DNR | 97.43 | ARA-C + FLU | 96.35 |
| ARA-C + IDA + CLA | 95.16 | ARA-C + IDA | 95.49 | ARA-C + MIT + FLU | 94.69 |
| ARA-C + IDA | 94.44 | ARA-C + DNR + FLU | 94.63 | ARA-C + IDA + FLU | 94.57 |
| ARA-C + MIT + FLU | 94.42 | ARA-C + IDA + FLU | 93.75 | ARA-C + MIT | 94.32 |
| ARA-C + MIT + CLA | 94.03 | ARA-C + DNR + ETO | 92.91 | ARA-C + IDA | 94.28 |
| ARA-C + DNR + FLU | 93.94 | ARA-C + IDA + ETO | 90.97 | ARA-C + IDA + ETO | 88.06 |
| ARA-C + DNR + CLA | 93.66 | ARA-C + FLU | 90.22 | ARA-C + MIT + ETO | 86.01 |
| ARA-C + DNR | 92.44 | ARA-C + MIT + FLU | 88.99 | FLU + MIT | 84.06 |
| ARA-C + MIT | 92.40 | ARA-C + ETO | 86.88 | Arac-C + DNR + FLU | 83.57 |
| ARA-C + FLU | 91.82 | ARA-C + MIT | 85.50 | ARA-C + ETO | 83.34 |
| ARA-C + CLA | 91.24 | ARA-C + MIT + ETO | 85.06 | FLU + IDA | 82.16 |
| ARA-C + IDA + ETO | 90.51 | FLU + DNR | 84.04 | MIT + ETO | 80.80 |
| FLU + MIT | 89.14 | FLU + IDA | 82.56 | ARA-C + DNR + ETO | 77.06 |
| FLU + IDA | 89.10 | IDA + ETO | 80.86 | ARA-C + DNR | 71.65 |
| ARA-C + DNR + ETO | 89.00 | FLU + MIT | 80.85 | IDA + ETO | 70.44 |
| CLA + MIT | 88.62 | DNR + ETO | 80.84 | FLU + DNR | 61.71 |
| CLA + IDA | 88.58 | MIT + ETO | 70.93 | DNR + ETO | 50.00 |
| ARA-C + MIT + ETO | 87.83 | | | | |
| FLU + DNR | 87.31 | | | | |
| CLA + DNR | 86.80 | | | | |
| ARA-C + ETO | 81.93 | | | | |
| DNR + ETO | 80.87 | | | | |
| MIT + ETO | 80.24 | | | | |
| IDA + ETO | 80.20 | | | | |

TABLE 6
Combinations of drugs to which acute myeloid leukaemia patients are resistant upon
in vivo treatment, scores thereof, and scores of other combinations of drugs
Patient number

| | | |
|---|---|---|
| 00096 | 00086 | 00082 |

Clinical information (in vivo treatment)

| | | |
|---|---|---|
| ARA-C + IDA | ARA-C + DNR | ARA-C + DNR |

PM Test information

| Treatment in PM test | Score | Treatment in PM test | Score | Treatment in PM test | Score |
|---|---|---|---|---|---|
| IDA + ETO | 19.61 | FLU + MIT | 70.55 | ARA-C + MIT | 19.93 |
| FLU + IDA | 19.53 | ARA-C + IDA + FLU | 69.79 | ARA-C + DNR + ETO | 19.73 |
| ARA-C + IDA | 19.52 | FLU + IDA | 67.90 | MIT + ETO | 19.34 |
| ARA-C + MIT | 19.49 | ARA-C + MIT + FLU | 67.83 | FLU + MIT | 19.33 |
| DNR + ETO | 19.48 | ARA-C + DNR + FLU | 61.87 | CLA + MIT | 19.32 |
| ARA-C + IDA + ETO | 19.48 | ARA-C + IDA | 57.88 | ARA-C + MIT + FLU | 19.25 |
| ARA-C + IDA + FLU | 19.47 | FLU + DNR | 57.26 | ARA-C + MIT + ETO | 19.25 |
| FLU + DNR | 19.39 | ARA-C + FLU | 50.11 | ARA-C + MIT + CLA | 19.24 |
| ARA-C + DNR | 19.38 | ARA-C + MIT | 50.03 | DNR + ETO | 19.21 |
| ARA-C + DNR + ETO | 19.38 | ARA-C + DNR | 43.58 | ARA-C + DNR + FLU | 17.47 |
| MIT + ETO | 19.37 | ARA-C + IDA + ETO | 19.56 | FLU + DNR | 17.20 |
| ARA-C + DNR + FLU | 19.35 | MIT + ETO | 19.56 | IDA + ETO | 17.09 |
| FLU + MIT | 19.31 | IDA + ETO | 19.53 | ARA-C + IDA + ETO | 16.48 |
| ARA-C + MIT + ETO | 19.27 | ARA-C + MIT + ETO | 19.51 | ARA-C + DNR | 16.10 |
| ARA-C + MIT + FLU | 19.23 | ARAC-C + DNR + ETO | 19.46 | FLU + IDA | 15.48 |
| ARA-C + ETO | 14.48 | ARA-C + ETO | 19.36 | ARA-C + IDA + FLU | 14.66 |
| ARA-C + FLU | 0.00 | DNR + ETO | 19.36 | ARA-C + DNR + CLA | 12.08 |
| | | | | ARA-C + IDA | 11.94 |
| | | | | ARA-C + ETO | 11.43 |
| | | | | ARA-C + IDA + CLA | 8.96 |
| | | | | CLA + DNR | 6.28 |
| | | | | CLA + IDA | 4.48 |
| | | | | ARA-C + FLU | 0.05 |
| | | | | ARA-C + CLA | 0.00 |

### Example 4. Prediction of Clinical Response Vs Outcome after Induction Therapy Treatment with a Combination of Drugs Comprising a Drug a and a Drug B, and Optionally a Drug C (or Further Drugs) in Three Subjects Diagnosed with Multiple Myeloma (MM) and Three Subjects Diagnosed with Acute Lymphoid Leukemia (ALL)

Bone marrow samples were obtained from three adult patients diagnosed with MM (VIVIA-PMMM010431, VIVIA-PMMM060111 and VIVIA-PMMM130061) and three adult patients diagnosed with ALL (VIVIA-PMALL07002, VIVIA-PMALL04001 and VIVIA-PMALL09001). Following the same experimental design as described for examples 1 and 3, said bone marrow samples were incubated for 48 h in well plates containing the single drugs shown in FIGS. 21 to 26. After the measurement of pathological populations by flow cytometry, pharmacological response parameters were estimated using population pharmacodynamic models. The most representative parameter of drug potency, the EC₅₀, was used for a comparative analysis of the results provided by said bone marrow samples with respect to the statistical distribution of the same parameter observed in a population of samples and stored in the database. Such populations varied in size depending on the drug, being always over one hundred individuals in the case of MM and between 15 and 21 individuals in the case of ALL.

FIG. 21 shows the comparative analysis of patient VIVIA-PMMM010431 who was treated with a combination of drugs comprising bortezomib, bendamustine and prednisolone and achieving complete response after induction treatment. Consistently, ex vivo results showed potent activity for the three drugs with EC₅₀ for bortezomib below the first quartile and below the 10th percentile for bendamustine and prednisolone.

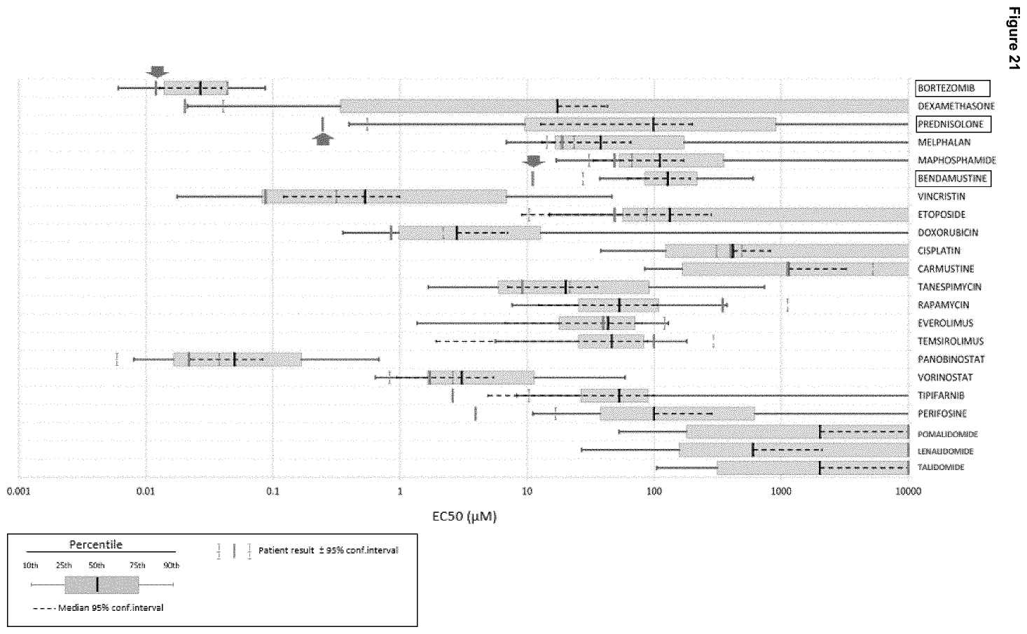

Patient VIVIA-PMMM060111 was treated with a combination of bortezomib and dexamethasone with a partial clinical response after induction. Results in the test showed (FIG. 22) an intermediate activity for bortezomib and no activity for dexamethasone. Consistently, all drugs from the same family according its mechanism of action as mTor inhibitors, provided similar and very sensitive results suggesting that any drugs from this family could be considered as an effective alternative treatment for this patient.

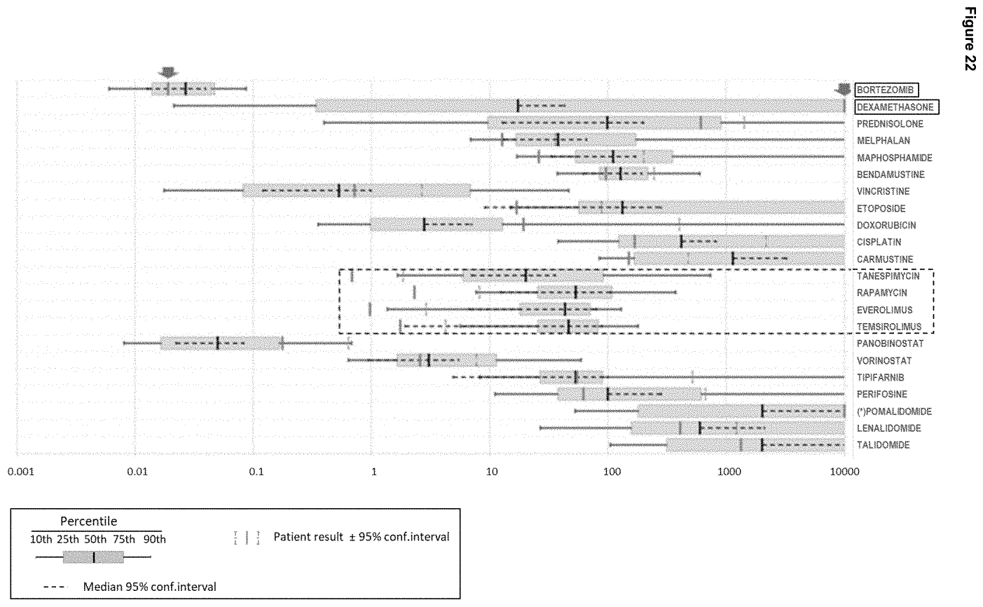

Patient VIVIA-PMMM130061 followed three different cycles of chemotherapy being resistant to all of them. The first cycle consisted of a combination of bortezomib and dexamethasone. The second cycle included the same drugs plus thalidomide and in the third cycle bortezomib and dexamethasone were combined with bendamustine instead. Ex vivo results of the tests showed very weak activity of the four drugs. Similarly, the test showed up to six different drugs with intermediate or high activity against tumor cells population which might have provided a better clinical response (FIG. 23).

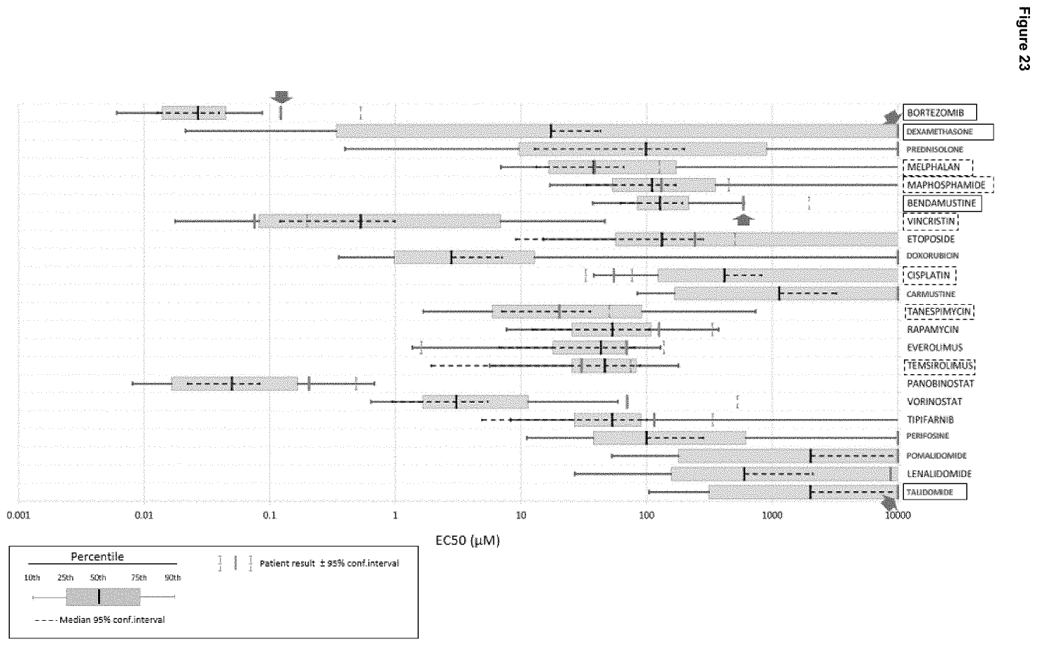

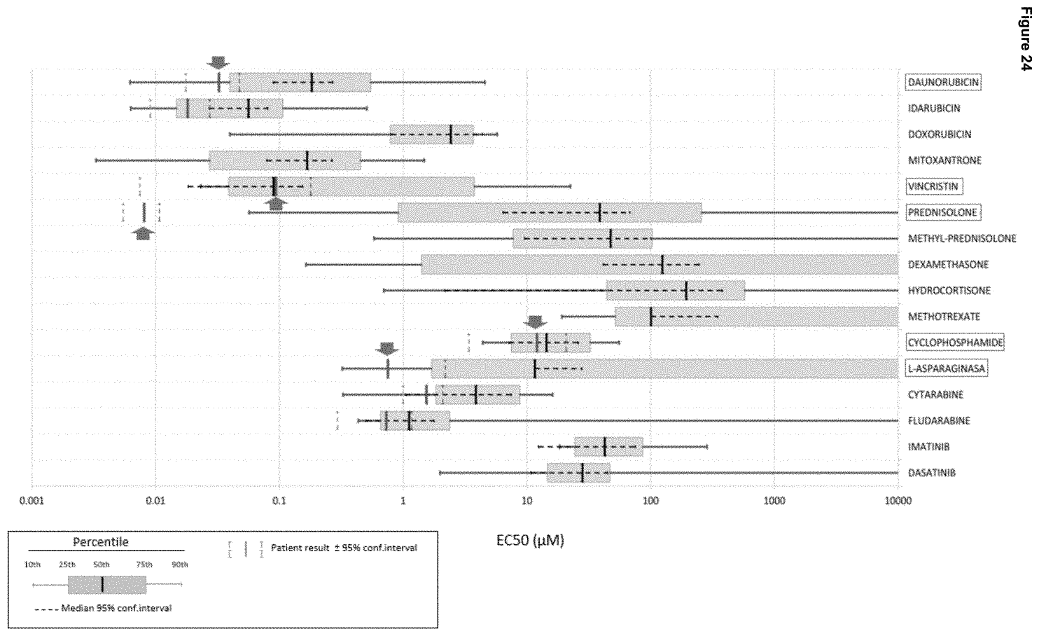

ALL patient VIVIA-PMALL07002 achieved a complete response after poly-therapy combining five different drugs: daunorubicin, vincristine, prednisolone, L-asparaginase and cyclophosphamide. Ex vivo test results showed intermediate or high activity of the five drugs as shown in FIG. 24.

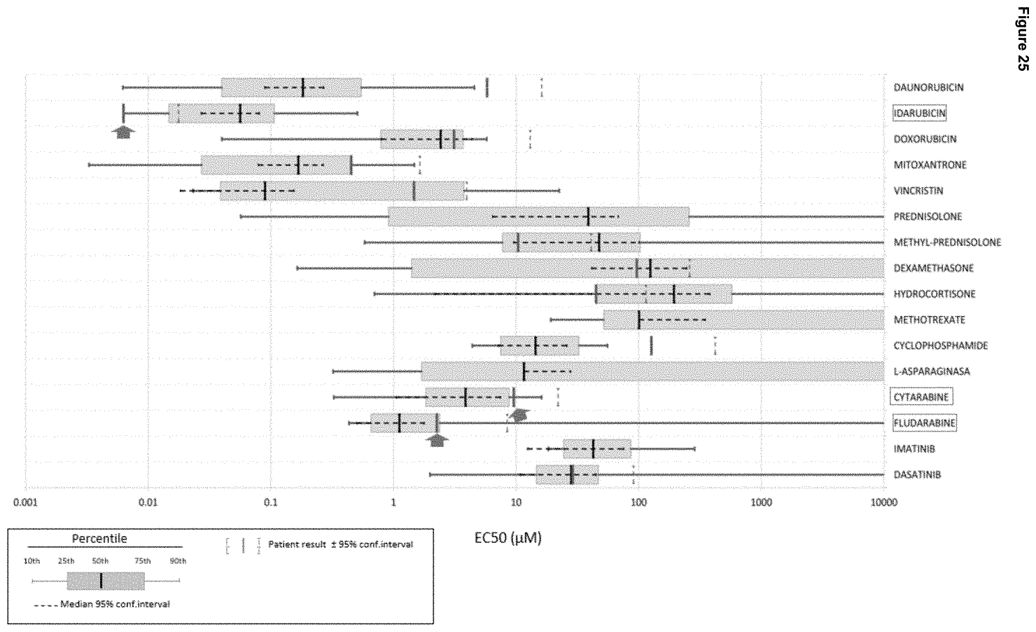

Second line patient VIVIA-PMALL04001 achieved complete response after a combination therapy using four drugs: fludarabine, idarubicin, cytarabine and prednisolone. In the ex vivo test idarubicin showed very potent activity whereas cytarabine and fludarabine showed intermediate-low activity. Drugs included in first-line treatment like daunorubicin, mitoxantrone or vincristine showed low activity or resistance in the ex vivo test (FIG. 25).

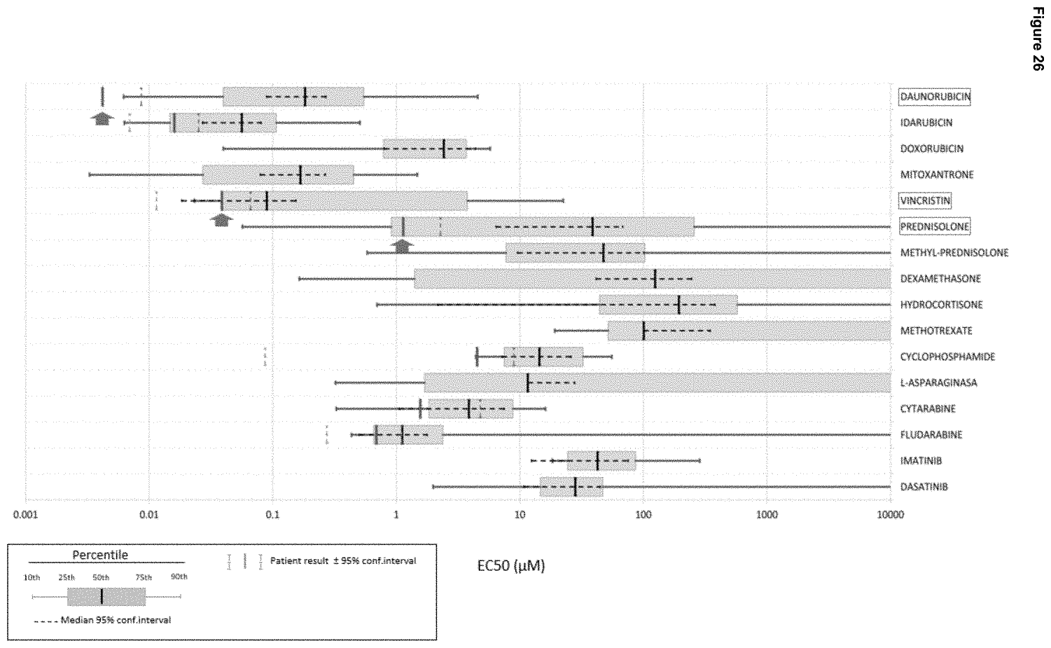

ALL patient VIVIA-PMALL09001 achieved a complete response after poly-therapy combining four different drugs: daunorubicin, vincristine, prednisolone and imatinib. Ex vivo test results showed high activity for all of these drugs except imatinib that could not be tested (FIG. 26).

### REFERENCES

1. Beal, S. L., Sheiner, L. B., Boeckmann, A. J. & Bauer, R. J. (1989-2001) NONMEM Users Guides. Icon Development Solutions, Ellicot City, Md.
2. Bennett, T. A., Montesinos, P., Moscardo, F., Martinez-Cuadron, D., Martinez, J., Sierra, J., Garcia, R., de Oteyza, J. P., Fernandez, P., Serrano, J., Fernandez, A., Herrera, P., Gonzalez, A., Bethancourt, C., Rodriguez-Macias, G., Alonso, A., Vera, J. A., Navas, B., Lavilla, E., Lopez, J. A., Jimenez, S., Simiele, A., Vidriales, B., Gonzalez, B. J., Burgaleta, C., Hernandez Rivas, J. A., Mascunano, R. C., Bautista, G., Perez Simon, J. A., Fuente Ade, L., Rayon, C., Troconiz, I. F., Janda, A., Bosanquet, A. G., Hernandez-Campo, P., Primo, D., Lopez, R., Liebana, B., Rojas, J. L., Gorrochategui, J., Sanz, M. A. & Ballesteros, J. (2014) Pharmacological profiles of acute myeloid leukemia treatments in patient samples by automated flow cytometry: a bridge to individualized medicine. Clin Lymphoma Myeloma Leuk, 14, 305-318.
3. Bergstrand, M., Hooker, A. C., Wallin, J. E. & Karlsson, M. O. (2011) Prediction-corrected visual predictive checks for diagnosing nonlinear mixed-effects models. AAPS J, 13, 143-151.
4. Greco, W. R., Bravo, G. & Parsons, J. C. (1995) The search for synergy: a critical review from a response surface perspective. Pharmacol Rev, 47, 331-385.
5. Greiner, M. (1996) Two-graph receiver operating characteristic (TG-ROC): update version supports optimisation of cut-off values that minimise overall misclassification costs. J Immunol Methods, 191, 93-94.
6. Grimwade, D., Hills, R. K., Moorman, A. V., Walker, H., Chatters, S., Goldstone, A. H., Wheatley, K., Harrison, C. J. & Burnett, A. K. (2010) Refinement of cytogenetic classification in acute myeloid leukemia: determination of prognostic significance of rare recurring chromosomal abnormalities among 5876 younger adult patients treated in the United Kingdom Medical Research Council trials. Blood, 116, 354-365.
8. Upton, R. N. & Mould, D. R. (2014) Basic concepts in population modeling, simulation, and model-based drug development: part 3-introduction to pharmacodynamic modeling methods. CPT Pharmacometrics Syst Pharmacol, 3, e88.
9. Vardiman, J. W., Harris, N. L. & Brunning, R. D. (2002) The World Health Organization (WHO) classification of the myeloid neoplasms. Blood, 100, 2292-2302.
10. Wood, S. N. (2006) Generalized Additive Models. An Introduction with R. Chapman & Hall/CRC, Boca Raton, Fla.

1.-27. (canceled)

28. A method for classifying the utility of combinations of drugs, each comprising a drug A and a drug B, in treatment of a subject diagnosed with a disease, wherein said method comprises the following steps:
(a) separating a tissue sample obtained from said subject into sub-samples;
(b) carrying out the steps of:
(i) incubating a sub-sample for a time T of between 2 and 168 hours in the presence of said drug A at a concentration X; and
(ii) repeating step (b)(i) an additional (N−1) times, each time with a different sub-sample using a value for X that is different from that used in previous repetitions of step (b)(i);
wherein N is a whole number selected from between 5 and 10, inclusive; and
(iii) incubating a sub-sample for said time T in the presence of said drug B at a concentration Y; and
(iv) repeating step (b)(iii) an additional (M−1) times, each time with a different sub-sample using a value for Y that is different from that used in previous repetitions of step (b)(iii);
wherein M is a whole number selected from between 5 and 10, inclusive; and
(v) incubating a sub-sample for said time T in the presence of a combination of drugs comprising said drug A and said drug B, wherein
the concentration of said drug A is a concentration X corresponding to the concentration at the percentile value P_{Hα,A} from the distribution of X_{50,A} values obtained in said population of subjects each diagnosed with said disease, wherein percentile value P_{Hα,A} is calculated by the formula (A):
P_{Hα,A}=cos(α°)×H (A)
wherein:
H corresponds to a reference percentile selected from the group of 10,
$$[ 10 + \frac{80}{R - 1} \times ( r - 1 ) ]$$
and 90, wherein:
r is a whole number selected from between 2 and (R−1), inclusive
α is in degrees and is calculated from the formula:
$$α = \frac{90}{( W + 1 )} \times w$$
wherein:
w is a whole number selected from between 1 and W, inclusive
the concentration of said drug B is a concentration Y corresponding to the concentration at the percentile value P_{Hα,B} from the distribution of Y_{50,B} values obtained in said population of subjects each diagnosed with said disease, wherein percentile value P_{Hα,B} is calculated by the formula (B):
P_{Hα,B}=cos(90°−α°)×H (B)
(vi) repeating step (b)(v) an additional (R−1) times, each time with a different sub-sample using a value for H that is different from that used in previous repetitions of step (b)(v), and using the same value for w that is used in step (b)(v); and
(vii) repeating steps (b)(v) and (b)(vi) an additional (W−1) times, each time with a different sub-sample using a value for w that is different from that used in previous repetitions of steps (b)(v) and (b)(vi);
wherein;
R is a whole number selected from between 3 and 10, inclusive;
W is a whole number selected from between 3 and 10, inclusive,
and wherein:
X_{50,A} is the concentration of drug A exerting half of the maximum activity in a subject, estimated according to step (e)(i), below;
Y_{50,B} is the concentration of drug B exerting half of the maximum activity in a subject, estimated according to step (e)(i), below;
and
(viii) incubating a sub-sample for said time T;
(c) adding at least one marker to each sub-sample incubated in step (b) to identify at least one cell-type (CTᵢ) therein;
(d) counting the number of live cells (LCTi) of each cell-type identified in step (c) which remain after incubation of each sub-sample according to step (b);
(e) determining for each cell-type identified in step (c):
(i) pharmacodynamic parameter values comprising at least one pharmacodynamic parameter value for drug A and/or at least one pharmacodynamic parameter value for drug B, wherein:
each pharmacodynamic parameter value for drug A is estimated from a single drug dose-response pharmacodynamic mixed effects non-linear population model by fitting a formula to experimental values of LCTi counted according to step (d) after incubating sub-samples for each subject in the population according to steps (b)(i) and (ii); and
each pharmacodynamic parameter value for drug B is estimated from a single drug dose-response pharmacodynamic mixed effects non-linear population model by fitting a formula to experimental values of LCTi counted according to step (d) after incubating sub-samples for each subject in the population according to steps (b)(iii) and (iv),
wherein said population comprises said subject and other subjects diagnosed with said disease;
(ii) activity marker values comprising at least one activity marker value for drug A, at least one activity marker value for drug B and/or at least one activity marker value for drugs A and B, wherein:
each activity marker value for drug A is calculated from said pharmacodynamic parameter value or values for drug A estimated in step (e)(i),
each activity marker value for drug B is calculated from said pharmacodynamic parameter value or values for drug B estimated in step (e)(i),
each activity marker value for drugs A and B is calculated from a specific model made by fitting a formula to said pharmacodynamic parameter value or values for drug A and said pharmacodynamic parameter value or values for drug B which are estimated in step (e)(i), as well as to experimental values of LCTi counted according to step (d) after incubating sub-samples for each subject in the population according to steps (b)(v) to (vii);
and
(iii) normalized marker values comprising at least one normalized marker value for drug A, at least one normalized marker value for drug B and/or at least one normalized marker value for drugs A and B, wherein:
each normalized marker value for drug A is calculated from the ratio of each activity marker value for drug A that is calculated in step (e)(ii) relative to a corresponding value from the distribution of said activity marker value for said population;
each normalized marker value for drug B is calculated from the ratio of each activity marker value for drug B that is calculated in step (e)(ii) relative to a corresponding value from the distribution of said activity marker value for drug B for said population;
each normalized marker value for drugs A and B is calculated from the ratio of each activity marker value for drugs A and B that is calculated in step (e)(ii) relative to a corresponding value from the distribution of said activity marker value for drugs A and B for said population;
(f) selecting:
(i) the pharmacodynamic parameter value or values determined according to step (e)(i) for each subject in said population of subjects; and/or
(ii) the activity marker value or values determined according to step (e)(ii) for each subject in said population of subjects; and/or
(iii) normalized marker value or values determined according to step (e)(iii) for each subject in said population of subjects, and/or
(iv) a clinical variable value or values for each subject in said population of subjects,
which are dependent on clinical resistance or clinical sensitivity to said combination of drugs, whereby a value is dependent when the probability of said value being independent from clinical resistance or clinical sensitivity is less than or equal to 0.05;
(g′) calculating a score S for treatment of said subject with said drug A and said drug B, wherein said score corresponds with or is calculated using at least one of the values which were selected in step (f);
(h′) carrying out steps (b) to (g′) for each combination of drugs to be classified;
and
(j′) classifying each combination of drugs using the score determined in steps (g′) and (h′), whereby a combination of drugs having a score of:
(i) greater than 80 is assigned to classification category I having a classification value of 2;
(ii) less than or equal to 80 and greater than 60 is assigned to classification category II having a classification value of 1;
(iii) less than or equal to 60 and greater than 40 is assigned to classification category III having a classification value of 0;
(iv) less than or equal to 40 and greater than 20 is assigned to classification category IV having a classification value of −1; or
(v) less than or equal to 20 is assigned to classification category V having a classification value of −2,
whereby:
each combination of drugs which is assigned to a classification category having a positive classification value or a classification value of zero is of highest utility in treatment of said disease in said subject; and
each combination of drugs which is assigned to classification category having a negative classification value is of lowest utility in treatment of said disease in said subject.

29. The method according to claim 28, wherein said combination is a combination of a drug A and a drug B and a drug C, wherein:
step (b) further comprises:
(ix) incubating a sub-sample for said time Tin the presence of said drug C at a concentration Z; and
(x) repeating step (b)(ix) an additional (L−1) times, each time with a different sub-sample using a value for Z that is different from that used in previous repetitions of step (b)(ix);
wherein L is a whole number selected from between 5 and 10, inclusive;
and
(xi) incubating a sub-sample for said time Tin the presence of a combination of drugs comprising said drug A and said drug C, wherein
the concentration of said drug A is a concentration X corresponding to the concentration at the percentile value P_{H′α′,A} from the distribution of X_{50,A} values obtained in said population of subjects each diagnosed with said disease, wherein percentile value P_{H′α′,A} is calculated by the formula (C):
P_{H′α′,A} cos(α°)×H′ (C)
wherein:
H′ corresponds to a reference percentile selected from the group of 10,
$$[ 10 + \frac{80}{{R}^{′} - 1} \times ( {r}^{′} - 1 ) ]$$
and 90, wherein:
r′ is a whole number selected from between 2 and (R′−1), inclusive
α′ is in degrees and is calculated from the formula:
$$α ’ = \frac{90}{( {W}^{′} + 1 )} \times {w}^{′}$$
wherein:
w′ is a whole number selected from between 1 and W′, inclusive
the concentration of said drug C is a concentration Z corresponding to the concentration at the percentile value P_{H′α′,C} from the distribution of Z_{50,C} values obtained in said population of subjects each diagnosed with said disease, wherein percentile value P_{H′α′,C} is calculated by the formula (D):
P_{H′α′,C}=cos(90°−α′°)×H′ (D)
(xii) repeating step (b)(xi) an additional (R′−1) times, each time with a different sub-sample using a value for H′ that is different from that used in previous repetitions of step (b)(xi), and using the same value for w′ that is used in step (b)(xi); and
(xiii) repeating steps (b)(xi) and (b)(xii) an additional (W′−1) times, each time with a different sub-sample using a value for w′ that is different from that used in previous repetitions of steps (b)(xi) and (b)(xii);
wherein;
R′ is a whole number selected from between 3 and 10, inclusive;
W′ is a whole number selected from between 3 and 10, inclusive,
and;
(xiv) incubating a sub-sample for said time T in the presence of a combination of drugs comprising said drug B and said drug C, wherein
the concentration of said drug B is a concentration Y corresponding to the concentration at the percentile value P_{H″α″,B} from the distribution of Y_{50,B} values obtained in said population of subjects each diagnosed with said disease, wherein percentile value P_{H″α′,B} is calculated by the formula (E):
P_{H″α′,B}=cos(α′″)×H″ (E)
wherein:
H″ corresponds to a reference percentile selected from the group of 10,
$$[ 10 + \frac{80}{{R}^{″} - 1} \times ( {r}^{″} - 1 ) ]$$
and 90, wherein:
R″ is a whole number selected from between 2 and (R″−1), inclusive
α″ is in degrees and is calculated from the formula:
$$α ’ = \frac{90}{( {W}^{″} + 1 )} \times {w}^{″}$$
wherein:
w″ is a whole number selected from between 1 and W″, inclusive
the concentration of said drug C is a concentration Z corresponding to the concentration at the percentile value P_{H″α″,C} from the distribution of Z_{50,C} values obtained in said population of subjects each diagnosed with said disease, wherein percentile value P_{H″α″,C} is calculated by the formula (F):
P_{H″α″,C}=cos(90°−α″°)×H″ (F)
(xv) repeating step (b)(xiv) an additional (R″−1) times, each time with a different sub-sample using a value for H″ that is different from that used in previous repetitions of step (b)(xiv), and using the same value for w″ that is used in step (b)(xiv); and
(xvi) repeating steps (b)(xiv) and (b)(xv) an additional (W″−1) times, each time with a different sub-sample using a value for w″ that is different from that used in previous repetitions of steps (b)(xiv) and (b)(xv);
wherein;
R″ is a whole number selected from between 3 and 10, inclusive;
W″ is a whole number selected from between 3 and 10, inclusive,
and wherein:
X_{50,A} is the concentration of drug A exerting half of the maximum activity in a subject, estimated according to step (e)(i);
Y_{50,B} is the concentration of drug B exerting half of the maximum activity in a subject, estimated according to step (e)(i);
Z_{50,C} is the concentration of drug C exerting half of the maximum activity in a subject, estimated according to step (e)(i), below;
the pharmacodynamic parameter values determined in step (e)(i) optionally additionally comprise at least one pharmacodynamic parameter value for drug C, wherein:
each pharmacodynamic parameter value for drug C is estimated from a single drug dose-response pharmacodynamic mixed effects non-linear population model by fitting a formula to experimental values of LCTi counted according to step (d) after incubating sub-samples for each subject in the population according to steps (b)(ix) and (x); and
the activity marker values determined in step (e)(ii) optionally additionally comprise at least one activity marker value for drug C, at least one activity marker value for drugs A and C, and/or at least one activity marker value for drugs B and C, wherein:
each activity marker value for drug C is calculated from said pharmacodynamic parameter value or values for drug C estimated in step (e)(i),
each activity marker value for drugs A and C is calculated from a specific model made by fitting a formula to said pharmacodynamic parameter value or values for drug A and said pharmacodynamic parameter value or values for drug C which are estimated in step (e)(i), as well as to experimental values of LCTi counted according to step (d) after incubating sub-samples for each subject in the population according to steps (b)(xi) to (xiii);
each activity marker value for drugs B and C is calculated from a specific model made by fitting a formula to said pharmacodynamic parameter value or values for drug B and said pharmacodynamic parameter value or values for drug C which are estimated in step (e)(i), as well as to experimental values of LCTi counted according to step (d) after incubating sub-samples for each subject in the population according to steps (b)(xiv) to (xvi);
the normalized marker values determined in step (e)(iii) optionally comprise normalized marker values comprising at least one normalized marker value for drug C, at least one normalized marker value for drugs A and C, and/or at least one normalized marker value for drugs B and C, wherein:
each normalized marker value for drug C is calculated from the ratio of each activity marker value for drug C that is calculated in step (e)(ii) relative to a corresponding value from the distribution of said activity marker value for said population;
each normalized marker value for drugs A and C is calculated from the ratio of each activity marker value for drugs A and C that is calculated in step (e)(ii) relative to a corresponding value from the distribution of said activity marker value for drugs A and C for said population;
each normalized marker value for drugs B and C is calculated from the ratio of each activity marker value for drugs B and C that is calculated in step (e)(ii) relative to a corresponding value from the distribution of said activity marker value for drugs B and C for said population.

30. The method according to claim 28, wherein
step (a) comprises separating a tissue sample obtained from said subject into at least 20 sub-samples;
the values for N and M in step (b) are identical and are a whole number selected from between 5 and 8, and the values for R and W in step (b) are identical and are a whole number selected from between 3 and 5;
the pharmacodynamic parameter values determined in step (e)(i) comprise an X_{50,A} value, a LCTi_{0,A} value, an E_{max,A} value, γ_{A} value, a Y_{50,B} value, an LCTi_{0,B} value, an E_{max,B} value and/or a γ_{B} value, wherein:
said X_{50,A}, LCTi_{0,A}, E_{max,A} and γ_{A} values are estimated from a single drug dose-response pharmacodynamic mixed effects non-linear population model determined by fitting the formula (I) to experimental values of LCTi counted according to step (d) after incubating sub-samples for each subject in a population according to steps (b)(i) and (b)(ii) obtained for each concentration X of drug A:
$$LCTi = {LCTi}_{0 , A} \times [ 1 - {E}_{max , A} \times \frac{{X}^{{γ}_{A}}}{{X}^{{γ}_{A}} + {X}_{50 , A}^{{γ}_{A}}} ] \qquad ( I )$$
said Y_{50,B}, LCTi_{0,B}, E_{max,B} and γ_{B} values are estimated from a single drug dose-response pharmacodynamic mixed effects non-linear population model determined by fitting the formula (II) to experimental values of LCTi counted according to step (d) after incubating sub-samples for each subject in said population according to steps (b)(iii) and (b)(iv) obtained for each concentration Y of drug B:
$$LCTi = {LCTi}_{0 , B} \times [ 1 - {E}_{max , B} \times \frac{{Y}^{{γ}_{B}}}{{Y}^{{γ}_{B}} + {Y}_{50 , B}^{{γ}_{B}}} ] \qquad ( II )$$
wherein said population comprises said subject and other subjects diagnosed with said disease;
wherein:
X=concentration of drug A;
X_{50,A} is the concentration of drug A exerting half of maximum activity;
LCTi_{50,A} is the basal (pre-incubation) number of LCTi and is equal to the LCTi counted after incubating a sub-sample in the absence of a drug according to the step referred to in (b)(viii);
E_{max,A}, is the maximum fractional decrease of LCTi_{0,A} caused by drug A;
γ_{A} is the steepness of the LCTi vs concentration curve for drug A;
Y=concentration of drug B;
Y_{50,B} is the concentration of drug B exerting half of maximum activity;
LCTi_{0,B} is the basal (pre-incubation) number of LCTi and is equal to the LCTi counted after incubating a sub-sample in the absence of a drug according to the step referred to in (b)(viii);
E_{max,B}, is the maximum fractional decrease of LCTi_{0,B} caused by drug B;
γ_{B} is the steepness of the LCTi vs concentration curve for drug B;
the activity marker values determined in step (e)(ii) comprise an AUC_{xy,A} value, an AUC_{xy,B} value, an α_{AB} value and/or a VUS_{AB} value, wherein:
said AUC_{xy,A} value is calculated using the formula (III):
AUC_{xy,A}=AUC_{x,A}−A_{y:10-90,A} (III)
wherein:
said AUC_{x,A} value is the integral between two drug concentrations X′ and X″ of a function derived from formula (I) for the % survival after incubating sub-samples according to steps (b)(i) and (b)(ii) obtained for each concentration X of drug A, wherein LCTi_{0,A} is considered as 100% survival, and is calculated using the formula (IV):
$$A U {C}_{x , A} = {\int }_{{X}^{′}}^{{X}^{″}} 100 \times [ 1 - {E}_{max , A} \times \frac{{X}^{{γ}_{A}}}{{X}^{{γ}_{A}} + {X}_{50 , A}^{{γ}_{A}}} ] d X \qquad ( IV )$$
wherein drug concentrations X′ and X″ correspond to the concentrations of the 20^{th} and 80^{th} percentiles of the X_{50,A} values obtained in said population of subjects each diagnosed with said disease, wherein X_{50,A} was calculated for each subject in said population according to steps (a) to (e)(i);
and
said A_{y:10-90,A} value is the surface from AUC_{x,A} that falls outside the 10% and 90% boundaries of the % survival, wherein LCTi_{0,A} is considered as 100% survival;
and
said AUC_{xy,B} value is calculated using the formula (V):
AUC_{xy,B}=AUC_{x,B}−A_{y:10-90,B} (V)
wherein:
said AUC_{x,B} value is the integral between two drug concentrations Y′ and Y″ of a function derived from formula (II) for the % survival after incubating sub-samples according to steps (b)(iii) and (b)(iv) obtained for each concentration Y of drug B, wherein LCTi_{0,B} is considered as 100% survival, and is calculated using the formula (VI):
$$A U {C}_{x , B} = {\int }_{{Y}^{′}}^{{Y}^{″}} 100 \times [ 1 - {E}_{max , B} \times \frac{{Y}^{{γ}_{B}}}{{Y}^{{γ}_{B}} + {Y}_{50 , B}^{{γ}_{B}}} ] d Y \qquad ( VI )$$
wherein drug concentrations Y′ and Y″ correspond to the concentrations of the 20^{th} and 80^{th} percentiles of the Y_{50,B} values obtained in said population of subjects each diagnosed with said disease, wherein Y_{50,B} was calculated for each subject in said population according to steps (a) to (e)(i);
and
said A_{y:10-90,B} value is the surface from AUC_{x,B} that falls outside the 10% and 90% boundaries of the % survival, wherein LCTi_{0,A} is considered as 100% survival;
and
said VUS_{AB} value is calculated using the formula (VII), wherein said VUS_{AB} value is the double integral between two drug concentrations X′ and X″ for drug A and two drug concentrations Y′ and Y″ for drug B of the model function of the natural log of LCTi counted after incubating sub-samples according to steps (b)(v) to (b)(vii), wherein LCTi_{0,A}=LCTi_{0,B} and is considered as 100% survival, and is calculated using the formula (VII),
$$( VII )$$
$$VU {S}_{A B} = {\int }_{{X}^{′}}^{{X}^{″}} {\int }_{{Y}^{′}}^{{Y}^{″}} 100 \times [ 1 - \frac{{( \begin{matrix}{E}_{max , A} \times \frac{X}{X + {X}_{50 , A}} + {E}_{max , B} \times \frac{Y}{Y + {Y}_{50 , B}} + \\ {α}_{A B} \times {E}_{max , A} \times {E}_{max , B} \times \frac{X}{X + {X}_{50 A}} \times \frac{Y}{Y + {Y}_{50 , B}}\end{matrix} )}^{{γ}_{int , AB}}}{( 1 + {( \frac{X}{{X}_{50 , A}} + \frac{Y}{{Y}_{50 , B}} + {α}_{A B} \times \frac{X}{{X}_{50 , A}} \times \frac{Y}{{Y}_{50 , B}} )}^{{γ}_{int , AB}} )} ]$$
wherein:
drug concentrations X′ and X″ correspond to the concentrations of the 20^{th} and 80^{th} percentiles of the X_{50,A} values obtained in said population of subjects each diagnosed with said disease, wherein X_{50,A} was calculated for each subject in said population according to steps (a) to (e)(i);
drug concentrations Y′ and Y″ correspond to the concentrations of the 20^{th} and 80^{th} percentiles of the Y_{50,B} values obtained in said population of subjects each diagnosed with said disease, wherein Y_{50,B} was calculated for each subject in said population according to steps (a) to (e)(i);
E_{max,A}=maximum fractional decrease in LPC caused by drug A;
E_{max,B}=maximum fractional decrease in LPC caused by drug B;
X_{50,A}=EC₅₀ concentration of drug A exerting half of E_{max,A};
Y_{50,B}=EC₅₀ concentration of drug B exerting half of E_{max,B};
X=concentration of drug A;
Y=concentration of drug B;
$${γ}_{A} \times \frac{\frac{X}{{X}_{50 , A}}}{\frac{X}{{X}_{50 , A}} + \frac{Y}{{Y}_{50 , B}}} + ( 1 - \frac{\frac{X}{{X}_{50 , A}}}{\frac{X}{{X}_{50 , A}} + \frac{Y}{{Y}_{50 , B}}} ) \times {γ}_{B}$$
$${γ}_{int , AB} = \text{?}$$
$$\text{?} \text{indicates text missing or illegible when filed}$$
wherein:
γA=steepness of the LCTi vs concentration curve for drug A;
γB=steepness of the LCTi vs concentration curve for drug B;
and
α_{AB}=synergy parameter estimated from a two-drug surface interaction model determined by fitting the formula (VII′) to experimental values of LCTi counted according to step (d) after incubating sub-samples for said subject according to the steps referred to in (b)(i) and (b)(ii) obtained for each concentration X of drug A, the steps referred to in (b)(iii) and (b)(iv) obtained for each concentration Y of drug B, and the steps referred to in b(v), b(vi) and b(vii) obtained for each pair of concentrations of the combination of drug A and drug B:
$$( {VII}^{′} )$$
$$LCTi = L C T {i}_{0 , AB} \times [ 1 - \frac{{( \begin{matrix}{E}_{max , A} \times \frac{X}{X + {X}_{50 , A}} + {E}_{max , B} \times \frac{Y}{Y + {Y}_{50 , B}} + {α}_{A B} \times \\ {E}_{max , A} \times {E}_{max , B} \times \frac{X}{X + {X}_{50 , A}} \times \frac{Y}{Y + {Y}_{50 , B}}\end{matrix} )}^{{γ}_{int , AB}}}{1 + {( \frac{X}{{X}_{50 , A}} + \frac{Y}{{Y}_{50 , B}} + {α}_{A B} \times \frac{X}{{X}_{50 , A}} \times \frac{Y}{{Y}_{50 , B}} )}^{{γ}_{int , AB}}} ]$$
the normalized marker values determined in step (e)(iii) comprise NAUC_{A}, NAUC_{B} and/or NVUS_{AB}, wherein:
NAUC_{A} is a normalised value for AUC_{xy,A} which is calculated using the formula (VIII);
NAUC_{A}=100×AUC_{xy,A}/AUC_{max,A} (VIII)
NAUC_{B} is a normalised value for AUC_{xy,B} which is calculated using the formula (IX);
NAUC_{B}=100×AUC_{xy,B}/AUC_{max,B} (IX)
NVUS_{AB} is a normalised value for VUS_{AB} which is calculated using the formula (X);
NVUS_{AB}=100×VUS_{AB}/VUS_{max,AB} (X)
wherein:
AUC_{max,A}=the maximum value for AUC_{xy,A} obtained in a population of subjects each diagnosed with said disease, wherein AUC_{xy,A} was calculated for each subject in said population according to steps (a) to (e)(ii);
AUC_{max,B}=the maximum value for AUC_{xy,B} obtained in said population of subjects each diagnosed with said disease, wherein AUC_{xy,B} was calculated for each subject in said population according to steps (a) to (e)(ii); and
VUS_{max,AB}=the maximum value for VUS_{AB} obtained in said population of subjects each diagnosed with said disease, wherein VUS_{AB} was calculated for each subject in said population according to steps (a) to (e)(ii);
score S is calculated in step (g′) using the formula (XIX):
$$s = ( \frac{\sum {( N A U C )}_{d} + \sum {( N V U S )}_{c d}}{D + C} ) \times f \qquad ( XIX )$$
wherein:
(NAUC)_{d}=normalised value NAUC for each drug included in said treatment;
(NVUS)_{cd}=normalised value NVUS for each combination of drugs included in said treatment;
D=number of drugs included in said treatment;
C=number of combinations of drugs included in said treatment; and
f=compensation factor for multiple drugs treatments, wherein:
$$f = ( 1 - ( \frac{( 3 - D )}{10} ) ) \qquad$$
or
score S is a value for NVUS selected from (NVUS)_{cd}.

31. The method according to claim 30, wherein said combination is a combination of a drug A and a drug B and a drug C, wherein:
step (a) comprises separating a tissue sample obtained from said subject into between 43 and 100 sub-samples;
the value for L in step (b) is identical with the values for N and M, and the values for R′, R″, W′ and W″ in step (b) are identical with the values for R and W;
the pharmacodynamic parameter values determined in step (e)(i) optionally further comprise a Z_{50,C} value, a LCTi_{0,C} value, an E_{max,C} value and/or a γ_{C} value, wherein:
said Z_{50,C}, LCTi_{0,C}, and γ_{C} values are estimated from a single drug dose-response pharmacodynamic mixed effects non-linear population model determined by fitting the formula (XI) to experimental values of LCTi counted according to step (d) after incubating sub-samples according to steps (b)(ix) and (b)(x) obtained for each concentration Z of drug C:
$$L C T i = {LCTi}_{0 , C} \times [ 1 - {E}_{max , C} \times \frac{{Z}^{{γ}_{C}}}{{Z}^{{γ}_{C}} + {Z}_{50 , C}^{{γ}_{C}}} ] \qquad ( XI )$$
wherein:
Z=concentration of drug C;
Z_{50,C} is the concentration of drug C exerting half of maximum activity;
LCTi_{0,C} is the basal (pre-incubation) number of LCTi and is equal to the LCTi counted after incubating a sub-sample in the absence of a drug according to the step referred to in (b)(viii);
E_{max,C} is the maximum fractional decrease of LCTi_{0,C} caused by drug C;
γ_{C} is the steepness of the LCTi vs concentration curve for drug C;
the activity marker values determined according to step (e)(ii) optionally further comprise an AUC_{xy,C} value, an α_{AC} value, a VUS_{AC} value, an α_{BC} value and/or a VUS_{BC} value, wherein:
said AUC_{xy,C} value is calculated using the formula (XII):
AUC_{xy,C}=AUC_{x,C}−A_{y:10-90,C} (XII)
wherein:
said AUC_{x,C} value is the integral between two drug concentrations Z′ and Z″ of a function derived from formula (XI) for the % survival after incubating sub-samples according to steps (b)(ix) and (b)(x) obtained for each concentration of drug C, wherein LCTi_{0,C} is considered as 100% survival, and is calculated using the formula (XIII):
$$A U {C}_{x , C} = {\int }_{{Z}^{′}}^{{Z}^{″}} 100 \times [ 1 - {E}_{max , C} \times \frac{{Z}^{{γ}_{C}}}{{Z}^{{γ}_{C}} + {Z}_{50 , C}^{{γ}_{C}}} ] d Z \qquad ( XIII )$$
wherein drug concentrations Z′ and Z″ correspond to the concentrations of the 20^{th} and 80^{th} percentiles of the Z_{50,C} values obtained in said population of subjects each diagnosed with said disease, wherein Z_{50,C} was calculated for each subject in said population according to steps (a) to (e)(i);
and
said A_{y:10-90,C} value is the surface from AUC_{x,C} that falls outside the 10% and 90% boundaries of the % survival, wherein LCTi_{0,C} is considered as 100% survival;
and
said VUS_{AC} value is calculated using the formula (XIV), wherein said VUS_{AC} value is the double integral between two drug concentrations X′ and X″ for drug A and two drug concentrations Z′ and Z″ for drug C of the model function of the natural log of LCTi counted after incubating sub-samples according to steps (b)(xi) to (b)(xiii), wherein LCTi_{0,A}=LCTi_{0,C} and is considered as 100% survival,
$$( XIV )$$
$$VU {S}_{A C} = {\int }_{{X}^{′}}^{{X}^{″}} {\int }_{{Z}^{′}}^{{Z}^{″}} 100 \times [ 1 - \frac{{( \begin{matrix}{E}_{max , A} \times \frac{X}{X + {X}_{50 , A}} + {E}_{max , C} \times \frac{Z}{Z + {Z}_{50 , C}} + {α}_{A C} \times \\ {E}_{max , A} \times {E}_{max , C} \times \frac{X}{X + {X}_{50 , A}} \times \frac{Z}{Z + {Z}_{50 , C}}\end{matrix} )}^{{γ}_{int , A C}}}{( 1 + {( \frac{X}{{X}_{50 , A}} + \frac{Z}{{Z}_{50 , C}} + {α}_{A C} \times \frac{X}{{X}_{50 , A}} \times \frac{Z}{{Z}_{50 , C}} )}^{{γ}_{int , A C}} )} ] dXdZ$$
wherein:
drug concentrations X′ and X″ correspond to the concentrations of the 20^{th} and 80^{th} percentiles of the X_{50,A} values obtained in said population of subjects each diagnosed with said disease, wherein X_{50,A} was calculated for each subject in said population according to steps (a) to (e)(i);
drug concentrations Z′ and Z″ correspond to the concentrations of the 20^{th} and 80^{th} percentiles of the Z_{50,C} values obtained in said population of subjects each diagnosed with said disease, wherein Z_{50,C} was calculated for each subject in said population according to steps (a) to (e)(i);
E_{max,A}=maximum fractional decrease in LPC caused by drug A;
E_{max,C}=maximum fractional decrease in LPC caused by drug C;
X_{50,A}=EC₅₀ concentration of drug A exerting half of E_{max,A};
Z_{50,C}=EC₅₀ concentration of drug C exerting half of E_{max,C};
X=concentration of drug A;
Z=concentration of drug C;
$${γ}_{int , A C} = {γ}_{A} \times \frac{\frac{X}{{X}_{50 , A}}}{\frac{X}{{X}_{50 , A}} + \frac{Z}{{Z}_{50 , C}}} + ( 1 - \frac{\frac{X}{{X}_{50 , A}}}{\frac{X}{{X}_{50 , A}} + \frac{Z}{{Z}_{50 , C}}} ) \times {γ}_{C}$$
wherein:
γA=steepness of the LCTi vs concentration curve for drug A;
γC=steepness of the LCTi vs concentration curve for drug C;
and
α_{AC}=synergy parameter estimated from a two-drug surface interaction model determined by fitting the formula (XIV′) to experimental values of LCTi counted according to step (d) after incubating sub-samples for said subject according to the steps referred to in (b)(i) and (b)(ii) obtained for each concentration X of drug A, the steps referred to in (b)(ix) and (b)(x) obtained for each concentration Z of drug C, and the steps referred to in b(xi), b(xii) and b(xiii) obtained for each pair of concentrations of the combination of drug A and drug C:
$$( {XIV}^{′} )$$
$$LCTi = L C T {i}_{0 , A C} \times [ 1 - \frac{{( \begin{matrix}{E}_{max , A} \times \frac{X}{X + {X}_{50 , A}} + {E}_{max , C} \times \frac{Z}{Z + {Z}_{50 , C}} + {α}_{A C} \times \\ {E}_{max , A} \times {E}_{max , C} \times \frac{X}{X + {X}_{50 , A}} \times \frac{Z}{Z + {Z}_{50 , C}}\end{matrix} )}^{{γ}_{int , A C}}}{1 + {( \frac{X}{{X}_{50 , A}} + \frac{Z}{{Z}_{50 , C}} + {α}_{A C} \times \frac{X}{{X}_{50 , A}} \times \frac{Z}{{Z}_{50 , C}} )}^{{γ}_{int , A C}}} ]$$
said VUS_{BC} value is calculated using the formula (XV), wherein said VUS_{BC} value is the double integral between two drug concentrations Y′ and Y″ for drug B and two drug concentrations Z′ and Z″ for drug C of the model function of the natural log of LCTi counted after incubating sub-samples according to steps (b)(xiv) to (b)(xvi), wherein LCTi_{0,B}=LCTi_{0,C} and is considered as 100% survival,
$$( XV )$$
$${VUS}_{BC} = {\int }_{{Y}^{′}}^{{Y}^{″}} {\int }_{{Z}^{′}}^{{Z}^{″}} 100 \times [ 1 - \frac{{( \begin{matrix}{E}_{max , B} \times \frac{Y}{{Y}_{50 , B}} + {E}_{max , C} \times \frac{Z}{Z + {Z}_{50 , C}} + {α}_{BC} \times \\ {E}_{max , B} \times {E}_{max , C} \times \frac{Y}{Y + {Y}_{50 , B}} \times \frac{Z}{Z + {Z}_{50 , C}}\end{matrix} )}^{{γ}_{int , BC}}}{1 + {( \frac{Y}{{Y}_{50 , B}} + \frac{Z}{{Z}_{50 , C}} + {α}_{BC} \times \frac{Y}{{Y}_{50 , B}} \times \frac{Z}{{Z}_{50 , C}} )}^{{γ}_{int , BC}}} ] dXdZ$$
wherein:
drug concentrations Y′ and Y″ correspond to the concentrations of the 20^{th} and 80^{th} percentiles of the Y_{50,B} values obtained in said population of subjects each diagnosed with said disease, wherein Y_{50,B} was calculated for each subject in said population according to steps (a) to (e)(i);
drug concentrations Z′ and Z″ correspond to the concentrations of the 20^{th} and 80^{th} percentiles of the Z_{50,C} values obtained in said population of subjects each diagnosed with said disease, wherein Z_{50,C} was calculated for each subject in said population according to steps (a) to (e)(i);
E_{max,B}=maximum fractional decrease in LPC caused by drug B;
E_{max,C}=maximum fractional decrease in LPC caused by drug C;
Y_{50,B}=EC₅₀ concentration of drug B exerting half of E_{max,B};
Z_{50,C}=EC₅₀ concentration of drug C exerting half of E_{max,C};
Y=concentration of drug B;
Z=concentration of drug C;
$${γ}_{int , BC} = {γ}_{B} \times \frac{\frac{Y}{{Y}_{50 , B}}}{\frac{Y}{{Y}_{50 , B}} + \frac{Z}{{Z}_{50 , C}}} + ( 1 - \frac{\frac{Y}{{Y}_{50 , B}}}{\frac{Y}{{Y}_{50 , B}} + \frac{Z}{{Z}_{50 , C}}} ) \times {γ}_{C}$$
wherein:
γB=steepness of the LCTi vs concentration curve for drug B;
γC=steepness of the LCTi vs concentration curve for drug C;
and
α_{BC}=synergy parameter estimated from a two-drug surface interaction model determined by fitting the formula (XV′) to experimental values of LCTi counted according to step (d) after incubating sub-samples for said subject according to the steps referred to in (b)(iii) and (b)(iv) obtained for each concentration Y of drug B, the steps referred to in (b)(ix) and (b)(x) obtained for each concentration Z of drug C, and the steps referred to in b(xiv), b(xv) and b(xvi) obtained for each pair of concentrations of the combination of drug B and drug C:
$$( {XV}^{′} )$$
$$LCTi = {LCTi}_{0 , BC} \times [ 1 - \frac{{( \begin{matrix}{E}_{max , B} \times \frac{Y}{Y + {Y}_{50 , B}} + {E}_{max , C} \times \frac{Z}{Z + {Z}_{50 , C}} + {α}_{BC} \times \\ {E}_{max , B} \times {E}_{max , C} \times \frac{Y}{Y + {Y}_{50 , B}} \times \frac{Z}{Z + {Z}_{50 , C}}\end{matrix} )}^{{γ}_{int , BC}}}{1 + {( \frac{Y}{{Y}_{50 , B}} + \frac{Z}{{Z}_{50 , C}} + {α}_{BC} \times \frac{Y}{{Y}_{50 , B}} \times \frac{Z}{{Z}_{50 , C}} )}^{{γ}_{int , BC}}} ]$$
the normalised marker values determined according to step (e)(iii) optionally further comprise NAUC_{C}, NVUS_{AC} and/or NVUS_{BC}, wherein:
NAUC_{C} is a normalised value for AUC_{xy,C} which is calculated using the formula (XVI);
NAUC_{C}=100×AUC_{xy,C}/AUC_{max,C} (XVI)
NVUS_{AC} is a normalised value for VUS_{AC} which is calculated using the formula (XVII);
NVUS_{BC}=100×VUS_{AC}/VUS_{max,AC} (XVII)
NVUS_{BC} is a normalised value for VUS_{BC} which is calculated using the formula (XVIII);
NVUS_{BC}=100×VUS_{BC}/VUS_{max,BC} (XVIII)
wherein:
AUC_{max,C}=the maximum value for AUC_{xy,C} obtained in said population of subjects each diagnosed with said disease, wherein AUC_{xy,C} was calculated for each subject in said population according to steps (a) to (e)(ii);
VUS_{max,AC}=the maximum value for VUS_{BC} obtained in said population of subjects each diagnosed with said disease, wherein VUS_{AC} was calculated for each subject in said population according to steps (a) to (e)(ii); and
VUS_{max,BC}=the maximum value for VUS_{BC} obtained in said population of subjects each diagnosed with said disease, wherein VUS_{BC} was calculated for each subject in said population according to steps (a) to (e)(ii).

32. The method according to claim 28, wherein when the number of subjects diagnosed with said disease for whom a score S has been calculated is equal to 20% of the population size used to calculate said score, said subjects are included in said population and the concentrations X, the concentrations Y, the drug concentrations X′ and X″, the drug concentrations Y′ and Y″ and, in the case of claim 4, the concentrations Z and the drug concentrations Z′ and Z″, are adjusted accordingly.

33. The method according to claim 28, wherein the disease is a cancer of the hematopoietic and lymphoid tissues.

34. The method according to claim 28, wherein said disease is acute myeloid leukaemia.

35. The method according to claim 28, wherein said subject is an adult subject and each subject in said population of subjects is an adult subject.

36. The method according to claim 28, wherein the bone-marrow cells are collected before the patient has been subjected to chemotherapy and/or radiation therapy.

37. The method according to claim 28, wherein:
the bone marrow cells have a viability greater than or equal to 60% when incubated for 48 hours in the absence of drug A and/or drug B and/or drug C; and/or
the bone-marrow cells were not present in a clot when obtained from said subject.

38. The method according to claim 28, which further comprises prescribing a plan of care to said subject, wherein said plan of care prescribes a combination of drugs selected from the combinations of drugs which are classified highest in utility for the treatment of said disease in said subject.

39.-50. (canceled)

51. A method of treatment of a subject diagnosed with a disease, comprising administration of a combination of drugs selected from the combinations of drugs which are classified highest in utility for the treatment of said disease in said subject according to the method of claim 28.

52.-54. (canceled)
